SEQUENCE LISTING (83 sequences)

SEQ ID NO: 1 - skipped

SEQ ID NO: 2 - RNA, 20 nt, Artificial Sequence
  5′ UTR Construct with “A” Kozak Sequence (no start codon)
gggagacauacucagccacc

SEQ ID NO: 3 - RNA, 20 nt, Artificial Sequence
  5′ UTR Construct with “G” Kozak Sequence (no start codon)
gggagacauacucagccgcc

SEQ ID NO: 4 - RNA, 29 nt, Artificial Sequence
  3′ UTR construct
  misc_feature (1)..(1): n is a, c, g, or u
  misc_feature (2)..(2): n can be present or absent
  misc_feature (3)..(3): n can be present or absent
  misc_feature (11)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnauaccucnnnnnnnnnnnnnnngagg

SEQ ID NO: 5 - RNA, 29 nt, Artificial Sequence
  3′UTR construct
  misc_feature (1)..(1): n is a, c, g, or u
  misc_feature (2)..(2): n can be present or absent
  misc_feature (3)..(3): n can be present or absent
  misc_feature (11)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnaaaccucnnnnnnnnnnnnnnngagg

SEQ ID NO: 6 - RNA, 29 nt, Artificial Sequence
  3′UTR construct
  misc_feature (1)..(1): n is a, c, g, or u
  misc_feature (2)..(2): n can be present or absent
  misc_feature (3)..(3): n can be present or absent
  misc_feature (11)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnauaggagnnnnnnnnnnnnnnncucc

SEQ ID NO: 7 - RNA, 29 nt, Artificial Sequence
  3′UTR construct
  misc_feature (1)..(1): n is a, c, g, or u
  misc_feature (2)..(2): n can be present or absent
  misc_feature (3)..(3): n can be present or absent
  misc_feature (11)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnaaaggagnnnnnnnnnnnnnnncucc

SEQ ID NO: 8 - RNA, 28 nt, Artificial Sequence
  3′UTR construct
  misc_feature (1)..(1): n is a, c, g, or u
  misc_feature (2)..(2): n can be present or absent
  misc_feature (3)..(3): n can be present or absent
  misc_feature (10)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnauagagnnnnnnnnnnnnnnnccuc

SEQ ID NO: 9 - RNA, 29 nt, Artificial Sequence
  3′UTR construct
  misc_feature (1)..(1): n is a, c, g, or u
  misc_feature (2)..(2): n can be present or absent
  misc_feature (3)..(3): n can be present or absent
  misc_feature (11)..(17): n is a, c, g, or u
  misc_feature (18)..(18): n can be present or absent
  misc_feature (19)..(19): n can be present or absent
  misc_feature (20)..(20): n can be present or absent
  misc_feature (21)..(21): n can be present or absent
  misc_feature (22)..(22): n can be present or absent
  misc_feature (23)..(23): n can be present or absent
  misc_feature (24)..(24): n can be present or absent
  misc_feature (25)..(25): n can be present or absent
nnnaaagaggnnnnnnnnnnnnnnnccuc

SEQ ID NO: 10 - RNA, 24 nt, Artificial Sequence
  3′UTR construct
ugcauaccucuaacggucuugagg

SEQ ID NO: 11 - RNA, 24 nt, Artificial Sequence
  3′UTR construct
ugcauaggaguaacggucuucucc

SEQ ID NO: 12 - RNA, 24 nt, Artificial Sequence
  3′UTR construct
ugcaaaggaguaacggucuucucc

SEQ ID NO: 13 - RNA, 27 nt, Artificial Sequence
  3′UTR construct
uaaugcauaccucuaacggucuugagg

SEQ ID NO: 14 - RNA, 27 nt, Artificial Sequence
  3′UTR construct
ugaugcauaccucuaacggucuugagg

SEQ ID NO: 15 - RNA, 27 nt, Artificial Sequence
  3′UTR construct
uagugcauaccucuaacggucuugagg

SEQ ID NO: 16 - RNA, 27 nt, Artificial Sequence
  3′UTR construct
uaaugcauagagguaacggucuuccuc

SEQ ID NO: 17 - RNA, 27 nt, Artificial Sequence
  3′UTR construct
ugaugcauagagguaacggucuuccuc

SEQ ID NO: 18 - RNA, 27 nt, Artificial Sequence
  3′UTR construct
uagugcauagagguaacggucuuccuc

SEQ ID NO: 19 - RNA, 27 nt, Artificial Sequence
  3′UTR construct
uaaugcaaagagguaacggucuuccuc

SEQ ID NO: 20 - RNA, 27 nt, Artificial Sequence
  3′UTR construct
ugaugcaaagagguaacggucuuccuc

SEQ ID NO: 21 - RNA, 27 nt, Artificial Sequence
  3′UTR construct
uagugcaaagagguaacggucuuccuc

SEQ ID NO: 22-23 - skipped (2 entries)

SEQ ID NO: 24 - DNA, 32 bp, Artificial Sequence
  Forward primer (No UTR)
gactcctaatacgactcactatagggagaatg

SEQ ID NO: 25 - DNA, 38 bp, Artificial Sequence
  Forward primer (Kozak)
gactcctaatacgactcactatagggagagccaccatg

SEQ ID NO: 26 - DNA, 46 bp, Artificial Sequence
  Forward primer (5′UTR)
gactcctaatacgactcactatagggagacatactcagccaccatg

SEQ ID NO: 27 - DNA, 64 bp, Artificial Sequence
  Reverse (3′UTR-A)
ttttttttttttttttttttttttttttttttttttttttcctcaagaccgttagaggtatgca

SEQ ID NO: 28 - DNA, 64 bp, Artificial Sequence
  Reverse (3′UTR-B)
ttttttttttttttttttttttttttttttttttttttttggagaagaccgttactcctatgca

SEQ ID NO: 29 - DNA, 64 bp, Artificial Sequence
  Reverse (3′UTR-C)
ttttttttttttttttttttttttttttttttttttttttggagaagaccgttactcctttgca

SEQ ID NO: 30 - DNA, 10 bp, Artificial Sequence
  Kozak sequence
gccrccatgg

SEQ ID NO: 31 - DNA, 23 bp, Artificial Sequence
  T7 promoter and hexamer
taatacgactcactatagggaga

SEQ ID NO: 32-33 - skipped (2 entries)

SEQ ID NO: 34 - RNA, 10 nt, Artificial Sequence
  Loop segment of stem loop
uaacggucuu

SEQ ID NO: 35-37 - skipped (3 entries)

SEQ ID NO: 38 - RNA, 23 nt, Artificial Sequence
  5′ UTR Construct with “A” Kozak Sequence (with start codon)
gggagacauacucagccaccaug

SEQ ID NO: 39 - RNA, 23 nt, Artificial Sequence
  5′ UTR Construct with “G” Kozak Sequence (with start codon)
gggagacauacucagccgccaug

SEQ ID NO: 40-44 - skipped (5 entries)

SEQ ID NO: 45 - DNA, 18 bp, Artificial Sequence
  DNA double-helix sequence
taatacgactcactatag

SEQ ID NO: 46-47 - skipped (2 entries)

SEQ ID NO: 48 - protein, 32 aa, Human immunodeficiency virus type 1
VGFPVTPQVPLRPMTYKAAVDLSHFLKEKGGL

SEQ ID NO: 49 - protein, 30 aa, Artificial Sequence
  DNA double helix
HTQGYFPDWQNYTPGPGVRYPLTFGWLYKL

SEQ ID NO: 50 - protein, 19 aa, Artificial Sequence
  DNA double helix
EKIRLRPGGKKKYKLKHIV

SEQ ID NO: 51 - protein, 32 aa, Artificial Sequence
  DNA double helix
NPPIPVGEIYKRWIILGLNKIVRMYSPTSILD

SEQ ID NO: 52 - protein, 31 aa, Artificial Sequence
  DNA double helix
AIFQSSMTKILEPFRKQNPDIVIYQYMDDLY

SEQ ID NO: 53 - protein, 20 aa, Plasmodium falciparum
NANPNANPNANPNANPNANP

SEQ ID NO: 54 - protein, 81 aa, Artificial Sequence
  E protein Domain III
AFTFTKIPAETLHTVTEVQYAGTDGPCKVPAQMAVDMQTLTPVGRLITANPVITEGTENSKMMLELDPPFGDSYIVIGVGE

SEQ ID NO: 55 - DNA, 827 bp, Artificial Sequence
  EGFP
gactcctaatacgactcactatagggagacatactcagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgcatacctctaacggtcttgaggaaaaaaaaaaaaaaaaaaaaaaaaaaaaaaaaaaaaaaaa

SEQ ID NO: 56 - RNA, 804 nt, Artificial Sequence
  EGFP
gggagacauacucagccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaaugcauaccucuaacggucuugaggaaaaaaaaaaaaaaaaaaaaaaaaaaaaaaaaaaaaaaaa

SEQ ID NO: 57 - DNA, 1190 bp, Homo sapiens
gactcctaatacgactcactatagggagacatactcagccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgatgcatacctctaacggtcttgaggaaaaaaaaaaaaaaaaaaaaaaaaaaaaaaaaaaaaaaaa

SEQ ID NO: 58 - RNA, 1167 nt, Homo sapiens
gggagacauacucagccaccauggcgggacaccuggcuucggauuucgccuucucgcccccuccaggugguggaggugaugggccaggggggccggagccgggcuggguugauccucggaccuggcuaagcuuccaaggcccuccuggagggccaggaaucgggccggggguugggccaggcucugaggugugggggauucccccaugccccccgccguaugaguucuguggggggauggcguacugugggccccagguuggaguggggcuagugccccaaggcggcuuggagaccucucagccugagggcgaagcaggagucgggguggagagcaacuccgauggggccuccccggagcccugcaccgucaccccuggugccgugaagcuggagaaggagaagcuggagcaaaacccggaggagucccaggacaucaaagcucugcagaaagaacucgagcaauuugccaagcuccugaagcagaagaggaucacccugggauauacacaggccgauguggggcucacccuggggguucuauuugggaagguauucagccaaacgaccaucugccgcuuugaggcucugcagcuuagcuucaagaacauguguaagcugcggcccuugcugcagaaguggguggaggaagcugacaacaaugaaaaucuucaggagauaugcaaagcagaaacccucgugcaggcccgaaagagaaagcgaaccaguaucgagaaccgagugagaggcaaccuggagaauuuguuccugcagugcccgaaacccacacugcagcagaucagccacaucgcccagcagcuugggcucgagaaggaugugguccgagugugguucuguaaccggcgccagaagggcaagcgaucaagcagcgacuaugcacaacgagaggauuuugaggcugcugggucuccuuucucagggggaccaguguccuuuccucuggccccagggccccauuuugguaccccaggcuaugggagcccucacuucacugcacuguacuccucggucccuuucccugagggggaagccuuucccccugucuccgucaccacucugggcucucccaugcauucaaacugaugcauaccucuaacggucuugaggaaaaaaaaaaaaaaaaaaaaaaaaaaaaaaaaaaaaaaaa

SEQ ID NO: 59 - DNA, 569 bp, Artificial Sequence
  IL2 Complete DNA Sequence (with Riboz UTRs and PolyA tail)
gactcctaatacgactcactatagggagacatactcagccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgatgcatacctctaacggtcttgaggaaaaaaaaaaaaaaaaaaaaaaaaaaaaaaaaaaaaaaaa

SEQ ID NO: 60 - RNA, 546 nt, Artificial Sequence
  Complete RNA Sequence (with Riboz UTRs and PolyA tail)
gggagacauacucagccaccauguacaggaugcaacuccugucuugcauugcacuaagucuugcacuugucacaaacagugcaccuacuucaaguucuacaaagaaaacacagcuacaacuggagcauuuacugcuggauuuacagaugauuuugaauggaauuaauaauuacaagaaucccaaacucaccaggaugcucacauuuaaguuuuacaugcccaagaaggccacagaacugaaacaucuucagugucuagaagaagaacucaaaccucuggaggaagugcuaaauuuagcucaaagcaaaaacuuucacuuaagacccagggacuuaaucagcaauaucaacguaauaguucuggaacuaaagggaucugaaacaacauucaugugugaauaugcugaugagacagcaaccauuguagaauuucugaacagauggauuaccuuuugucaaagcaucaucucaacacugacuugaugcauaccucuaacggucuugaggaaaaaaaaaaaaaaaaaaaaaaaaaaaaaaaaaaaaaaaa

SEQ ID NO: 61-62 - skipped (2 entries)

SEQ ID NO: 63 - RNA, 15 nt, Artificial Sequence
  5′ UTR construct
gggagagccaccaug

SEQ ID NO: 64 - RNA, 28 nt, Artificial Sequence
  Loop segment of stem loop
gaugccccauucacgaguaguggguauu

SEQ ID NO: 65 - RNA, 28 nt, Artificial Sequence
  Loop segment of stem loop
ggcacccugcgcaggugaugcaggugcc

SEQ ID NO: 66 - RNA, 28 nt, Artificial Sequence
  Loop segment of stem loop
guucgcucggucaggagagcugacggac

SEQ ID NO: 67 - RNA, 28 nt, Artificial Sequence
  Loop segment of stem loop
ucuuacaguggcaugugaccguuuaagg

SEQ ID NO: 68 - RNA, 28 nt, Artificial Sequence
  Loop segment of stem loop
cgcggcgcaugcacgugacaugccugcg

SEQ ID NO: 69 - RNA, 28 nt, Artificial Sequence
  Loop segment of stem loop
cggucccguggcaagagucuauggauug

SEQ ID NO: 70 - RNA, 28 nt, Artificial Sequence
  Loop segment of stem loop
auguucggcuccaagagcgaguugauau

SEQ ID NO: 71 - RNA, 28 nt, Artificial Sequence
  Loop segment of stem loop
cgauucgggcacaugugcugucugauug

SEQ ID NO: 72 - RNA, 28 nt, Artificial Sequence
  Loop segment of stem loop
guauucugaugcacgugccaucaaguac

SEQ ID NO: 73 - RNA, 28 nt, Artificial Sequence
  Loop segment of stem loop
uugagcaggaucaagugcauucuuucaa

SEQ ID NO: 74 - RNA, 28 nt, Artificial Sequence
  Loop segment of stem loop
rryryyyyryyyryrrryrryrrryryy

SEQ ID NO: 75 - RNA, 28 nt, Artificial Sequence
  Loop segment of stem loop
rryryyyyryryrrryrryryrrryryy

SEQ ID NO: 76 - RNA, 28 nt, Artificial Sequence
  Loop segment of stem loop
ryyyryyyrryyrrrrrrryyrryrrry

SEQ ID NO: 77 - RNA, 28 nt, Artificial Sequence
  Loop segment of stem loop
yyyyryrryrryryryrryyryyyrrrr

SEQ ID NO: 78 - RNA, 28 nt, Artificial Sequence
  Loop segment of stem loop
yryrryryryryryryrryryryyyryr

SEQ ID NO: 79 - RNA, 28 nt, Artificial Sequence
  Loop segment of stem loop
yrryyyyryrryrrrrryyyryrrryyr

SEQ ID NO: 80 - RNA, 28 nt, Artificial Sequence
  Loop segment of stem loop
ryryyyrryyyyrrrrryrrryyrryry

SEQ ID NO: 81 - RNA, 28 nt, Artificial Sequence
  Loop segment of stem loop
yrryyyrrryryryryryyryyyrryyr

SEQ ID NO: 82 - RNA, 28 nt, Artificial Sequence
  Loop segment of stem loop
ryryyyyrryryryryryyryyrrryry

SEQ ID NO: 83 - RNA, 28 nt, Artificial Sequence
  Loop segment of stem loop
yyrrryrrrryyrrryryryyyyyyyrr

(19) United States
(12) Patent Application Publication
(10) Pub. No.: US 2024/0175048 A1
(43) Pub. Date: May 30, 2024

(54) ENGINEERED EXPRESSION CONSTRUCTS TO INCREASE PROTEIN EXPRESSION FROM SYNTHETIC RIBONUCLEIC ACID (RNA)

(71) Applicant: Riboz, LLC, Spokane, WA (US)

(72) Inventor: Kambiz Mousavi, Seattle, WA (US)

(73) Assignee: Riboz, LLC, Spokane, WA (US)

(21) Appl. No.: 18/548,027

(22) PCT Filed: Feb. 25, 2022
(86) PCT No.: PCT/US2022/017880

§ 371 Date: Aug. 25, 2023

Related U.S. Application Data
(60) Provisional application No. 63/153,877, filed on Feb. 25, 2021.

(51) Int. Cl. C12N 15/85

(52) U.S. Cl. CPC C12N 15/85; C12N 2310/531; C12N 2830/50

(57) ABSTRACT

The present disclosure relates to enhancing protein expression in a cell by utilizing translational enhancing elements in the 5′ and/or 3′ untranslated region (UTR) of synthetic mRNA. The 5′ UTRs include a promoter, mini-enhancer sequence, and a Kozak sequence whereas the 3′ UTR includes a spacer, a stem loop structure, and optionally, a polyadenine tail. The artificial 5′ and 3′ UTRs increase protein expression, and in certain examples, do not include modified nucleosides, microRNA sites, or immune-evading factors.

### CROSS-REFERENCE TO RELATED APPLICATION

This application is a U.S. National Phase Patent Application based on International Patent Application No. PCT/US2022/017880, filed on Feb. 25, 2022, which claims priority to U.S. Provisional Patent Application No. 63/153,877 filed Feb. 25, 2021, the entire contents of both of which are incorporated by reference herein in their entirely.

### REFERENCE TO SEQUENCE LISTING

The Sequence Listing associated with this application is provided in text format in lieu of a paper copy and is hereby incorporated by reference into the specification. The name of the text file containing the Sequence Listing is 2YB1682 _ST25.txt. The text file is 27,759 bytes, was created on Aug. 24, 2023, and is being submitted electronically via Patent Center.

### FIELD OF THE DISCLOSURE

The current disclosure provides engineered expression constructs having artificial 5′ and/or 3′ untranslated regions (UTRs) flanking a coding sequence. The 5′ UTRs include a promoter, mini-enhancer sequence, and a Kozak sequence whereas the 3′ UTR includes a spacer, a stem loop structure, and optionally, a polyadenine tail. The artificial 5′ and 3′ UTRs increase protein expression, and in certain examples, do not include modified nucleosides, microRNA sites, or immune-evading factors.

### BACKGROUND OF THE DISCLOSURE

Prior existing methods to affect protein expression are riddled with problems. For example, it is possible for exogenously introduced deoxyribonucleic acid (DNA) to integrate into host cell genomic DNA with some frequency. This integration results in alterations and/or damage to the host cell genomic DNA. Alternatively, this exogenous DNA introduced into a cell can be inherited by daughter cells (whether or not the exogenous DNA has integrated into the chromosome) or by offspring. Further, even with proper delivery and no damage or integration into the host genome, multiple steps must occur before the encoded protein is produced from the DNA strand. Once inside the cell, DNA must be transported into the nucleus where it is transcribed into RNA. The RNA transcribed from DNA must then enter the cytoplasm where it is translated into protein. These multiple processing steps from transfected DNA to produced protein result in lag times before the eventual creation of a functional protein with each step representing an opportunity for error and damage to the cell. Further, often it is difficult to obtain the level of protein expression desired in cells because the transfected DNA may not express or not express at reasonable rates or concentrations necessary for the desired use. This can be a particular problem when DNA is introduced into primary cells or modified cell lines.

Messenger RNA (mRNA) has also been examined as a possibility to allow for short term modification of a cell. The advantages of using mRNA as a kind of reversible gene therapy include transient expression and a non-transforming character. mRNA does not need to enter the nucleus in order to be expressed and moreover cannot integrate into the host genome, thereby eliminating the risk of oncogenesis. Transfection rates attainable with mRNA are relatively high, for many cell types even >90%, and therefore, there is no need for selection of transfected cells.

Despite the significant recent developments to the mRNA-as-a-therapeutic and mRNA-as-a-vaccine fields, there remains a need in the art for further research into increasing the level of protein expression using mRNA.

### SUMMARY OF THE DISCLOSURE

### Technical Problem

An object of the present disclosure is to provide an engineered ribonucleic acid (e.g., mRNA) that increases the expression level of an encoded protein.

Another object of the present disclosure is to provide minimal sequences that increase the expression level of an encoded protein.

### Solutions to the Problems.

To this end, the current disclosure provides that certain engineered expression constructs (EEC) increase the expression level of an encoded protein. The EEC have artificial 5′ and/or 3′ untranslated regions (UTRs) flanking a coding sequence. The 5′ UTRs include a promoter, a mini-enhancer sequence (CAUACUCA, herein), and a Kozak sequence whereas the 3′ UTR includes a spacer, a stem loop structure, and optionally, a polyadenine tail (polyA tail). In certain examples, the 5′UTR is operably linked to a start codon to create an operational segment. In certain examples herein, the 3′ UTR is also depicted as including a stop codon.

Regarding the engineered 5′ UTR, in certain examples, the promoter is derived from a bacteriophage T7 promoter and has the sequence GGGAGA. In certain examples, the Kozak sequence includes GCCRCC wherein R is A or G. The Kozak sequence can also be operably linked to a start codon to create the sequence GCCRCC-start (e.g., GCCRCCAUG).

Particular embodiments of the 5′ UTR include from 5′ to 3′: a mini-T7 promoter, a mini-enhancer sequence (CAUACUCA, herein), and a Kozak sequence, thus creating GGGAGACAUACUCAGCCACC (SEQ ID NO: 2) or GGGAGACAUACUCAGCCGCC (SEQ ID NO: 3). The addition of start codons provides GGGAGACAUACUCAGCCACCAUG (SEQ ID NO: 38) and GGGAGACAUACUCAGCCGCCAUG (SEQ ID NO: 39).

In certain examples, these minimal 5′ UTR have less than 30 nucleotides. In certain examples, these minimal 5′ UTR have 20 or 23 nucleotides.

Regarding the engineered 3′ UTR, in certain examples, the spacer includes [N₁₋₃]AUA or [N₁₋₃]AAA. In more particular examples, the spacer includes UGCAUA or UGCAAA. Exemplary stem loop structures are formed by hybridizing sequences such as CCUC and GAGG. In certain examples, the loop structure formed between the hybridizing sequences is 7 to 15 nucleotides in length. An exemplary sequence of a loop segment includes UAACGGUCUU (SEQ ID NO: 34). When included as part of a 3′ UTR sequence, exemplary stop codons include UAA, UAG, and UGA.

In certain examples, these minimal 3′ UTR have less than 30 nucleotides.

Regarding the stem loop, increased protein expression is observed with EEC disclosed herein regardless of the order of the stem loop sequence, indicating that the secondary structure may be important, not necessarily the sequence in 5′ to 3′ orientation. The engineered 3′UTR can additionally include a polyA tail.

In one aspect, the present disclosure provides EEC including an in vitro-synthesized RNA including a coding sequence that encodes a protein of interest for translation in a mammalian cell, wherein said in vitro-synthesized RNA includes a 5′ UTR including the sequence CAUACUCA.

In one aspect, the present disclosure provides EEC including an in vitro-synthesized RNA including a coding sequence that encodes a protein of interest for translation in a mammalian cell, wherein said in vitro-synthesized RNA includes a 5′ UTR including SEQ ID NO: 2 or SEQ ID NO: 3. The 5′ UTR can also be presented with start codons to provide SEQ ID NO: 38 and SEQ ID NO: 39.

In one aspect, the present disclosure provides EEC including a 3′ UTR including SEQ ID NOs: 4, 5, 6, 7, 8, or 9.

In one aspect, the present disclosure provides EEC including a 3′ UTR including SEQ ID NOs: 10, 11, or 12.

In one aspect, the present disclosure provides EEC including a 3′ UTR including SEQ ID NOs: 13, 14, 15, 16, 17, 18, 19, 20, or 21.

Each of SEQ ID NOs: 4, 5, 6, 7, 8, 9, 10, 11, 12, 13, 14, 15, 16, 17, 18, 19, 20, and 21 may be constructed to include a polyA tail.

In one aspect, the present disclosure provides EEC including an in vitro-synthesized RNA including a coding sequence that encodes a protein of interest for translation in a mammalian cell, wherein said in vitro-synthesized RNA includes a 5′ UTR including the sequence CAUACUCA and a 3′ UTR including SEQ ID NOs: 4, 5, 6, 7, 8, 9, 10, 11, 12, 13, 14, 15, 16, 17, 18, 19, 20, and/or 21.

In one aspect, the present disclosure provides EEC including an in vitro-synthesized RNA including a coding sequence that encodes a protein of interest for translation in a mammalian cell, wherein said in vitro-synthesized RNA includes a 5′ UTR including SEQ ID NO: 2 or SEQ ID NO: 3 and a 3′ UTR including SEQ ID NOs: 4, 5, 6, 7, 8, 9, 10, 11, 12, 13, 14, 15, 16, 17, 18, 19, 20, and/or 21.

In one aspect, the present disclosure provides EEC including an in vitro-synthesized RNA including a coding sequence that encodes a protein of interest for translation in a mammalian cell, wherein said in vitro-synthesized RNA includes a 5′ UTR including SEQ ID NO: 38 or SEQ ID NO: 39 and a 3′ UTR including SEQ ID NOs: 4, 5, 6, 7, 8, 9, 10, 11, 12, 13, 14, 15, 16, 17, 18, 19, 20, and/or 21.

In particular embodiments, EEC disclosed herein do not include any modified nucleosides. In particular embodiments, EEC disclosed herein do not include any microRNA binding sites. In particular embodiments, EEC disclosed herein do not include any modified nucleosides and do not include any microRNA binding sites.

Within disclosed EEC, the engineered 5′ and 3′ UTRs flank a coding sequence within an open reading frame. Data provided in the current disclosure shows increased expression of green fluorescent protein (GFP), interleukin-2 (IL-2), and POU5F1 (OCT3/4) in a variety of cell types (e.g., lymphoid and adherent and suspension embryonic kidney cells), irrespective of the manner of transfection.

EEC disclosed herein can be utilized to increase expression of a variety of proteins for a number of different purposes. Exemplary purposes include in the use of therapeutics and vaccines.

### BRIEF DESCRIPTION OF THE FIGURES

Some of the figures may be better understood in color. Applicant considers the color version of the figures as part of the original submission and reserves the right to present color versions in later proceedings.

FIG. 1 shows a schematic of an EEC designed to increase protein expression in vivo. The EEC contains several modules within it to increase protein expression. Modules located within the 5′UTR are divided into three modules: module 1 (“M1”), which represents a promoter (e.g., a T7 promoter hexamer); module 2 (“M2”) which represents a unique translational enhancer (CAUACUCA, described herein); and module 3 (“M3”), which is the Kozak consensus sequence. The depicted exemplary 3′UTR is also divided into three segments including a stop codon, a spacer, and a stem-loop segment. A polyA tail may also be included.

FIG. 2 shows a flow cytometry histogram displaying GFP intensity (FL1-H) on the x-axis and cell counts on the y-axis. EXPI293 cells were transfected with increasing amount of EEC harboring the inventive 5′UTR (SEQ ID NO: 2) and 3′UTR (SEQ ID NO: 10) sequences, as described herein, and having a GFP coding sequence within its open reading frame (see SEQ ID NOs: 55 and 56). As shown, the GFP signal saturation is reached at 0.4 pmole (100 ng) of EEC 24 hours after transfection.

FIGS. 3A and 3B show flow cytometry graphs displaying GFP intensity (FL1-H) on the x-axis and cell counts on the y-axis for GFP-encoding EEC 5′UTR variants at three-hour post transfection (3A) and at 24 hours post transfection (3B). The results from the flow cytometry experiments are also provided as bar graphs. EXPI293 cells were transfected with equimolar amount of GFP-encoding EEC 5′UTR variants, no RNA (negative control) no 5′UTR mRNA, M1 and M3 modules only 5′UTR, and M1, M2 and M3 modules 5′UTR (SEQ ID NO: 2).

FIGS. 4A and 4B show flow cytometry graphs displaying GFP intensity (FL1-H) on the x-axis and cell counts on the y-axis for GFP-encoding EEC 5′UTR and 3′UTR variants at three-hour post transfection (4A) and at 24 hours post transfection (4B). The results from the flow cytometry experiment are also provided as bar graphs. EXPI293 cells transfected with equimolar amount of GFP-encoding EEC 5′UTR and 3′UTR variants:
(i) no RNA (negative control);
(ii) 3′UTR only mRNA (UGCAUACCUCUAACGGUCUUGAGG (SEQ ID NO: 10) or UAAUGCAUACCUCUAACGGUCUUGAGG (SEQ ID NO: 13) for the 3′UTR without a 5′UTR except the basic form of the UTR to allow transcription, GGGAGAAUG(ORF));
(iii) M3 only 5′UTR plus 3′UTR (UAAUGCAUACCUCUAACGGUCUUGAGG (SEQ ID NO: 13) as the 5′UTR and GGGAGAGCCACCAUG(ORF) (SEQ ID NO: 63) as the 3′UTR); and
(iv) M1, M2 and M3 5′UTR (SEQ ID NO: 2) plus 3′UTR (SEQ ID NO: 10). Note cells receiving EEC with full-length 5′ (SEQ ID NO: 2)+3′ (SEQ ID NO: 10) UTRs exhibit highest GFP intensities.

FIG. 5 illustrates the three different types of proteins that were expressed using EEC disclosed herein: targeted expression of proteins in the cytoplasm, organelle (i.e. nuclear compartment) and extracellular compartment (i.e. secretory proteins).

FIGS. 6A-6C diagrams HEK293 cells expressing GFP protein. (6A) depicts flow cytometry graphs displaying GFP intensity (x-axis) and the shift in detection that occurs with increasing amount of GFP-encoding EEC transfected into HEK293 cells (there is no GFP-encoding EEC mRNA in the panel labeled “No mRNA”; other panels show 0.2 pmoles of the GFP-encoding EEC mRNA; 0.4 pmoles of the GFP-encoding EEC mRNA, 1.0 pmoles of the GFP-encoding EEC mRNA, 2.0 pmoles of the GFP-encoding EEC mRNA and in 4.0 pmoles of the GFP-encoding EEC mRNA as indicated by the title of the panel). (6B) shows a chart interpreting the results from the flow cytometry graphs in 6A regarding the increase in percentage of GFP positive cells as relative to increasing administration of GFP-encoding EEC. To prepare the chart in 6B, the percentage of HEK293 cells positive for GFP is shown on the y-axis and the amount of GFP-encoding EEC mRNA is shown on the x-axis. As such, 6B depicts the increased percentage of GFP positive cells as the GFP-encoding EEC mRNA is increased. (6C) interprets the results from the flow cytometry graphs in 6A regarding the proportional increase in the median GFP intensity with increasing levels of transfected GFP-encoding EEC in cells that are positive for GFP expression. To prepare the chart in 6C, the median GFP intensity from cells expressing GFP is shown on the y-axis (FLI-H) and the amount of GFP-encoding EEC mRNA is shown on the x-axis. As such, 6C illustrates the proportional increase in the median GFP intensity as the GFP-encoding EEC mRNA is increased.

FIGS. 7A-7C show HEK293 cells expressing GFP protein after transfection with GFP-encoding EEC variants. (7A) illustrates a histogram that overlays GFP intensity (x-axis) of cells transfected with 0.4 pmole of GFP-encoding EEC variants. (7B) is similar to (7A) except cells are transfected with 1 pmole of GFP-encoding EEC variants. (7C) shows a bar graph displaying the median GFP intensity of HEK293 cells transfected with equimolar levels GFP-encoding EEC variants (n=2). The variants include 5′ UTR only (SEQ ID NO: 2); 3′ UTR only (as described in more detail in the description of FIG. 4B); 5′ and 3′ UTR (SEQ ID NO: 2 and SEQ ID NO: 10, as described in more detail in the description of FIG. 4B); Kozak only; and Kozak and 3′ UTR (SEQ ID NO: 10).

FIGS. 8A and 8B show Jurkat (T) lymphocytes expressing GFP protein after transfection with GFP-encoding EEC variants. (8A) displays flow cytometry graphs indicating the amount of GFP positive Jurkat cells existing in each cell group transfected with the various GFP-encoding EEC (full-length 5′ (SEQ ID NO: 2) and 3′ (SEQ ID NO: 10) UTRs. (8B) shows flow cytometry histograms indicating the amount of GFP positive Jurkat cells in each cell group transfected with the various GFP-encoding EEC 5′ (SEQ ID NO: 2) and 3′ (SEQ ID NO: 10) UTR EEC constructs. Results from the flow cytometry graphs in (8A) are provided as a line graph displaying the percentage of GFP positive Jurkat cells transfected with increasing amount of GFP-encoding EEC with full-length 5′ (SEQ ID NO: 2) and 3′ (SEQ ID NO: 10) UTRs. Results from the flow cytometry experiment of (8B) are provided as a bar graph displaying GFP intensity and percentage of Jurkat cells transfected with 8 pmoles of GFP-encoding EEC variants (n=2). Data are presented for variants no UTR; Kozak only; 5′ only; 3′ only; 5′ and 3′; and Kozak and 3′. Details about the sequences used in these variants can be found in the descriptions of FIG. 4B and FIG. 7C.

FIGS. 9A-9C shows the amount of GFP expression in Jurkat (T) lymphocytes 24 hours after electroporating the lymphocytes with GFP-encoding EEC harboring the UTR variants. (9A) displays representative examples of histograms with GFP intensity and percentage of Jurkat cells transfected using 4 pmoles of each GFP-encoding EEC variant. (9B) shows representative examples of histograms displaying GFP intensity and percentage of Jurkat cells transfected with 8 pmoles of each GFP-encoding EEC variant. (9C) is a bar graph displaying the median GFP intensity {number of experiments=2} of lymphocytes with 4 and 8 pmoles of GFP-encoding EEC variants, as shown in (9A) and (9B).

FIGS. 10A-10C show a representative experiment where Raji lymphocytes expressing GFP protein are subjected to flow cytometry analysis 24 hours after electroporating the lymphocytes with GFP-encoding EEC harboring UTR variants. (10A) shows graphs of GFP intensity and percentage of Raji lymphocytes transfected with 4 pmoles of GFP-encoding EEC variants. (10B) displays representative graphs of GFP intensity and percentage of cells transfected with 8 pmoles of GFP-encoding EEC variants. (10C) displays bar graphs representing the median GFP intensity {number of experiments=2} of experiments as shown in (10A) and (10B), which show GFP intensity of Raji lymphocytes transfected with 4 and 8 pmoles of GFP-encoding EEC variants, respectively. Data are presented for variants no UTR; Kozak only; 5′ only (SEQ ID NO: 2); 3′ only (SEQ ID NO: 10); 5′ and 3′ ((SEQ ID NO: 2) and (SEQ ID NO: 10)); and Kozak and 3′ (SEQ ID NO: 10).

FIGS. 11A and 11B show the expression of hOCT3/4 protein in HEK293 cells 24 hours after transfection with hOCT3/4-encoding EEC harboring the 5′-3′ UTRs variants disclosed herein. (11A) shows the results from representative flow cytometry experiments indicating the amount of hOCT3/4's intensity and percentage of hOCT3/4 positive HEK293 cells transfected with increasing amount of hOCT3/4-encoding EEC (with full-length 5′ (SEQ ID NO: 2) and 3′ (SEQ ID NO: 10) UTRs). (11B) shows results from representative flow cytometry experiments displaying the amount of hOCT3/4's intensity and percentage of hOCT3/4+ cells transfected with 1.2 pmole of hOCT3/4 with the UTRs variants. The results from the flow cytometry experiment in (11A) are also provided as a line graph. The media results from {number of experiments =2} of experiments as shown in (11B), which show the level of hOCT3/4 with the UTRs variants using the UTR variants when compared to no RNA are also provided as a bar graph.

FIGS. 12A and 12B show the expression of hIL2 protein (as measured by ELISA) from HEK293 cells 24 hours after transfection with hIL2-encoding EEC with the 5′-3′ UTR variants disclosed herein. (12A) displays a graph showing an increase in the absorbance (@450 nm) of ELISA signal with increasing administration of hIL2-encoding EEC {with full-length 5′ (SEQ ID NO: 2) UTR and 3′UTR (SEQ ID NO: 10)}. (12B) displays a bar graph showing relative levels of hIL2 from HEK293 cells transfected with 0.5 pmole of hIL2-encoding EEC with the UTR variants ((SEQ ID NO: 2) and (SEQ ID NO: 10)).

FIGS. 13A-13C show the expression of the GFP protein in HEK293 cells after GFP-encoding EEC transfection with full-length 5′ (SEQ ID NO: 2) UTR and variants of 3′UTR (SEQ ID NOs: 10, 11, and 12). (13A) shows the sequences of the 3′UTR variants. (13B) displays the percent GFP positive HEK293 cells (based on 10,000 events/cells by flow cytometry) 24 hours after transfection of 1-2 pmoles of the various EEC. (13C) is a bar graph displaying the median GFP intensity of HEK293 cells (from two separate experiments) transfected with 1-2 pmoles of GFP-encoding EEC with 3′UTR variants, A, B, and C, as depicted in (13A).

FIG. 14. Oct4 expression in Human Foreskin Fibroblasts upon transfection of engineered Oct4 mRNA constructs (UO: unmodified mRNA Oct4, UMD: unmodified mRNA MyoD-Oct4, PUO: modified mRNA Oct4, PUMD: modified mRNA MyoD-Oct4).

FIG. 15. Additional sequences supporting the disclosure including cDNA constructs to generate in vitro synthesized RNA and resulting synthetic RNA constructs for EGFP, Oct4, and IL2 (SEQ ID NOs: 55-60).

### DETAILED DESCRIPTION

Significant scientific strides in the use of RNA as therapeutics, vaccines and/or to otherwise alter protein expression in cells both in vitro and in vivo have been made. One of the largest problems to overcome is achieving high enough levels of protein expression to allow for a given result. Several attempts have been made to address this problem and increase expression of the target protein to result in mRNA being useful in various clinical contexts. These studies are disclosed in, for example, US20060247195 now abandoned filed on Jun. 8, 2006 assigned to Ribostem Limited; issued U.S. Pat. No. 10,772,975 filed on May 12, 2011 assigned to Moderna; PCT/EP2008/01059 filed on Dec. 12, 2008 published as WO2009077134 assigned to BioNTech AG; PCT/EP2008/03033 filed on Apr. 16, 2008 published as WO2009127230 assigned to Curevac GMBH; PCT/US2016/069079 filed on Dec. 29, 2016 assigned to Cellular Reprogramming, Inc .; and PCT/US2019/037069 filed on Jun. 13, 2019 assigned to Cellular Reprogramming, Inc.

Untranslated regions (UTRs) of a gene are transcribed but not translated. Generally, the 5′ UTR starts at the transcription start site and continues to the start codon but does not include the start codon; the 3′UTR starts following the stop codon and continues until the transcriptional termination signal. Messenger RNAs (mRNAs) include UTRs that are shown to recruit ribosomes, initiate translation and thereby increase protein expression. While according to the preceding description start and stop codons are not generally considered part of UTRs, in the current disclosure, these segments are sometimes included within sequences designated as UTRs to create operational segments.

There is a growing body of evidence about the regulatory roles played by UTRs in terms of stability of nucleic acid molecules and resulting translation/protein expression. Sequences within UTRs differ in prokaryotes and eukaryotes. For example, the Shine-Dalgarno consensus sequence (5′-AGGAGGU-3′) recruits ribosomes in bacteria while the RNA Kozak consensus sequence (5′-GCCRCCRUGG-3′) includes the initiation codon (AUG) and boosts translation initiation events in mammalian cells.

The ‘R’ in the Kozak consensus sequence represents either adenosine or guanosine. The −3 position of the Kozak consensus sequence enhances translation initiation, and as a whole, the Kozak sequence is believed to stall the translation initiation complex for the proper recognition of the start codon. While the Kozak consensus sequence, by itself, can drive ribosomal scanning and translational initiation, additional UTRs associated with highly abundant proteins in the human transcriptome were analyzed. Studies suggest the relative abundance of proteins associated with genetic information processing, including chromosomal and ribosomal associated proteins (Beck et al., The quantitative proteome of a human cell line. Mol. Syst. Biol. (2011), doi: 10.1038/msb.2011.82; Liebermeister et al., Visual account of protein investment in cellular functions. Proc. Natl. Acad. Sci. U. S. A. (2014), doi: 10.1073/pnas.1314810111). For example, the alignment of the 5′UTRs of highly-expressed ribosomal-associated proteins (RPLs/RPSs) illustrate the appearance of the 5′ Terminal OligoPyrimidine Track (i.e. 5′TOP) or C/U (Lavallee-Adam et al., Functional 5′ UTR motif discovery with LESMoN: Local enrichment of sequence motifs in biological networks. Nucleic Acids Res. (2017), doi:10.1093/nar/gkx751; Yoshihama et al., The human ribosomal protein genes: Sequencing and comparative analysis of 73 genes. Genome Res. (2002), doi:10.1101/gr.214202; Cardinali et al., La protein is associated with terminal oligopyrimidine mRNAs in actively translating polysomes. J. Biol. Chem. (2003), doi:10.1074/jbc.M300722200; Pichon et al., RNA Binding Protein/RNA Element Interactions and the Control of Translation. Curr. Protein Pept. Sci. (2012), doi:10.2174/13892031280161947). Generally, 5′TOP sequences are located near the start codon and are important in transcription (i.e. RNA synthesis) and translation of transcripts.

By engineering the features typically found in abundantly expressed genes of specific target organs, one can increase protein expression of the coding sequences. For example, introduction of 5′ UTR of liver-expressed mRNA, such as albumin, serum amyloid A, Apolipoprotein A/B/E, transferrin, alpha fetoprotein, erythropoietin, or Factor VIII, can be used to enhance expression of a coding sequences in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

UTRs, however, can be 100s to 1000s of nucleotides (nts) in length. In light of the use of mRNA in therapeutics and vaccines, the search for minimal/optimal UTRs is favorable for increased and targeted protein expression in cells. In certain examples, engineered expression constructs (EEC) disclosed herein were designed to have minimal UTRs (minUTs). That is the EEC were designed to have 5′ and/or 3′ UTR that are as minimal as possible and still allow for high levels of expression in an intended use.

Thus, according to certain aspects, the current disclosure provides minimal UTRs that dramatically increase protein expression. In certain examples, the current disclosure provides 5′ UTR with 20-23 nucleotides. In certain examples, the current disclosure provides 3′ UTR with 27 nucleotides or 67 nucleotides, depending on whether an optional polyA tail is included. Together then, certain examples of 5′ and 3′ UTR combinations include 47-50 nucleotides or 87-90 nucleotides. These size profiles are beneficial, particularly in therapeutic and/or vaccine applications.

According to certain aspects, to construct minUTs, the current disclosure integrates elements necessary for the production of mRNAs in vitro and protein in vivo. For the production of mRNAs in vitro, the current disclosure employed the use of RNA polymerase from T7 bacteriophage (T7P). T7P binds to a specific DNA double-helix sequence (5′-TAATACGACTCACTATAG-3′ (SEQ ID NO: 45)) and initiates RNA synthesis with the incorporation of guanosine (the last G in the promoter; underlined) as the first ribonucleotide. This binding sequence is generally followed by a pentamer (5′-GGAGA-3′) that serves to stabilize the transcriptional complex, promote T7P clearance and extension of the RNA polymer.

In particular embodiments, 5′ UTRs include a promoter, a mini-enhancer sequence (CAUACUCA, herein), and a Kozak sequence, such as a truncated form of the Kozak sequence (GCCRCC). In particular embodiments, a 5′UTR is described as also being operably linked to a start codon to create an operational segment.

In particular embodiments, minimal promoters are selected for use within a 5′ UTR. Minimal promoters have no activity to drive gene expression on their own but can be activated to drive gene expression when linked to a proximal enhancer element. Exemplary minimal promoters include minBglobin, minCMV, minCMV with a Sacl restriction site removed, minRho, minRho with a Sacl restriction site removed, and the Hsp68 minimal promoter (proHSP68). In particular embodiments, the minimal promoter includes a minimal T7 promoter (mini-T7 promoter).

Certain examples of disclosed 5′ UTR include a unique mini-enhancer sequence (CAUACUCA). The mini-enhancer sequence can be located between a minimal promoter (e.g., T7) and the Kozak consensus sequence to generate a minimal 5′ UTR with 20-23 nucleotides (depending on whether a start codon is designated as part of the UTR). Eukaryotic translation generally starts with the AUG codon, however other start codons can be included. Mammalian cells can also start translation with the amino acid leucine with the help of a leucyl-tRNA decoding the CUG codon and mitochondrial genomes use AUA and AUU in humans. These components and exemplary 5′ UTR are provided in Table 1.

TABLE 1
5′ UTR Components and Constructs..

| SEQ | | |
|---|---|---|
| ID | | |
| NO: | Sequence | Key |
| N/A | CAUACUCA | Mini-enhancer sequence |
| N/A | GGGAGA | Mini-T7 promoter |
| N/A | GCCRCCAUG | Kozak Consensus Sequence |
| 2 | GGGAGACAUA | 5′ UTR Construct with “A” Kozak |
| | CUCAGCCACC | Sequence (no start codon) |
| 3 | GGGAGACAUA | 5′ UTR Construct with “G” Kozak |
| | CUCAGCCGCC | Sequence (no start codon) |
| 38 | GGGAGACAUA | 5′ UTR Construct with “A” Kozak |
| | CUCAGCCACC | Sequence (with start codon) |
| | AUG | |
| 39 | GGGAGACAUA | 5′ UTR Construct with “G” Kozak |
| | CUCAGCCGCC | Sequence (with start codon) |
| | AUG | |

In certain examples, 5′ UTR are capped. For example, eukaryotic mRNAs are guanylylated by the addition of inverted 7-methylguanosine to the 5′ triphosphate (i.e. m7GpppN where N denotes the first base of the mRNA). The m7GpppN or the 5′ cap structure of an mRNA is involved in nuclear export and binds the mRNA Cap Binding Protein (CBP), which is responsible for mRNA translation competency.

The ribose sugars of the first and second nucleotides of mRNAs may optionally also be methylated (i.e. addition of CH3 group) at the 2′-Oxygen (i.e. 2′O) position. A non-methylated mRNA at first and second nucleotides is denoted as Cap0 (i.e. m7GpppN), whereas methylation at the 2′O on the first and second nucleotides are denoted as Cap1 (i.e. m7GpppNm) and Cap2 (i.e. m7GpppNmNm), respectively. Furthermore, if the first 5′ nucleotide is adenosine, it may further be methylated at the 6th Nitrogen (6N) position (i.e. m7Gpppm6A) or form modified Cap0 (i.e. m7Gpppm6A) or modified Cap1 (i.e. m7Gpppm6Am) or modified Cap2 (i.e. m7Gpppm6AmNm).

The RNA guanylylation or the addition of Cap0 (i.e. m7GpppN) may be achieved enzymatically in vitro (i.e. after the RNA synthesis) by Vaccinia Virus Capping Enzyme (VCE). The creation of Cap1 and Cap2 structures may further be achieved enzymatically via the addition of mRNA 2′-O-methyltransferase and S-adenosyl methionine (i.e. SAM). Alternatively, the Cap structure may be added co-transcriptionally in vitro by the incorporation of Anti-Reverse Cap Analog (i.e. ARCA). ARCA is methylated at the 3′-oxygen (3′O) on the cap (m73′OmGpppN) to ensure the incorporation of the cap structure in the correct orientation. In the current application, any of the above cap structures may be used for a final EEC mRNA product.

In particular embodiments, the 5′UTR is operably linked to a coding sequence. As used herein, the term “operably linked” refers to a functional linkage between a nucleotide expression control sequence (e.g., a promoter sequence or a UTR) and another nucleotide sequence, whereby the control sequence allows for and results in the transcription and/or translation of the other nucleotide sequence.

The current disclosure also provides 3′ UTR for optional use with disclosed 5′ UTR. As shown herein, the combination of disclosed 5′ UTR with disclosed 3′ UTR results in EEC with greatly enhanced protein expression over the use of only a disclosed 5′ UTR or only a disclosed 3′ UTR.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA(U/A)(U/A) nonamers.

As indicated previously, disclosed 3′ UTR includes a spacer, a stem loop structure, and optionally, a polyadenine tail (polyA tail). In certain examples herein, the 3′ UTR is also depicted as being operably linked to a stop codon.

Exemplary stop codons include UAA, UGA, and UAG.

Exemplary spacers include [N₁₋₃]AUA and [N₁₋₃]AAA (e.g., UGCAUA, UGCAAA, UGAAA, GCAUA, UAAA, and GAUA), wherein is N is any nucleotide including A,G, C, T, or U. The subscript numbers indicate the quantity of the nucleotide. For example [N₁₋₃] includes 1, 2, or 3 nucleotides as set forth in N, NN, or NNN.

Stem loops (SL), or hairpin or hairpin loops, are a feature of highly expressed transcripts within the 3′UTR. The SLs are distinct secondary structures where complementary nucleotides are paired as the double helix (or the stem) often interrupted with sequences that form the loop. The particular secondary structure represented by the SL includes a consecutive nucleic acid sequence including a stem and a (terminal) loop, also called hairpin loop, wherein the stem is formed by two neighbored entirely or partially complementary sequence elements; which are separated by a short sequence (e.g. 3-10 nucleotides), which forms the loop of the SL structure. The two neighbored entirely or partially complementary sequences may be defined as e.g. SL elements stem 1 and stem 2. The SL is formed when these two neighbored entirely or partially reverse complementary sequences, e.g. SL elements stem 1 and stem 2, form base-pairs with each other, leading to a double stranded nucleic acid sequence including an unpaired loop at its terminal ending formed by the short sequence located between SL elements stem 1 and stem 2. Thus, an SL includes two stems (stem 1 and stem 2), which-at the level of secondary structure of the nucleic acid molecule-form base pairs with each other, and which-at the level of the primary structure of the nucleic acid molecule-are separated by a short sequence that is not part of stem 1 or stem 2. For illustration, a two-dimensional representation of the SL resembles a lollipop-shaped structure. The formation of a stem-loop structure requires the presence of a sequence that can fold back on itself to form a paired double strand; the paired double strand is formed by stem 1 and stem 2. The stability of paired SL elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present invention, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by an SL, the respective complementary nucleic acid sequence is typically also characterized by an SL. An SL is typically formed by single-stranded RNA molecules. In particular embodiments, the SL length is at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length.

The SLs are present within 3′UTR of highly expressed transcripts (e.g. those coding for abundant cellular proteins like histones) where it boosts translation, regardless of polyadenine tail (Gallie et al., The histone 3′-terminal stem-loop is necessary for translation in Chinese hamster ovary cells. Nucleic Acids Res. (1996), doi:10.1093/nar/24.10.1954). The histone 3′UTR stem consensus is characterized as six base-pairs, two of which are G-C pairs, three pyrimidine-purine (Y-R) pairs and one A-U pairs and moreover, the loop includes 4 ribonucleotides with two uridines (U), one purine (Y) and one ribonucleotide (N) (Gallie et al., The histone 3′-terminal stem-loop is necessary for translation in Chinese hamster ovary cells. (Nucleic Acids Res. (1996), doi:10. 1093/nar/24.10.1954; Tan et al., Structure of histone mRNA stem-loop, human stem-loop binding protein, and 3′hExo ternary complex. Science (80). (2013), doi:10.1126/science.1228705; Battle & Doudna, The stem-loop binding protein forms a highly stable and specific complex with the 3′ stem-loop of histone mRNAs. RNA (2001), doi:10.1017/S1355838201001820).

The SLs associate with stem-loop binding proteins (SLBPs) for replication-dependent mRNA stability/processing/metabolism/translation. Structural evidence suggests that the direct contact of SLBPs with SLs occurs at a guanosine nucleotide at the base of SL (G7) (Tan et al., Structure of histone mRNA stem-loop, human stem-loop binding protein, and 3′hExo ternary complex. Science (80). (2013), doi:10.1126/science.1228705; Battle & Doudna, The stem-loop binding protein forms a highly stable and specific complex with the 3′ stem-loop of histone mRNAs. RNA (2001), doi:10.1017/S1355838201001820). Furthermore, the adjacent adenosines, or more specifically, upstream AAA, to the stem impact SLBP binding and function (Battle & Doudna, The stem-loop binding protein forms a highly stable and specific complex with the 3′ stem-loop of histone mRNAs. RNA (2001), doi:10.1017/S1355838201001820; William & Marzluff, The sequence of the stem and flanking sequences at the 3′ end of histone mRNA are critical determinants for the binding of the stem-loop binding protein. Nucleic Acids Res. (1995), doi:10.1093/nar/23.4.654). The 3′UTR also serves to stabilize protein-coding transcripts while increasing their translational capacity. According to certain embodiments, the current disclosure provides designs for synthetic SLs to incorporate the features of SL: three groups of G-C pairs interrupted by a sequence (UAACGGUCUU (SEQ ID NO: 34)) with adjacent spacer sequences such as adenosines to increase SLBP binding and mRNA translation.

Importantly, the stem loops used in EEC are not sequence-orientation dependent and may include a) CCUC and GAGG, b) GAGG and CCUC, c) AAACCUC and GAGG, and d) AAAGAGG and CCUC. Further, the distance between the two arms of the stem (where the CCUC and GAGG base pair) needs to be long enough for a loop to form. In particular embodiments, stem loops can include complementary sequences such as a) RRRR and YYYY, b) RYRR and YRYY, c) RRYR and YYRY, d) RRRY and YYYR, e) RYYR and YRRY, f) RRYY and YYRR, g) YYRR and RRYY, h) YYYR and RRRY, or i) RYYY and YRRR, wherein R is purine (A or G) and Y is a pyrimidine (e.g. U or C).

According to certain embodiments, the number of nucleotides between the two arms may be seven, eight, nine, ten, or longer nucleotides. Preferred embodiments of the length between the two arms of the stem loop are no shorter than seven nucleotides. In certain examples, the loop segment of an SL includes UAACGGUCUU (SEQ ID NO: 34). In particular embodiments, an SL sequence includes GAUGCCCCAUUCACGAGUAGUGGGUAUU (SEQ ID NO: 64), GGCACCCUGCGCAGGUGAUGCAGGUGCC (SEQ ID NO: 65), GUUCGCUCGGUCAGGAGAGCUGACGGAC (SEQ ID NO: 66), UCUUACAGUGGCAUGUGACCGUUUAAGG (SEQ ID NO: 67), CGCGGCGCAUGCACGUGACAUGCCUGCG (SEQ ID NO: 68), CGGUCCCGUGGCAAGAGUCUAUGGAUUG (SEQ ID NO: 69), AUGUUCGGCUCCAAGAGCGAGUUGAUAU (SEQ ID NO: 70), CGAUUCGGGCACAUGUGCUGUCUGAUUG (SEQ ID NO: 71), GUAUUCUGAUGCACGUGCCAUCAAGUAC (SEQ ID NO: 72), or UUGAGCAGGAUCAAGUGCAUUCUUUCAA (SEQ ID NO: 73). In particular embodiments, an SL sequence includes RRYRYYYYRYYYRYRRRYRRYRRRYRYY (SEQ ID NO: 74), RRYRYYYYRYRYRRRYRRYRYRRRYRYY (SEQ ID NO: 75), RYYYRYYYRRYYRRRRRRRYYRRYRRRY (SEQ ID NO: 76), YYYYRYRRYRRYRYRYRRYYRYYYRRRR (SEQ ID NO: 77), YRYRRYRYRYRYRYRYRRYRYRYYYRYR (SEQ ID NO: 78), YRRYYYYRYRRYRRRRRYYYRYRRRYYR (SEQ ID NO: 79), RYRYYYRRYYYYRRRRRYRRRYYRRYRY (SEQ ID NO: 80), YRRYYYRRRYRYRYRYRYYRYYYRRYYR (SEQ ID NO: 81), RYRYYYYRRYRYRYRYRYYRYYRRRYRY (SEQ ID NO: 82), or YYRRRYRRRRYYRRRYRYRYYYYYYYRR (SEQ ID NO: 83), wherein R is purine (A or G) and Y is a pyrimidine (e.g. U or C). See Gorodkin et al., (Nucleic Acids Research 29(10):2135-2144, 2001) for additional exemplary SL motifs.

Optional Poly-A Tails. During natural RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. This process, called polyadenylation, adds a poly-A tail that can be between, for example, 100 and 250 residues long. In in vitro RNA synthesis, a polyA tail may be in encoded on the DNA template and as such is incorporated during the in vitro transcription process.

In certain examples, a polyA tail ranges from 0 to 500 nucleotides in length (e.g., 0, 10, 20, 30, 40, 50, 60, 70, 80, 90, 120, 140, 160, 180, 200, 250, 300, 350, 400, 450, or 500 nucleotides). Certain examples utilize a 40 nucleotide polyA tail. Length may also be determined in units of or as a function of polyA Binding Protein binding. In these embodiments, the polyA tail is long enough to bind 4 monomers of PolyA Binding Protein, 3 monomers of PolyA Binding Protein, 2 monomers of PolyA Binding Protein, or 1 monomer of PolyA Binding Protein. PolyA Binding Protein monomers bind to stretches of 38 nucleotides.

Based on the foregoing discussion of 3′ UTR, 3′ UTR constructs disclosed herein include one or more of spacers (e.g., [N₁₋₃]AUA, [N₁₋₃]AAA, UGCAUA or UGCAAA), stem loop hybridizing sequences (e.g., CCUC, GAGG); stem loop loop segments (e.g., [N₇₋₁₅], UAACGGUCUU (SEQ ID NO: 34)), and/or optionally, polyA tails. When stop codons are designated as part of a 3′ UTR, exemplary stop codons include UAA, UGA, and UAG. Exemplary 3′ UTR constructs based on these components are presented in Table 2.

TABLE 2
3′ UTR Constructs

| SEQ | |
|---|---|
| ID | |
| NO: | Sequence |
| 4 | [N₁₋₃]AUACCUC[N₇₋₁₅]GAGG |
| 5 | [N₁₋₃]AAACCUC[N₇₋₁₅]GAGG |
| 6 | [N₁₋₃]AUAGGAG[N₇₋₁₅]CUCC |
| 7 | [N₁₋₃]AAAGGAG[N₇₋₁₅]CUCC |
| 8 | [N₁₋₃]AUAGAGG[N₇₋₁₅]CCUC |
| 9 | [N₁₋₃]AAAGAGG[N₇₋₁₅]CCUC |
| 10 | Stop-UGCAUACCUCUAACGGUCUUGAGG-PolyA |
| 11 | Stop-UGCAUAGGAGUAACGGUCUUCUCC-PolyA |
| 12 | Stop-UGCAAAGGAGUAACGGUCUUCUCC-PolyA |
| 13 | UAAUGCAUACCUCUAACGGUCUUGAGG |
| 14 | UGAUGCAUACCUCUAACGGUCUUGAGG |
| 15 | UAGUGCAUACCUCUAACGGUCUUGAGG |
| 16 | UAAUGCAUAGAGGUAACGGUCUUCCUC |
| 17 | UGAUGCAUAGAGGUAACGGUCUUCCUC |
| 18 | UAGUGCAUAGAGGUAACGGUCUUCCUC |
| 19 | UAAUGCAAAGAGGUAACGGUCUUCCUC |
| 20 | UGAUGCAAAGAGGUAACGGUCUUCCUC |
| 21 | UAGUGCAAAGAGGUAACGGUCUUCCUC |

In particular embodiments, other non-UTR sequences may be incorporated into the 5′ (or 3′ UTR) UTRs. For example, introns or portions of introns sequences may be incorporated into these regions. Incorporation of intronic sequences may further increase protein expression as well as mRNA levels.

EEC Architectures Utilizing Disclosed 5′ and 3′ UTR. Disclosed engineered sequences for the 5′ UTR and 3′ UTR can be used to create EEC, shown herein to be useful in increasing the protein expression of a variety of proteins when they are used flanking a given coding sequence. These variety of proteins include, green fluorescent protein (GFP), interleukin-2 (IL-2), and POU5F1 (OCT3/4), as disclosed herein. Moreover, these inventive 5′ UTR and 3′ UTR sequences are shown to work similarly in different cell types, including lymphoid and adherent and suspension embryonic kidney cells, irrespective of the manner of transfection.

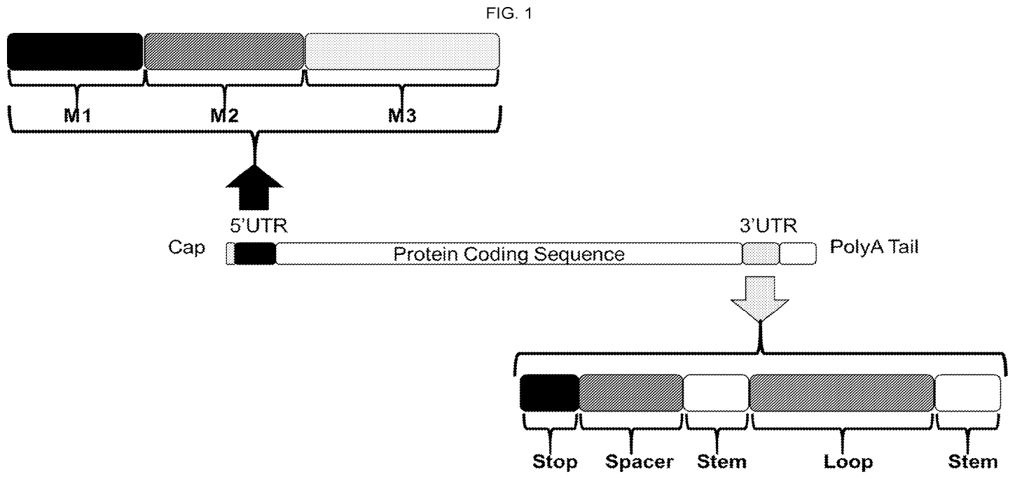

FIG. 1 shows a representative EEC of the present disclosure. In certain examples, “EEC” refers to a polynucleotide transcript having a 5′ and/or 3′ UTR disclosed herein flanking a coding sequence which encodes one or more proteins and which retains sufficient structural and/or chemical features to allow the protein encoded therein to be translated.

Returning to FIG. 1, the depicted EEC includes a coding sequence of linked nucleotides within an open reading frame that is flanked by a first flanking region and a second flanking region. This coding sequence includes an RNA sequence encoding a protein. The protein may include at its 5′ terminus one or more signal sequences encoded by a signal sequence region. The first flanking region may include a region of linked nucleotides including one or more complete or incomplete 5′ UTR sequences. The first flanking region may also include a 5′ terminal cap. Bridging the 5′ terminus of the coding sequence and the first flanking region is a first operational segment. Traditionally this operational segment includes a Start codon. The operational segment may alternatively include any translation initiation sequence or signal including a Start codon.

The first flanking region may include modules that are located within the 5′UTR. This first flanking region may be divided into three modules: module 1 (“M1”), which represents a minimal promoter (e.g., T7 promoter hexamer); module 2 (“M2”) which is a unique translational enhancer (CAUACUCA, described herein); and module 3 (“M3”) which is the Kozak consensus sequence. The T7 promoter hexamer is part of the T7 polymerase promoter, which is in turn part of the T7 class III promoters, a particular class of promoters well known in the art associated with and responsible for inducing the transcription of certain promoters of the T7 bacteriophage. More specifically the T7 promoter and hexamer has the full sequence (5′-TAATACGACTCACTATAGGGAGA-3′ (SEQ ID NO: 31) and initiates RNA synthesis with the incorporation of guanosine as the first ribonucleotide. The Kozak consensus refers to the Kozak consensus sequence (5′-GCCRCCATGG-3′ (SEQ ID NO: 30)) where ‘R’ represents either adenosine or guanosine.

The second flanking region may include a region of linked nucleotides including one or more complete or incomplete 3′ UTRs. The flanking region may also include a 3′ tailing sequence (e.g. polyA tail). The 3′UTR may also divided into three segments including the stop codon, spacer, and a stem-loop segment.

Bridging the 3′ terminus of the coding sequence and the second flanking region is a second operational segment. Traditionally this operational segment includes a Stop codon. The operational segment may alternatively include any translation initiation sequence or signal including a Stop codon. According to the present disclosure, multiple serial stop codons may also be used.

Generally, the shortest length of the coding sequence of the EEC can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 40 amino acids, e.g. no longer than 35, 30, 25, 20, 17, 15, 14, 13, 12, 11 or 10 amino acids. Examples of dipeptides that the polynucleotide sequences can encode or include carnosine and anserine.

Generally, the length of the coding sequence is greater than 30 nucleotides in length (e.g., at least or greater than 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the EEC includes from 30 to 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

According to the present disclosure, the first and second flanking regions may range independently from 5-100 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides).

According to the present disclosure, the capping region may include a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

According to the present disclosure, the first and second operational segments may range from 3 to 40, e.g., 5-30, 10-20, 15, or at least 4, or 30 or fewer nucleotides in length and may include, in addition to a Start and/or Stop codon, one or more signal and/or restriction sequences.

It has been previously attempted to stabilize IVT-RNA by various modifications in order to achieve higher and prolonged expression of transferred IVT-RNA. However, despite the success of RNA transfection-based strategies to express peptides and proteins in cells, there remain issues related to RNA stability, sustained expression of the encoded peptide or protein and cytotoxicity of the RNA. For example, it is known that exogenous single-stranded RNA activates defense mechanisms in mammalian cells.

Several groups have suggested that due to the activated defense mechanisms, to achieve a high enough level of protein expression from IVT-RNA transfected into cells, the mRNA transcript must either contain modified nucleotides (see e.g. issued U.S. Pat. No. 9,750,824 filed on Au. 4, 2012 assigned to University of Pennsylvania) or additional reagents in the form of protein or IVT-RNA that include immune evading factors (see e.g. issued U.S. Pat. No. 10,207,009 filed on May 7, 2015 assigned to BioNTech.) These immune evading factors include viral genes encoding proteins that dampen the cellular immune response by, for example, preventing engagement of the IFN receptor by extracellular IFN (e.g., B18R from vaccinia virus), by inhibiting intracellular IFN signaling (e.g., E3 and K3 both from vaccinia virus) or by working in both capacities (e.g., NS1 from influenza) (Liu et al., Sci Rep 9: 11972, 2019). In particular embodiments, immune evading proteins include B18R, E3, K3, NS1, or ORF8 (from SARS-CoV2).

Aspects of the current disclosure were designed to overcome the activated defense mechanisms by introducing secondary and tertiary structures into the mRNA transcript, instead of using modified nucleotides, microRNAs, or immune evading factors. According to further embodiments, particular embodiments do not use modified nucleotides or microRNAs to increase protein expression. Still further embodiments, do not use modified nucleotides or microRNAs to prolong the translation of from IVT-RNA transfected into cells or for any other purpose.

In certain examples, EEC exclude microRNA binding sites and/or modified NTPs in the 5′ UTR, in the 3′ UTR, in the 5′ UTR and the 3′ UTR, or in the entirety of the EEC.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. In certain examples, EEC do not include any known microRNA target sequences, microRNA sequences, or microRNA seeds.

A microRNA seed is a sequence in the region of positions 2-8 of the mature microRNA, which sequence has perfect Watson-Crick complementarity to the miRNA target sequence.

In certain examples, EEC of the current disclosure are designed to specifically exclude modified NTPs. Modified NTPs are those that have additional chemical groups attached to them to modify their chemical structure. Examples of these modified NTPs include pseudouridine, methylpseudouridine, N1-methyl-pseudouridine, methyluridine (m5U), 5-methoxyuridine (mo5U), and 2-thiouridine (s2U). 5′ caps are not modified NTPs.

In certain examples, EEC include messenger RNA (mRNA). As used herein, “messenger RNA” (mRNA) refers to any polynucleotide which encodes a protein and which is capable of being translated to produce the encoded protein in vitro, in vivo, in situ or ex vivo.

EEC encode proteins or fragments thereof. A “protein” refers to a polymer of amino acid residues (natural or unnatural) linked together most often by peptide bonds. The term includes polypeptides and peptides of any size, structure, or function. In some instances the protein encoded is smaller than 50 amino acids and the protein is then termed a peptide. If the protein is a peptide, it will include at least 2 linked amino acids. Proteins include naturally occurring proteins, synthetic proteins, homologs, orthologs, paralogs, fragments, recombinant proteins, fusion proteins and other equivalents, variants, and analogs thereof. A protein may be a single protein or may be a multi-molecular complex such as a dimer, trimer or tetramer. They may also include single chain or multichain proteins such as antibodies or insulin and may be associated or linked. Most commonly disulfide linkages are found in multichain proteins. The term protein may also apply to amino acid polymers in which one or more amino acid residues are an artificial chemical analogue of a corresponding naturally occurring amino acid.

The term “protein variant” refers to proteins which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least 50% sequence identity to a native or reference sequence, and preferably, they will have at least 80%, or more preferably at least 90% identical sequence identity to a native or reference sequence.

EEC may encode proteins selected from any of several target categories including biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery. Specific proteins may fall into more than one of these categories.

In some embodiments, specific sequences that encode for specific proteins are used. These specific proteins include green fluorescent protein (GFP), interleukin-2 (IL-2), and POU5F1 or OCT ¾ (see FIG. 15). GFP is a protein that exhibits bright green fluorescence when exposed to light. Human POU5F1 or OCT3/4 (herein hOCT4) is a key nuclear transcription factor important in stem cells reprogramming and maintenance. IL-2 is an interleukin, which is a type of cytokine signaling molecule in the immune system. It is a 15.5-16 kDa protein that regulates the activities of leukocytes. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4+ T cells and activated CD8+ T cells.

EEC disclosed herein may encode one or more biologics. “Biologics” include protein that are used to treat, cure, mitigate, prevent, or diagnose a disease or medical condition. Exemplary biologics include allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others.

Antibodies. EEC disclosed herein, may encode one or more antibodies or fragments thereof. The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. As used herein, “monoclonal antibody” refers to an antibody obtained from a population of substantially homogeneous antibodies, i.e., the individual antibodies including the population are identical except for possible naturally occurring mutations and/or post-translation modifications (e.g., isomerizations, amidations) that may be present in minor amounts. Monoclonal antibodies are highly specific, being directed against a single antigenic site.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies herein include “primatized” antibodies including variable domain antigen-binding sequences derived from a non-human primate (e.g., Old World Monkey, Ape etc.) and human constant region sequences.

An “antibody fragment” includes a portion of an intact antibody, preferably the antigen binding and/or the variable region of the intact antibody. Examples of antibody fragments include Fab, Fab′, F(ab′)2 and Fv fragments; diabodies; linear antibodies; nanobodies; single-chain antibody molecules and multispecific antibodies formed from antibody fragments.

Any of the five classes of immunoglobulins, IgA, IgD, IgE, IgG and IgM, may be encoded by coding sequences, including the heavy chains designated alpha, delta, epsilon, gamma and mu, respectively. Also included are polynucleotide sequences encoding the subclasses, gamma and mu. Hence any of the subclasses of antibodies may be encoded in part or in whole and include the following subclasses: IgG1, IgG2, IgG3, IgG4, IgA1 and IgA2.

In particular embodiments, EEC disclosed herein may encode monoclonal antibodies and/or variants thereof. Variants of antibodies may also include substitutional variants, conservative amino acid substitution, insertional variants, deletional variants and/or covalent derivatives. In particular embodiments, the EEC disclosed herein may encode an immunoglobulin Fc region. In another embodiment, the EEC may encode a variant immunoglobulin Fc region. As a non-limiting example, the EEC may encode an antibody having a variant immunoglobulin Fc region.

Particular embodiments encode anti-SARS-Cov2 antibodies, anti-SARS antibodies, anti-RSV antibodies, anti-HIV antibodies, anti-Dengue virus antibodies, anti-Bordatella pertussis antibodies, anti-hepatitis C antibodies, anti-influenza virus antibodies, anti-parainfluenza virus antibodies, anti-metapneumovirus (MPV) antibodies, anti-cytomegalovirus antibodies, anti-Epstein Barr virus antibodies; anti-herpes simplex virus antibodies, anti-Clostridium difficile bacterial toxin antibodies, or anti-tumor necrosis factor (TNF) antibodies.

Known anti-RSV antibodies include palivizumab; those described in U.S. Pat. No. 9,403,900; AB1128 (available from MILLIPORE) and ab20745 (available from ABCAM).

An example of a known anti-HIV antibody is 10E8, which is a broadly neutralizing antibody that binds to gp41. VRC01, which is a broadly neutralizing antibody that binds to the CD4 binding site of gp120. Other exemplary anti-HIV antibodies include ab18633 and 39/5.4A (available from ABCAM); and H81E (available from THERMOFISHER).

Examples of anti-Dengue virus antibodies include antibody 55 (described in U.S. 20170233460); antibody DB2-3 (described in U.S. Pat. No. 8,637,035); and ab155042 and ab80914 (both available from ABCAM).

An anti-pertussis antibody is described in U.S. Pat. No. 9,512,204.

Examples of anti-hepatitis C antibodies include MAB8694 (available from MILLIPORE) and C7-50 (available from ABCAM).

Anti-influenza virus antibodies are described U.S. Pat. No. 9,469,685 and also include C102 (available from THERMOFISHER).

An exemplary anti-MPV antibody includes MPE8.

Exemplary anti-CMV antibodies includes MCMV5322A, MCMV3068A, LJP538, and LJP539. See also, for example, Deng et al., Antimicrobial Agents and Chemotherapy 62(2) e01108-17 (Feb. 2018); and Dole et al., Antimicrobial Agents and Chemotherapy 60(5) 2881-2887 (May 2016).

Examples of anti-HSV antibodies include HSV8-N and MB66.

Exemplary anti-Clostridium difficile antibodies include actoxumab and bezlotoxumab. See also, for example, Wilcox et al., N Engl J Med 376(4) 305-317 (2017).

Numerous additional antibody sequences are available and known to those of ordinary skill in the art that can be used within the teachings of the current disclosure. Sequence information for commercially available antibodies may be found in the Drug Bank database, the CAS Registry, and/or the RSCB Protein Data Bank.

Vaccines. The EEC disclosed herein, may encode one or more vaccines. As used herein, a “vaccine” is a composition that improves immunity to a particular disease or infectious agent by stimulating an immune response to generate acquired immunity against an agent that causes, and/or is necessary to develop, the disease or infection. For example, vaccines are formulations that produce an immune system response against a particular antigen by preemptively exposing the immune system to the antigen. A pathogen antigen can be an intact, but non-infectious form of a pathogen (e.g., heat-killed). Antigens can also be a protein or protein fragment of a pathogen or a protein or protein fragment expressed by an aberrant cell type (e.g. an infected cell or a cancer cell). When the immune system recognizes an antigen following preemptive exposure, it can lead to long-term immune memory so that if the antigen is encountered again, the immune system can quickly and effectively mount an effective response.

Exemplary viral vaccine antigens can be derived from adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses. In particular embodiments, vaccine antigens include peptides expressed by viruses including CMV, EBV, flu viruses, hepatitis A, B, or C, herpes simplex, HIV, influenza, Japanese encephalitis, measles, polio, rabies, respiratory syncytial, rubella, smallpox, varicella zoster, West Nile, and/or Zika.

Examples of vaccine antigens that are derived from whole pathogens include the attenuated polio virus used for the OPV polio vaccine, and the killed polio virus used for the IPV polio vaccine.

As further particular examples, SARS-CoV-02 vaccine antigens include the spike protein or fragments thereof (e.g, the receptor binding domain (RBD)); CMV vaccine antigens include envelope glycoprotein B and CMV pp65; EBV vaccine antigens include EBV EBNAI, EBV P18, and EBV P23; hepatitis vaccine antigens include the S, M, and L proteins of hepatitis B virus, the pre-S antigen of hepatitis B virus, HBCAG DELTA, HBV HBE, hepatitis C viral RNA, HCV NS3 and HCV NS4; herpes simplex vaccine antigens include immediate early proteins and glycoprotein D; human immunodeficiency virus (HIV) vaccine antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; human papillomavirus virus (HPV) viral antigens include the L1 protein; influenza vaccine antigens include hemagglutinin and neuraminidase; Japanese encephalitis vaccine antigens include proteins E, M-E, M-E-NS1, NS1, NS1-NS2A and 80% E; malaria vaccine antigens include the Plasmodium proteins circumsporozoite (CSP), glutamate dehydrogenase, lactate dehydrogenase, and fructose-bisphosphate aldolase; measles vaccine antigens include the measles virus fusion protein; rabies vaccine antigens include rabies glycoprotein and rabies nucleoprotein; respiratory syncytial vaccine antigens include the RSV fusion protein and the M2 protein; rotaviral vaccine antigens include VP7sc; rubella vaccine antigens include proteins E1 and E2; varicella zoster vaccine antigens include gpl and gpll; and zika vaccine antigens include pre-membrane, envelope (E), Domain III of the E protein, and non-structural proteins 1-5.

Additional particular exemplary viral antigen sequences include Nef (66-97): (VGFPVTPQVPLRPMTYKAAVDLSHFLKEKGGL (SEQ ID NO: 48)); Nef (116-145): (HTQGYFPDWQNYTPGPGVRYPLTFGWLYKL (SEQ ID NO: 49)); Gag p17 (17-35): (EKIRLRPGGKKKYKLKHIV (SEQ ID 50)); p17-p24 NO: Gag (253-284): (NPPIPVGEIYKRWIILGLNKIVRMYSPTSILD (SEQ ID NO: 51)); Pol 325-355 (RT 158-188): (AIFQSSMTKILEPFRKQNPDIVIYQYMDDLY (SEQ ID NO: 52)); CSP central repeat region: (NANPNANPNANPNANPNANP (SEQ ID NO: 53)); and E protein Domain III: (AFTFTKIPAETLHTVTEVQYAGTDGPCKVPAQMAVDMQTLTPVGRLITANPVITEGTENSKMML ELDPPFGDSYIVIGVGE (SEQ ID NO: 54)). See Fundamental Virology, Second Edition, eds. Fields, B. N. and Knipe, D. M. (Raven Press, New York, 1991) for additional examples of viral antigens.

In particular embodiments, vaccine antigens are expressed by cells associated with bacterial infections. Exemplary bacteria include anthrax; gram-negative bacilli, chlamydia, diptheria, haemophilus influenza, Helicobacter pylori, Mycobacterium tuberculosis, pertussis toxin, pneumococcus, rickettsiae, Staphylococcus, Streptococcus and tetanus.

As particular examples of bacterial vaccine antigens, anthrax vaccine antigens include anthrax protective antigen; gram-negative bacilli vaccine antigens include lipopolysaccharides; haemophilus influenza vaccine antigens include capsular polysaccharides; diptheria vaccine antigens include diptheria toxin; Mycobacterium tuberculosis vaccine antigens include mycolic acid, heat shock protein 65 (HSP65), the 30 kDa major secreted protein and antigen 85A; pertussis toxin vaccine antigens include hemagglutinin, pertactin, FIM2, FIM3 and adenylate cyclase; pneumococcal vaccine antigens include pneumolysin and pneumococcal capsular polysaccharides; rickettsiae vaccine antigens include rompA; streptococcal vaccine antigens include M proteins; and tetanus vaccine antigens include tetanus toxin.

In particular embodiments, vaccine antigens are derived from multi-drug resistant “superbugs.” Examples of superbugs include Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

Vaccine antigens can also include proteins that are specifically or preferentially expressed by cancer cells in order to activate the immune system to fight cancer. Examples of cancer antigens include A33; BAGE; B-cell maturation antigen (BCMA); Bcl-2; β-catenin; CA19-9; CA125; carboxy-anhydrase-IX (CAIX); CD5; CD19; CD20; CD21; CD22; CD24; CD33; CD37; CD45; CD123; CD133; CEA; c-Met; CS-1; cyclin B1; DAGE; EBNA; EGFR; ephrinB2; estrogen receptor; FAP; ferritin; folate-binding protein; GAGE; G250; GD-2; GM2; gp75, gp100 (Pmel 17); HER-2/neu; HPV E6; HPV E7; Ki-67; L1-CAM; LRP; MAGE; MART; mesothelin; MUC; MUM-1-B; myc; NYESO-1; p53, PRAME; progesterone receptor; PSA; PSCA; PSMA; ras; RORI; survivin; SV40 T; tenascin; TSTA tyrosinase; VEGF; and WT1.

The use of RNA vaccines provides an attractive alternative to circumvent the potential risks of DNA based vaccines. As with DNA, transfer of RNA into cells can also induce both the cellular and humoral immune responses in vivo. In particular, two different strategies have been pursued for immunotherapy with in vitro transcribed RNA (IVT-RNA), which have both been successfully tested in various animal models. Either the RNA is directly injected into the patient by different immunization routes or cells are transfected with IVT-RNA using conventional transfection methods in vitro and then the transfected cells are administered to the patient. RNA may, for example, be translated and the expressed protein presented on the MHC molecules on the surface of the cells to elicit an immune response.

A therapeutic protein refers to a protein that, when expressed by a cell treats an existing medical condition or disorder. “Treats” means that expression of the protein reduces the cause of the existing medical condition or disorder and/or reduces a side effect of the medical condition or disorder (e.g., pain, inflammation, congestion, fatigue, fever, chills).

Cell-Penetrating Proteins. The EEC disclosed herein, may encode one or more cell-penetrating proteins (CPP, also referred to as cell penetrating peptides). A CPP refers to a protein which may facilitate the cellular intake and uptake of molecules. In general, cell penetrating peptides are (short) peptides that are able to transport different types of cargo molecules across the cell membrane, and, thus, facilitate cellular uptake of various molecular cargoes (from nanosize particles to small chemical molecules and large fragments of DNA). Typically, the cargo is associated with the peptides either through chemical linkage via covalent bonds or through non-covalent interactions. Cell-Penetrating peptides are of different sizes, amino acid sequences, and charges, but all CPPs have a common characteristic that is the ability to translocate the plasma membrane and facilitate the delivery of various molecular cargoes to the cytoplasm or to an organelle of a cell. At present, the theories of CPP translocation distinguish three main entry mechanisms: direct penetration in the membrane, endocytosis-mediated entry, and translocation through the formation of a transitory structure (Jafari S, Solmaz M D, Khosro A, 2015, Bioimpacts 5(2): 103-111; Madani F, Lindberg S, Langel L I, Futaki S, Graslund A, 2011 , J Biophys: 414729).

Examples of CPP include Penetratin (Derossi, D., et al., J Biol Chem, 1994. 269(14): p. 10444-50); the minimal domain of TAT required for protein transduction (Vives, E., P. Brodin, and B. Lebleu, J Biol Chem, 1997. 272(25): p. 16010-7); viral proteins, e.g. VP22 (Elliott, C. and P. O'Hare, Cell, 1997. 88(2): p. 223-33) and ZEBRA (Rothe, R., et al., J Biol Chem, 2010. 285(26): p. 20224-33); from venoms, e.g. melittin (Dempsey, C. E., Biochim Biophys Acta, 1990. 1031 (2): p. 143-61), mastoporan (Konno, K., et al., Toxicon, 2000. 38(11): p. 1505-15), maurocalcin (Esteve, E., et al., J Biol Chem, 2005. 280(13): p. 12833-9), crotamine (Nascimento, F. D., et al., J Biol Chem, 2007. 282(29): p. 21349-60) or buforin (Kobayashi, S., et al., Biochemistry, 2004. 43(49): p. 15610-6); or synthetic CPPs, e.g., poly-arginine (R8, R9, R10 and R12) (Futaki, S., et al., J Biol Chem, 2001. 276(8): p. 5836-40) or transportan (Pooga, M., et al., FASEB J, 1998. 12(1): p. 67-77).

A CPP may contain one or more detectable labels. The proteins may be partially labeled or completely labeled throughout. The EEC may encode the detectable label completely, partially or not at all. The cell-penetrating peptide may also include a signal sequence. As used herein, a “signal sequence” refers to a sequence of amino acid residues bound at the amino terminus of a nascent protein during protein translation. The signal sequence may be used to signal the secretion of the cell-penetrating polypeptide.

The CPP encoded by the EEC may form a complex after being translated. The complex may include a charged protein linked to the cell-penetrating polypeptide.

In particular embodiments, the CPP may include a first domain and a second domain. The first domain may include a supercharged polypeptide. The second domain may include a protein-binding partner. As used in this context, a “protein-binding partner” includes antibodies and functional fragments thereof, scaffold proteins, or peptides. The CPP may further include an intracellular binding partner for the protein-binding partner. The CPP may be capable of being secreted from a cell where the EEC was introduced. The CPP may also be capable of penetrating the cell in which the EEC was introduced.

In a further embodiment, the CPP is capable of penetrating a second cell. The second cell may be from the same area as the first cell, or it may be from a different area. The area may include tissues and organs. The second cell may also be proximal or distal to the first cell.

In particular embodiments, the EEC may also encode a fusion protein. A fusion protein includes at least two domains that are not present together in a naturally occurring protein. The domains can be directly fused or can be connected through an intervening linker sequence. In certain examples, a fusion protein includes a charged protein linked to a therapeutic protein. A “charged protein” refers to a protein that carries a positive, negative or overall neutral electrical charge. Preferably, the therapeutic protein may be covalently linked to the charged protein in the formation of the fusion protein. The ratio of surface charge to total or surface amino acids may be 0.1, 0.2, 0.3, 0.4, 0.5, 0.6, 0.7, 0.8 or 0.9. Other examples of fusion proteins include bi-specific antibodies, chimeric antigen receptors, and engineered T cell receptors (TCR).

Secreted Proteins. Human and other eukaryotic cells are subdivided by membranes into many functionally distinct compartments. Each membrane-bounded compartment, or organelle, contains different proteins essential for the function of the organelle. The cell uses “sorting signals,” which are amino acid motifs located within the protein, to target proteins to particular cellular organelles.

One type of sorting signal, called a signal sequence, a signal peptide, or a leader sequence, directs a class of proteins to an organelle called the endoplasmic reticulum (ER). Proteins targeted to the ER by a signal sequence can be released into the extracellular space as a secreted protein. Similarly, proteins residing on the cell membrane can also be secreted into the extracellular space by proteolytic cleavage of a “linker” holding the protein to the membrane.

In some embodiments, EEC can be used to manufacture large quantities of human gene products.

In some embodiments, EEC can be used to express a protein of the plasma membrane.

In some embodiments, EEC can be used to express a cytoplasmic or cytoskeletal protein.

In some embodiments, EEC can be used to express an intracellular membrane bound protein.

In some embodiments, EEC can be used to express a nuclear protein.

In some embodiments, EEC can be used to express a protein associated with human disease.

In some embodiments, EEC can be used to express a protein with a presently unknown therapeutic function.

In certain examples, EEC encode one or more proteins currently being marketed or in development. Incorporation of the encoding polynucleotide of a protein currently being marketed or in development into an EEC can result in increased protein expression as described herein.

EEC can encode more than one protein by including within the coding sequence a coding sequence for a self-cleaving peptide or by including a ribosomal skipping element.

Proteins encoded by EEC may be utilized to treat conditions or diseases in many therapeutic areas such as blood, cardiovascular, CNS, poisoning (including antivenoms), dermatology, endocrinology, gastrointestinal, medical imaging, musculoskeletal, oncology, immunology, respiratory, sensory and anti-infective.

When used to treat a subject, EEC can be formulated for administration.

Formulations of EEC may be prepared by any method known or hereafter developed in the art of pharmacology. In general, such preparatory methods include the step of bringing the EEC into association with an excipient and/or one or more other accessory ingredients, and then, if necessary and/or desirable, dividing, shaping and/or packaging the formulation into desired single- or multi-dose units.

Relative amounts of the EEC, the pharmaceutically acceptable excipient, and/or any additional ingredients in a formulation in accordance with the disclosure will vary, depending upon the identity, size, and/or condition of the subject treated and further depending upon the route by which the formulation is to be administered. By way of example, the formulation may include between 0.1% and 100%, e.g., between 0.5 and 50%, between 1-30%, between 5-80%, at least 80% (w/w) active ingredient.

EEC formulations can include one or more excipients to: (1) increase stability; (2) increase cell transfection; (3) permit sustained or delayed release (e.g., from a depot formulation); (4) alter biodistribution (e.g., target to specific tissues or cell types); and/or (5) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients can also include lipidoids, liposomes, lipid nanoparticles, polymers, lipoplexes, core-shell nanoparticles, peptides, proteins, cells transfected with EEC (e.g., for transplantation into a subject), hyaluronidase, nanoparticle mimics and combinations thereof.

In vitro Synthesis of EEC.

The process of mRNA production may include in vitro transcription, cDNA template removal and RNA clean-up, and mRNA capping and/or tailing reactions.

During in vitro transcription, cDNA from a desired construct is produced according to techniques well known in the art. This given cDNA may be transcribed using an in vitro transcription (IVT) system. This IVT may allow for in-vitro synthesized mRNA of disclosed EEC. The system typically includes a transcription buffer, nucleotide triphosphates (NTPs), an RNase inhibitor and a polymerase. The NTPs may be manufactured in house, may be selected from a supplier, or may be synthesized as known in the art. The NTPs are selected from naturally occurring NTPs. The polymerase may be selected from T7 RNA polymerase, T3 RNA polymerase, SP6 RNA polymerase and mutant polymerases such as polymerases able to incorporate modified nucleic acids.

Transfection of the EEC into Mammalian Cells. EEC designed and synthesized as described herein may then be transfected into a variety of cell types, wherein the encoded protein within the open reading frame will be translated into the protein of interest. Transfection may occur using any known method in the art, for example, electroporation and lipofection. The variety of cell types includes any mammalian cell that is known or may become known in the art. Examples of mammalian cells that may be used include Jurkat, Raji, HEK293, primary fibroblast, primary blood cells (including a variety of white blood cells), primary kidney cells, primary liver cells, primary pancreatic cells and primary neurons.

The present disclosure provides EEC including an in vitro-synthesized RNA which includes a coding sequence within an open reading frame for translation in a mammalian cell. The protein may be selected from a wide variety of proteins, including those that will reside in the cytoplasm, will be transported to an organelle, and will be secreted. These EEC may include a 5′ UTR including any one of the sequence CAUACUCA, SEQ ID NO: 2, SEQ ID NO: 3, SEQ ID NO: 38, or SEQ ID NO: 39 or a 3′ UTR including SEQ ID NOs: 4, 5, 6, or 7. The 5′ UTR may also include a T7 polymerase promoter, a mini-enhancer sequence (CAUACUCA), or a Kozak sequence. Further, the bacteriophage T7 promoter may be selected from a T7 Class III promoters (SEQ ID NO: 2) in the engineered sequences.

The present disclosure also provides EEC for an in vitro-synthesized RNA including a coding sequence within an open reading frame for translation in a mammalian cell, where the EEC may also include a 3′ UTR including SEQ ID NOs: 4, 5, 6, or 7 in conjugation with either stop codons (UAA/UAG/UGA). Further, the in vitro-synthesized mRNA may also include a 3′ UTR including either a) CCUC and GAGG or b) GAGG and CCUC. Either set of sequences (a) CCUC and GAGG or b) GAGG and CCUC) of the 3′ UTR sequences may be separated by no fewer than seven nucleotides or may be greater than seven nucleotides. Preferentially, the total number of nucleotides in the 3′ UTR sequences may be no more than fifty nucleotides.

The present disclosure provides EEC and methods for the engineered in vitro-synthesized mRNA may include a 5′ UTR including any one of the mini-enhancer sequence (CAUACUCA) SEQ ID NO: 2, SEQ ID NO: 3, SEQ ID NO: 38, or SEQ ID NO: 39 and a 3′ UTR including of one of SEQ ID NOs: 4, 5, 6, or 7. Further, the engineered in vitro-synthesized mRNA may include a 5′ UTR including the mini-enhancer sequence (CAUACUCA), SEQ ID NO: 2, SEQ ID NO: 3, SEQ ID NO: 38, or SEQ ID NO: 39 and a 3′ UTR including of one of SEQ ID NOs: 4, 5, 6, or 7. Moreover, the engineered in vitro-synthesized mRNAs may include any one of the mini-enhancer sequence (CAUACUCA)SEQ ID NO: 2, SEQ ID NO: 3, SEQ ID NO: 38, or SEQ ID NO: 39 and one of SEQ ID NOs: 4, 5, 6, or 7. Furthermore, EEC of the current disclosure may include engineered mRNA with a coding sequence encoding any one of Green Fluorescent Protein (GFP), Human Interleukin-2 (IL2) and Human POU5F1 (or OCT3/4). The present disclosure also provides EEC where the in vitro-synthesized RNA increases the expression level of the encoded protein. Further, in certain examples, EEC of the current disclosure do not include any modified nucleosides and/or do not include any microRNA binding sites. In additional examples, EEC of the current disclosure do not include any modified nucleosides, do not include any microRNA binding sites, and do not include any immune-evading agents.

As described herein, EEC increase expression of a protein. This increase can be in relation to natural expression levels of a protein, when compared to coding sequences that do not include the mini-enhancer sequence in the 5′ UTR, when compared to coding sequences that do not include the stem-loop sequence in the 3′ UTR, when compared to coding sequences that do not include the mini-enhancer sequence in the 5′ UTR and the stem-loop sequence in the 3′ UTR, when compared to coding sequences that contain modified nucleotides, but not the EEC disclosed herein, and/or in relation to how a protein has been historically or conventionally expressed. In certain examples, the increased protein expression is at least 10% more protein expression, at least 20% more protein expression, at least 30% more protein expression, at least 40% more protein expression, at least 50% more protein expression, at least 60% more protein expression, at least 70% more protein expression, at least 80% more protein expression, at least 90% more protein expression, at least 100% more protein expression, at least 200% more protein expression, at least 300% more protein expression as compared to a relevant control system or condition.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. An engineered expression construct (EEC) having a 5′ untranslated region (UTR) operably linked to a coding sequence, wherein the 5′ UTR has the sequence as set forth in CAUACUCA in between a minimal promoter and a Kozak sequence.

2. The EEC of embodiment 1, wherein the minimal promoter is a T7 promoter.

3. The EEC of embodiment 2, wherein the T7 promoter has the sequence as set forth in GGGAGA.

4. The EEC of any of embodiments 1-3, wherein the Kozak sequence has the sequence as set forth in GCCRCCAUG, wherein R is A or G.

5. The EEC of any of embodiments 1-4, wherein the 5′ UTR has
(i) the sequence as set forth in SEQ ID NO: 2 operably linked to a start codon or
(ii) the sequence as set forth in SEQ ID NO: 3 operably linked to a start codon.

6. The EEC of embodiment 5, wherein the sequence as set forth in SEQ ID NO: 2 operably linked to a start codon has the sequence as set forth in SEQ ID NO: 38.

7. The EEC of embodiment 5, wherein the sequence as set forth in SEQ ID NO: 3 operably linked to a start codon has the sequence as set forth in SEQ ID NO: 39.

8. The EEC of any of embodiments 1-7, wherein the 5′ UTR is less than 30 nucleotides.

9. The EEC of any of embodiments 1-8, further including a 3′ UTR.

10. The EEC of embodiment 9, wherein the 3′ UTR includes a spacer, and a stem loop structure operably linked to a stop codon.

11. The EEC of embodiment 10, wherein the stop codon has the sequence UAA, UGA, or UAG.

12. The EEC of embodiments 10 or 11, wherein the spacer has the sequence [N₁₋₃]AUA or [N₁₋₃]AAA.

13. The EEC of embodiments 10 or 11, wherein the spacer has the sequence UGCAUA or UGCAAA.

14. The EEC of embodi any of embodiments ment 10-13, wherein the stem loop structure has hybridizing sequences as set forth in CCUC and GAGG.

15. The EEC of any of embodiments 10-13, wherein the stem loop structure has hybridizing sequences as set forth in AAACCUC and GAGG or as set forth in AAAGAGG and CCUC.

16. The EEC of any of embodiments 10-15, wherein the stem loop structure has a loop segment having at least 7 nucleotides.

17. The EEC of any of embodiments 10-16, wherein the stem loop structure has a loop segment having 7-15 nucleotides.

18. The EEC of any of embodiments 10-17, wherein the stem loop structure has a loop segment having the sequence as set forth in UAACGGUCUU (SEQ ID NO: 34).

19. The EEC of any of embodiments 9-18, wherein the 3′ UTR is less than 30 nucleotides.

20. The EEC of any of embodiments 9-19, wherein the 3′ UTR further includes a polyadenine (polyA) tail.

21. The EEC of embodiment 20, wherein the polyA tail has 60 residues or less.

22. The EEC of embodiments 20 or 21, wherein the polyA tail has 40 residues.

23. The EEC of any of embodiments 9-22, wherein the 3′ UTR has the sequence as set forth in SEQ ID NO: 4, 5, 6, 7, 8, or 9.

24. The EEC of any of embodiments 9-22, wherein the 3′ UTR has the sequence as set forth in SEQ ID NO: 10, 11, or 12.

25. The EEC of any of embodiments 9-24, wherein the 3′ UTR has the sequence as set forth in SEQ ID NO: 13, 14, 15, 16, 17, 18, 19, 20, or 21.

26. The EEC of any of embodiments 1-25, wherein the EEC includes in vitro-synthesized messenger RNA (mRNA).

27. The EEC of any of embodiments 1-26, wherein the coding sequence encodes Green Fluorescent Protein (GFP), Human Interleukin-2 (IL2) or Human POU5F1 (or OCT3/4).

28. The EEC of any of embodiments 1-27, having the sequence as set forth in SEQ ID NO: 56, 58, or 60.

29. The EEC of any of embodiments 1-26, wherein the coding sequence encodes a therapeutic protein.

30. The EEC of embodiment 29, wherein the therapeutic protein includes an antibody or binding fragment thereof.

31. The EEC of embodiment 30, wherein the antibody or binding fragment thereof includes an anti-SARS-Cov2 antibody or binding fragment thereof, an anti-SARS antibody or binding fragment thereof, an anti-RSV antibody or binding fragment thereof, an anti-HIV antibody or binding fragment thereof, an anti-Dengue virus antibody or binding fragment thereof, an anti-Bordatella pertussis antibody or binding fragment thereof, an anti-hepatitis C antibody or binding fragment thereof, an anti-influenza virus antibody or binding fragment thereof, an anti-parainfluenza virus antibody or binding fragment thereof, an anti-metapneumovirus (MPV) antibody or binding fragment thereof, an anti-cytomegalovirus antibody or binding fragment thereof, an anti-Epstein Barr virus antibody; anti-herpes simplex virus antibody or binding fragment thereof, an anti-Clostridium difficile bacterial toxin antibody or binding fragment thereof, or an anti-tumor necrosis factor (TNF) antibody or binding fragment thereof.

32. The EEC of any of embodiments 1-26, wherein the coding sequence encodes a vaccine antigen.

33. The EEC of embodiment 32, wherein the vaccine antigen includes a SARS-CoV-02 vaccine antigen, a CMV vaccine antigen, an EBV vaccine antigen, a hepatitis vaccine antigen, a herpes simplex vaccine antigen, a human immunodeficiency virus (HIV), vaccine antigen, a human papillomavirus virus (HPV) viral antigen, an influenza vaccine antigen, a Japanese encephalitis vaccine antigen, a malaria vaccine antigen, a measles vaccine antigen, a rabies vaccine antigen, a respiratory syncytial vaccine antigen, a rotaviral vaccine antigen, a varicella zoster vaccine antigen, or a zika vaccine antigen.

34. The EEC of any of embodiments 1-26, wherein the coding sequence encodes a cytokine.

35. The EEC of any of embodiments 1-26, wherein the coding sequence encodes a cell-penetrating protein.

36. Thee EEC of embodiment 35, wherein the cell-penetrating protein includes penetratin, the minimal domain of TAT, VP22, ZEBRA, melittin, mastoporan, maurocalcin, crotamine, buforin, poly-arginine, or transportan.

37. The EEC of any of embodiments 1-36, wherein the EEC does not include modified nucleosides.

38. The EEC of any of embodiments 1-37, wherein the EEC does not include microRNA binding sites.

39. The EEC of any of embodiments 1-38, wherein the coding sequence encodes an immune evading factor.

40. The EEC of embodiment 40, wherein the immune evading factor includes B18R, E3, K3, NS1, or ORF8.

41. The EEC of any of embodiments 1-38, wherein the EEC does not include immune evading factors.

42. The EEC of any of embodiments 1-41, formulated for administration to a subject.

43. An engineered expression construct (EEC) having a coding sequence operably linked to a 5′ untranslated region (UTR) including the sequence as set forth in SEQ ID NO:38 and a 3′ UTR including the sequence as set forth in SEQ ID NO: 13, 14, or 15.

44. An enhancer sequence including the sequence as set forth in CAUACUCA.

45. An engineered expression (EEC) construct having 1, 2, 3, 4, or 5 copies of the sequence as set forth in CAUACUCA.

46. The EEC of embodiment 44, wherein the enhancer sequence is operably linked to a promoter.

47. The EEC of embodiment 46, wherein the promoter is a minimal promoter.

48. An engineered expression construct including an in vitro-synthesized RNA including a coding sequence within an open reading frame that encodes a protein for translation in a mammalian cell, wherein said in vitro-synthesized RNA further includes one of a 5′ untranslated region including CAUACUCA and a 3′ untranslated region including one of SEQ ID NOs: 4, 5, 6, or 7.

49. An engineered expression construct including an in vitro-synthesized RNA including a coding sequence within an open reading frame that encodes a protein for translation in a mammalian cell, wherein said in vitro-synthesized RNA further includes one of a 5′ untranslated region including SEQ ID NO: 2, or SEQ ID NO: 3 and a 3′ untranslated region including SEQ ID NOs: 4, 5, 6, or 7.

50. An engineered expression construct including an in vitro-synthesized RNA including an open reading frame that encodes a protein for translation in a mammalian cell, wherein said in vitro-synthesized RNA further includes a 5′ untranslated region including a T7 polymerase promoter, the sequence as set forth in CAUACUCA, and a Kozak sequence.

51. The engineered expression construct of any of embodiments 1-42, wherein the T7 promoter is selected from a T7 Class III promoter.

52. An engineered expression construct including an in vitro-synthesized RNA including an open reading frame that encodes a protein for translation in a mammalian cell, wherein said in vitro-synthesized RNA includes a 3′ untranslated region including SEQ ID NOs: 4, 5, 6, or 7 and a stop codon.

53. The engineered expression construct of embodiment 52, wherein the stop codon is UAA, UAG, or UGA.

54. An engineered expression construct including an in vitro-synthesized RNA including an open reading frame that encodes a protein for translation in a mammalian cell, wherein said in vitro-synthesized RNA includes a 3′ untranslated region including either a) CCUC and GAGG or b) GAGG and CCUC, wherein either set of the 3′ untranslated region sequences is separated by no fewer than seven nucleotides.

55. An engineered expression construct (EEC) including an in vitro-synthesized RNA including an open reading frame that encodes a protein for translation in a mammalian cell, wherein said in vitro-synthesized RNA includes a 3′ untranslated region including either a) AAACCUC and GAGG or b) AAAGAGG and CCUC, wherein either set of the 3′ untranslated region sequences is separated by no fewer than seven nucleotides.

56. A 5′ untranslated region (UTR) including a sequence as set forth in CAUACUCA that is in between a minimal promoter and a Kozak sequence.

57. The 5′UTR of embodiment 56, wherein the minimal promoter is a T7 promoter.

58. The 5′UTR of embodiment 57, wherein the T7 promoter has the sequence as set forth in GGGAGA.

59. The 5′UTR of any of embodiments 56-58, wherein the Kozak sequence has the sequence as set forth in GCCRCCAUG, wherein R is adenosine or guanine.

60. The 5′UTR of any of embodiments 56-59, including the sequence as set forth in SEQ ID NO: 2 operably linked to a start codon.

61. The 5′UTR of any of embodiments 56-60, including the sequence as set forth in SEQ ID NO: 3 operably linked to a start codon.

62. The 5′UTR of embodiment 60, wherein the sequence as set forth in SEQ ID NO: 2 operably linked to a start codon has the sequence as set forth in SEQ ID NO: 38.

63. The 5′UTR of embodiment 61, wherein the sequence as set forth in SEQ ID NO: 3 operably linked to a start codon has the sequence as set forth in SEQ ID NO: 39.

64. The 5′UTR of any of embodiments 56-63, wherein the 5′ UTR is less than 30 nucleotides.

65. A 3′UTR including a spacer and a stem loop structure operably linked to a stop codon, wherein the stop codon has the sequence UAA, UGA, or UAG and the spacer has the sequence [N₁₋₃]AUA or [N₁₋₃]AAA.

66. The 3′UTR of embodiment 65, wherein the spacer has the sequence UGCAUA or UGCAAA.

67. The 3′UTR of embodiments 65 or 66, wherein the stem loop structure includes hybridizing sequences as set forth in CCUC and GAGG.

68. The 3′UTR of any of embodiments 65-67, wherein the stem loop structure includes hybridizing sequences as set forth in AAACCUC and GAGG or as set forth in AAAGAGG and CCUC.

69. The 3′UTR of any of embodiments 65-68, wherein the stem loop structure has a loop segment having at least 7 nucleotides.

70. The 3′UTR of any of embodiments 65-69, wherein the stem loop structure has a loop segment having 7-15 nucleotides.

71. The 3′UTR of any of embodiments 65-70, wherein the stem loop structure has a loop segment having the sequence as set forth in UAACGGUCUU (SEQ ID NO: 34).

72. The 3′UTR of any of embodiments 65-71, wherein the 3′ UTR is less than 30 nucleotides.

73. The 3′UTR of any of embodiments 65-72, wherein the 3′ UTR further includes a polyadenine (polyA) tail.

74. The 3′UTR of embodiment 73, wherein the polyA tail has 60 residues or less.

75. The 3′UTR of embodiments 73 or 74, wherein the polyA tail has 40 residues.

76. The 3′UTR of any of embodiments 65-75, wherein the 3′ UTR has the sequence as set forth in SEQ ID NO: 4, 5, 6, 7, 8, or 9.

77. The 3′UTR of any of embodiments 65-75, wherein the 3′ UTR has the sequence as set forth in SEQ ID NO: 10, 11, or 12.

78. The 3′UTR of any of embodiments 65-77, wherein the 3′ UTR has the sequence as set forth in SEQ ID NO: 13, 14, 15, 16, 17, 18, 19, 20, or 21.

79. The 3′UTR of any of embodiments 65-78, wherein the 3′UTR is operably linked to a coding sequence.

80. The 3′UTR of embodiment 79, wherein the coding sequence encodes a therapeutic protein, a vaccine antigen, a cytokine, or a fluorescent protein.

81. The 3′UTR of embodiments 79 or 80, wherein the coding sequence encodes an immune evading factor.

82. The 3′UTR of embodiment 81, wherein the immune evading factor includes B18R, E3, K3, NS1, or ORF8.

83. The 3′UTR of any of embodiments 79-80, wherein the coding sequence does not include an immune evading factor.

84. The 3′UTR of any of embodiments 79-83, wherein the coding sequence encodes a cell-penetrating protein.

85. The 3′UTR of embodiment 84, wherein the cell-penetrating protein includes penetratin, the minimal domain of TAT, VP22, ZEBRA, melittin, mastoporan, maurocalcin, crotamine, buforin, poly-arginine, or transportan.

86. A 5′ UTR and/or 3′ UTR sequence as disclosed herein.

87. A 5′ UTR and/or 3′ UTR sequence as disclosed herein operably linked to a coding sequence.

Experimental Examples. Example 1. Materials and Methods. UTR Design and Structure Prediction. The minimal transcription and translation elements (for example, the unique 5′UTR enhancer, the T7 hexamer, and the Kozak sequence, all described herein), which are four to ten nucleotides in length, are assembled to construct the UTRs of the current disclosure. Based on the stem loop features, synthetic 3′ sequences were assembled for testing. The secondary structure prediction webservers (rna.urmc.rochester.edu/RNAstructureWeb/) were utilized with default parameters to examine the likelihood of stem loop secondary structure being formed from the various UTR sequences.

table 1
List of PCR Primers

| Universal PCR | |
|---|---|
| Primer ID | Sequence |
| Forward (No | GACTCCTAATACGACTCACTA |
| UTR) | TAGGGAGAATG |
| | (SEQ ID NO: 24) |
| Forward | GACTCCTAATACGACTCACTA |
| (Kozak) | TAGGGAGAGCCACCATG |
| | (SEQ ID NO: 25) |
| Forward | GACTCCTAATACGACTCACTA |
| (5′UTR) | TAGGGAGACATACTCAGCCAC |
| | CATG |
| | (SEQ ID NO: 26) |
| Reverse | TTTTTTTTTTTTTTTTTTTTT |
| (3′UTR- | TTTTTTTTTTTTTTTTTTTCC |
| A) | TCAAGACCGTTAGAGGTATGC |
| | A (SEQ ID NO: 27) |
| Reverse | TTTTTTTTTTTTTTTTTTTTT |
| (3′UTR-B) | TTTTTTTTTTTTTTTTTTTGG |
| | AGAAGACCGTTACTCCTATGC |
| | A (SEQ ID NO: 28) |
| Reverse | TTTTTTTTTTTTTTTTTTTTT |
| (3′UTR-C) | TTTTTTTTTTTTTTTTTTTGG |
| | AGAAGACCGTTACTCCTTTGC |
| | A (SEQ ID NO: 29) |

mRNA Synthesis. DNA fragments from Integrated DNA Technologies Inc. (IDT) were used in polymerase chain reactions (PCRs) to construct T7 promoter, the UTRs, and the polyadenosine (40 adenosines) sequences by the oligonucleotides shown in Table 1. The templates were then used in the transcription reactions with T7 RNA polymerase, Anti-Reverse Cap Analog (ARCA) to synthesize mRNAs (HiScribe™ T7 ARCA mRNA Kit, NEB). Following DNase I treatment, the mRNAs were quantified and stored accordingly.

Cell Cultures. HEK293 (ATCC® CRL-1573™), Jurkat, clone E6-1 (ATCC® TIB-152™) and Raji (ATCC® CCL-86™) cells were obtained from the American Type Culture Collection (ATCC). All cells were maintained at 37° C. with 5% CO₂. HEK293 media includes Eagle's Minimum Essential Medium (EMEM) (ATCC® 30-2003™) with 10% fetal bovine serum (FBS). Jurkat and Raji cells are maintained in RPMI-1640 Medium (ATCC® 30-2001™) supplemented with 10% FBS. Expi293™ Expression System Kit (ThermoFisher) was used according to the manufacturer's instructions.

Transfection and Electroporation. For optimal transfection parameters, cells were transfected with increasing levels of EEC including full-length UTRs. For comparative studies, HEK293 cells were transfected with 0.4-1 pmole of mRNAs mixed with MessengerMax lipofectamine (Thermo Fisher Scientific, Inc.). Jurkat and Raji cells were transfected with 0-16 pmoles of GFP-encoding EEC mixed with jetMessenger (PolyPlus). For electroporation, Neon Transfection System (Thermo Fisher Scientific, Inc.) were used according to the manufacturer's manual. The database was referred to for the optimal electroporation parameters (voltage, duration and number of pulses).

Flow Cytometry. Cells were fixed with 4% paraformaldehyde for 30-60 minutes and stored in phosphate buffered saline (PBS). For hOCT3/4 staining, cells were permeabilized, incubated with antibodies (BioLegend), washed two times and stored in PBS until use. Cells were analyzed on FACSCalibur (BD) and CytoFlex (Beckman Coulter) flow cytometers and analyzed on the FlowJo software.

ELISA. Following 24 hours after transfection, cell media were collected, spun and diluted accordingly. Human IL2 ELISA (BioLegend) were used to quantitate expression according to the manufacturer's protocol. Briefly, plates (Costar) were coated with capture antibodies, followed by incubation with the diluted cell media, detection antibody and avidin-HRP. Absorbance (450 nm) were read and analyzed.

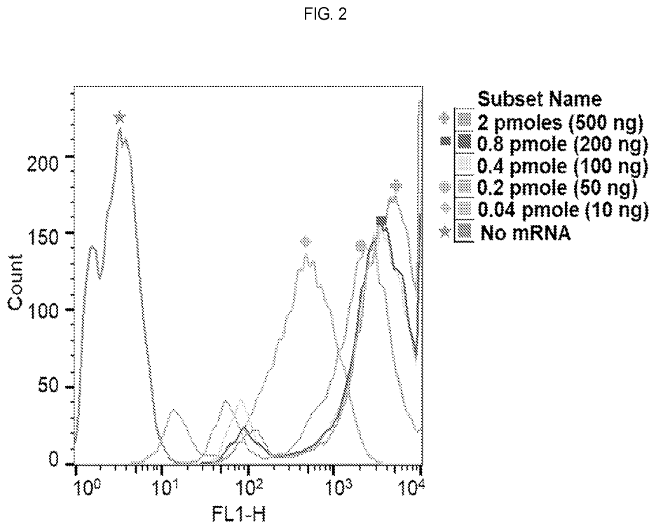

Example 2. EEC containing the disclosed unique 5′UTR sequences resulted in increased protein expression when compared to no 5′UTR sequences. To test the EEC sequence's ability to increase protein expression, EEC containing the modified 5′UTR and 3′UTR sequences with GFP as a reporter protein were transfected into EXPI293 suspension cells (Thermo Fisher Scientific, Inc.). EXPI293 suspension cells derived from the HEK293 cell line were utilized initially, because they are designed for high protein expression. To determine the appropriate amount of mRNA to use in the experiments, EXPI293 cells were transfected with increasing levels of GFP-encoding EEC, ranging from 0-2 pmoles (0-500 ng) using the EXPIfectamine transfection reagent. After 24 hours, the cells were subjected to flow cytometry, as described above. In these experiments, the GFP fluorescent signal is considered proportional to its protein levels in cells. As shown in FIG. 2, the Flow cytometry data suggested GFP median intensity saturates at 0.4 pmole (100 ng) of mRNA per 6.0×10⁵ cells.

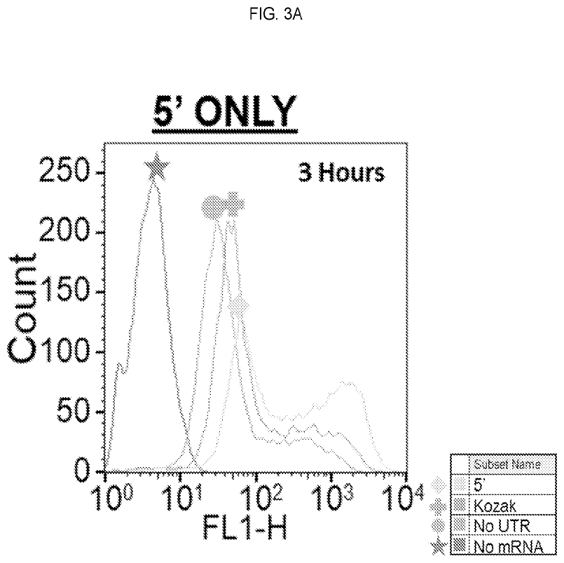

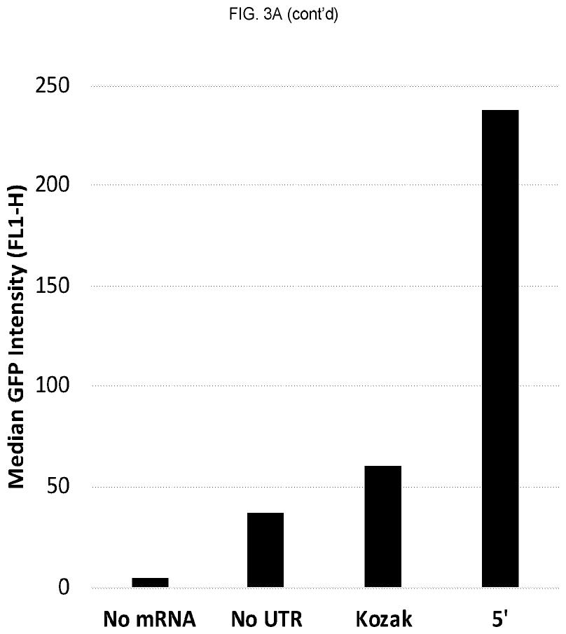

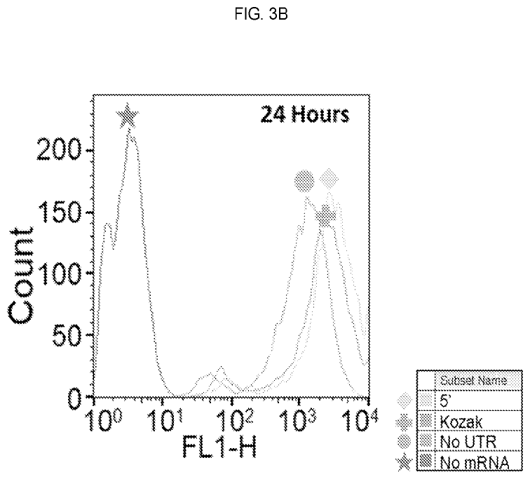

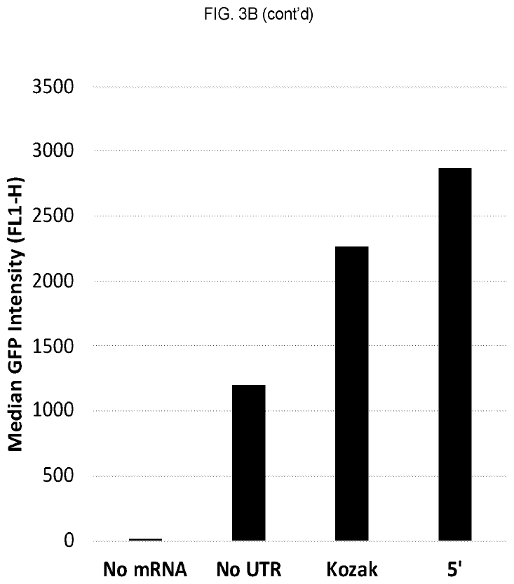

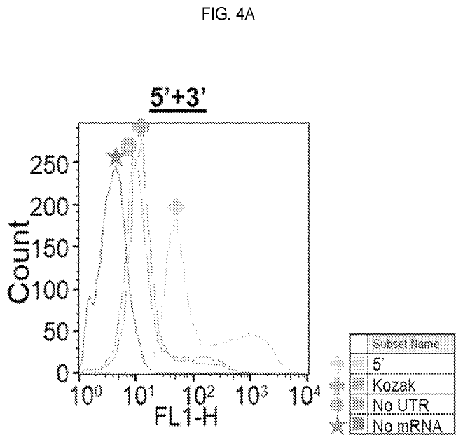

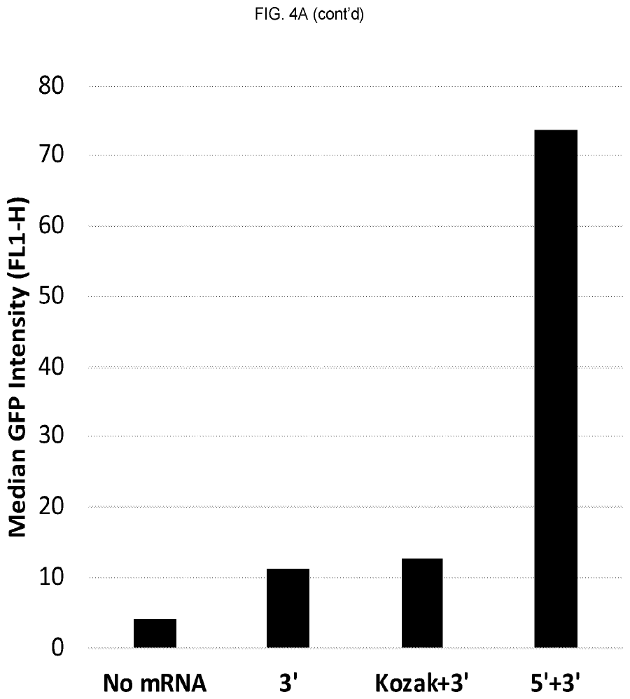

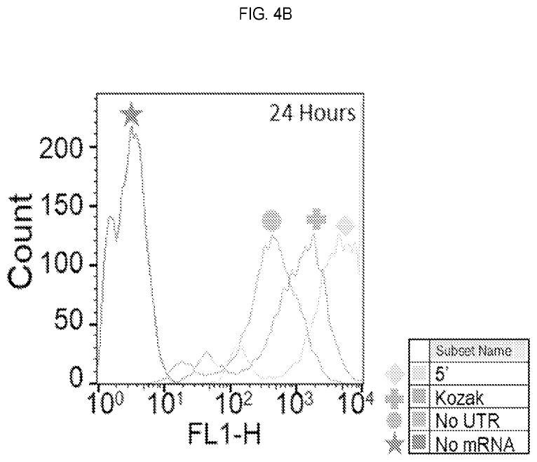

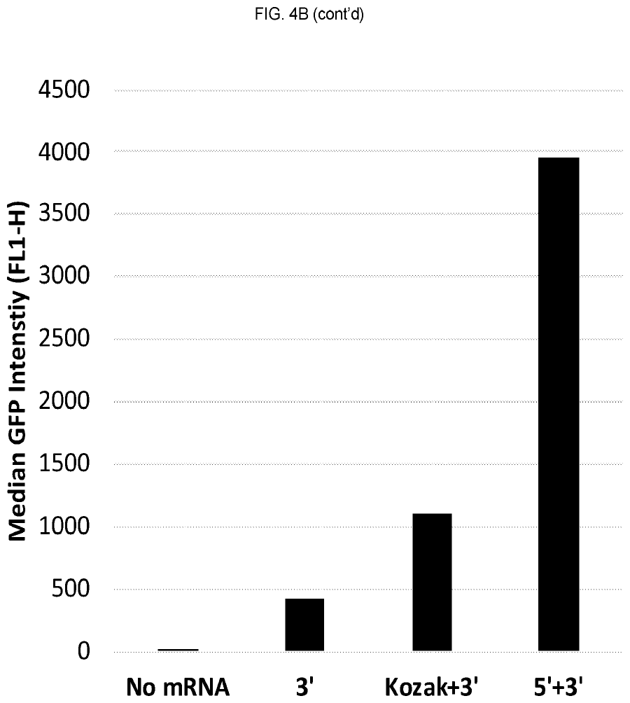

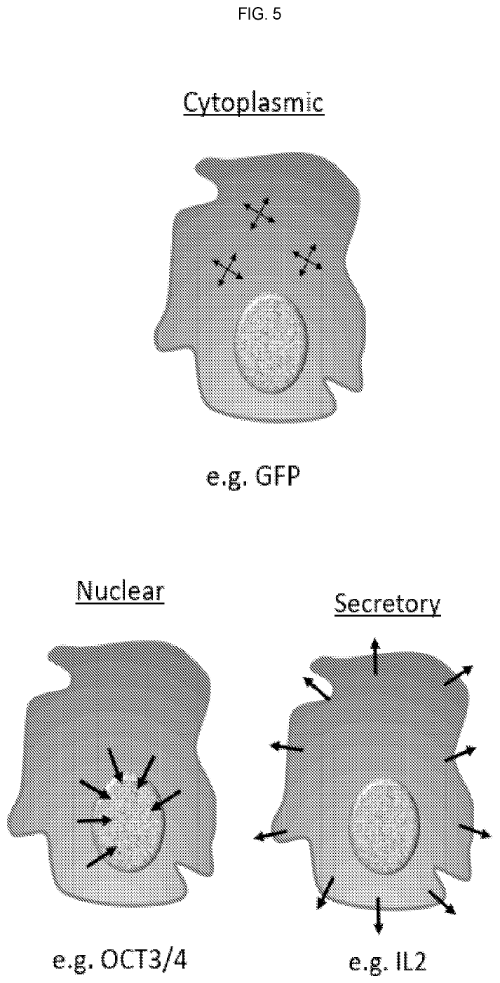

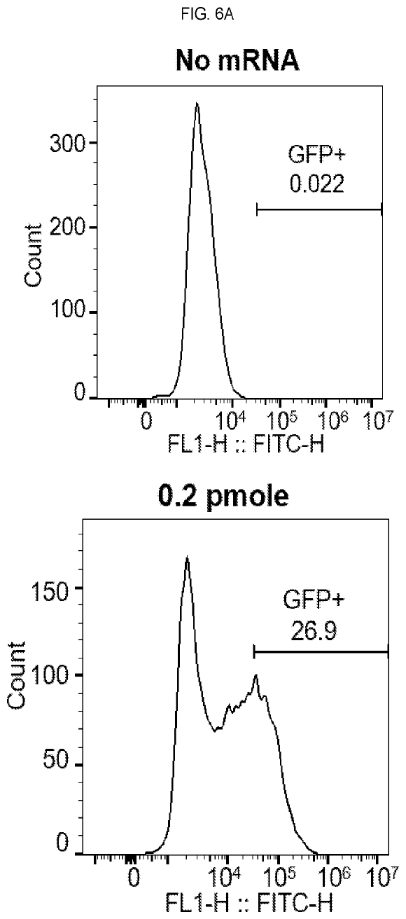

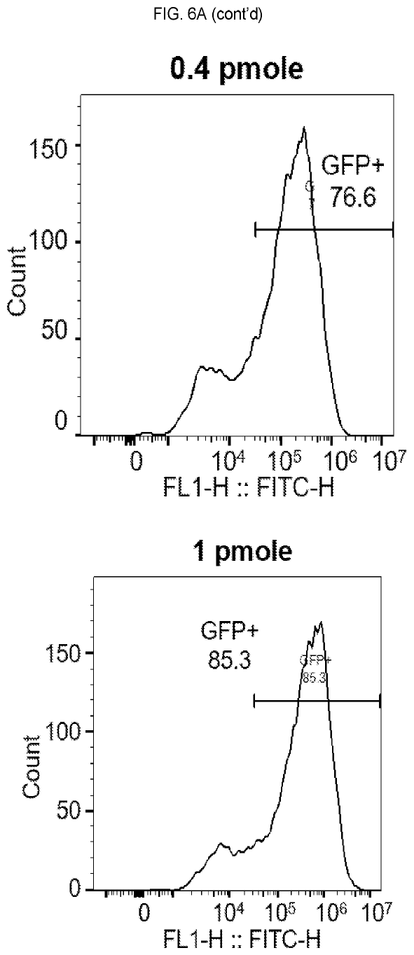

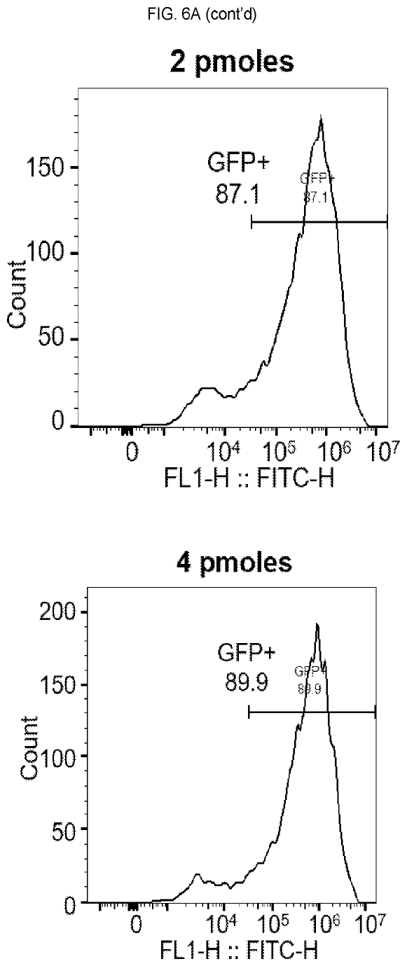

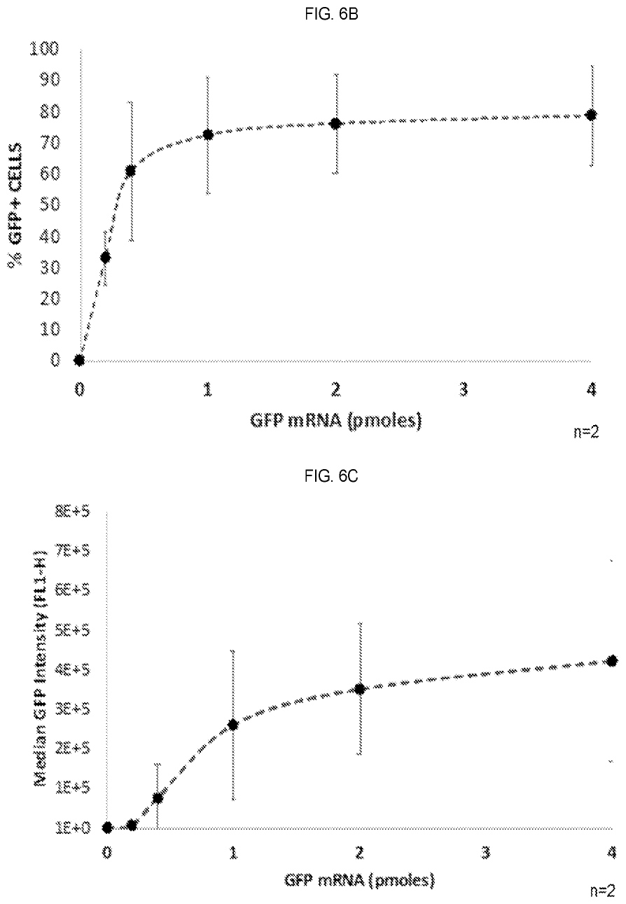

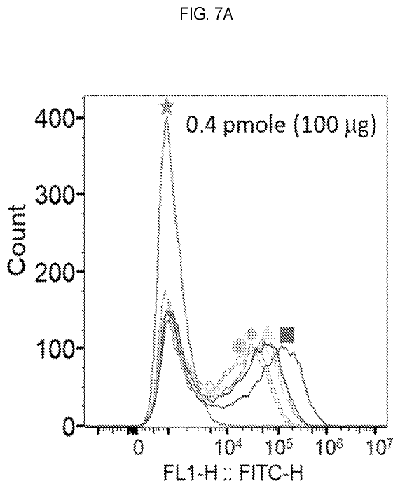

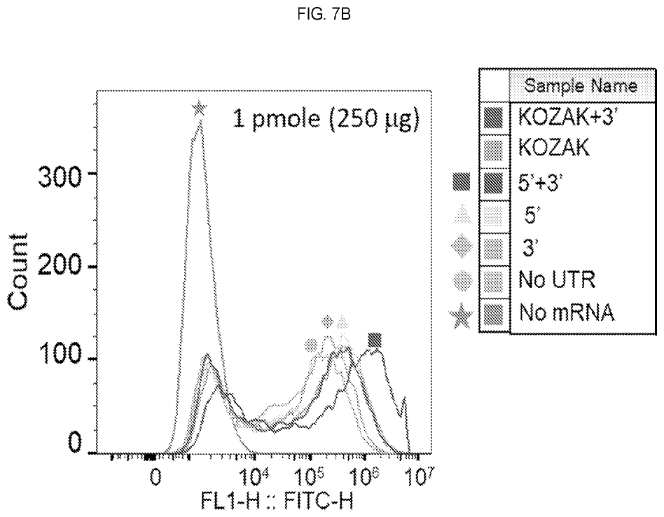

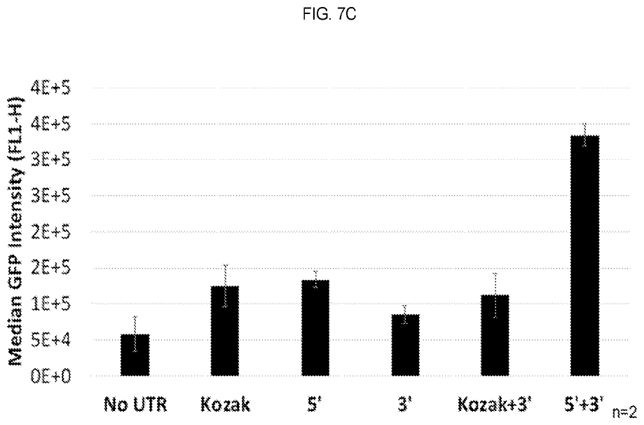

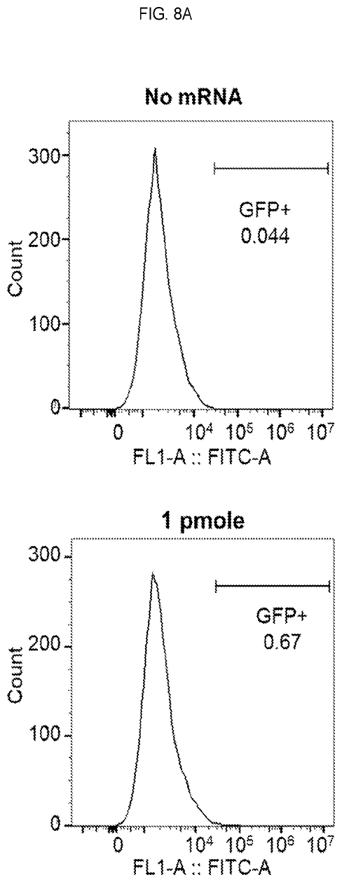

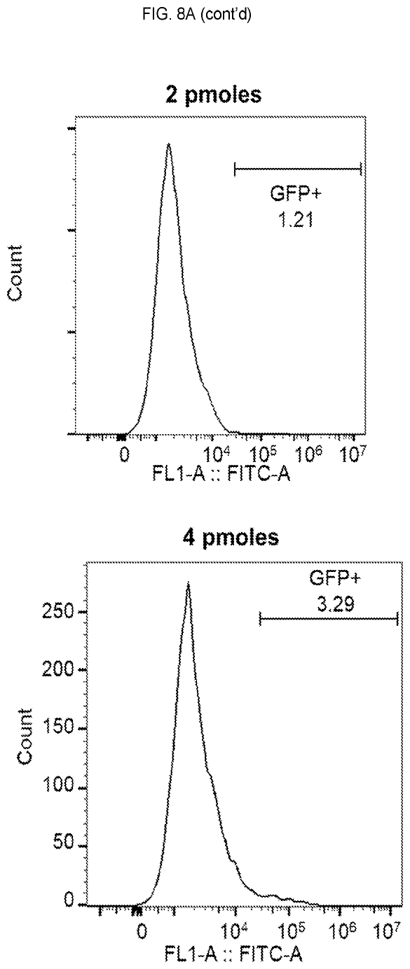

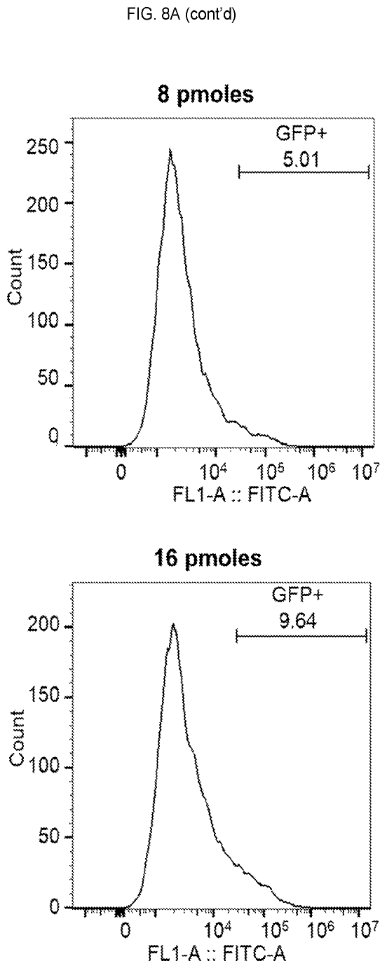

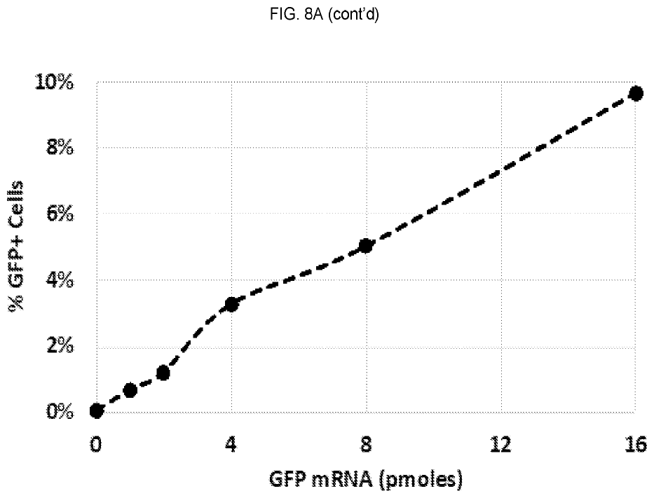

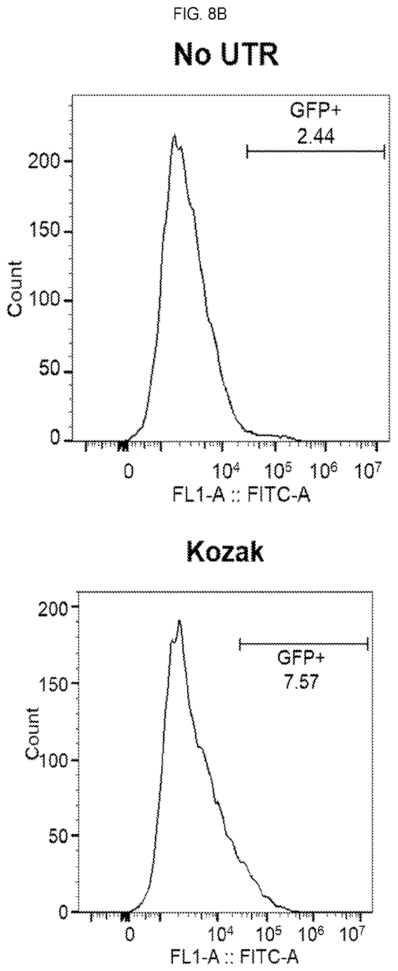

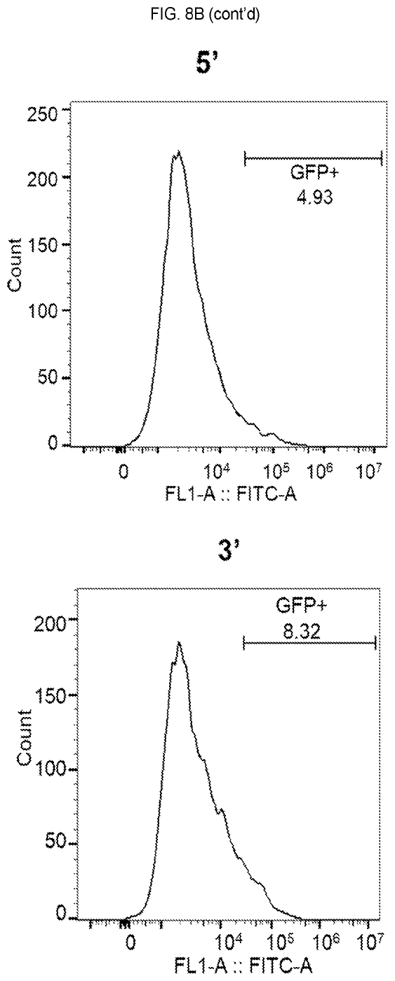

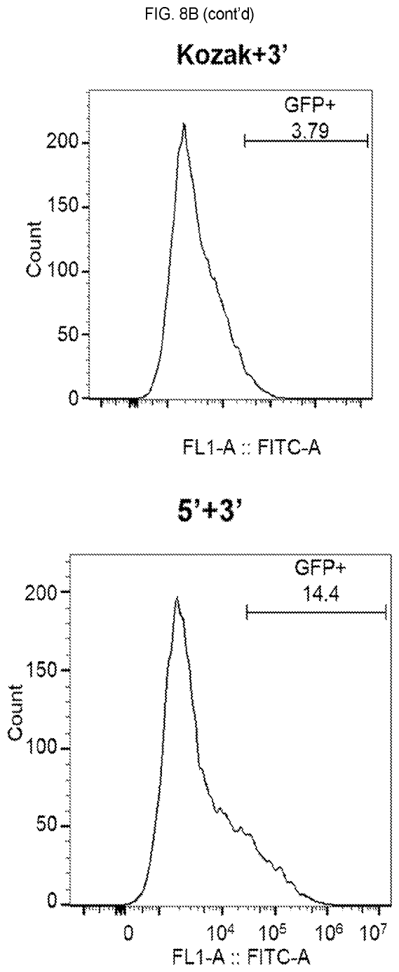

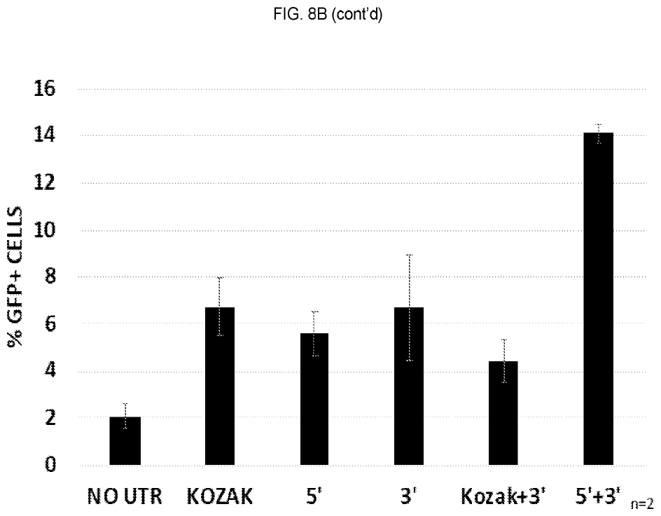

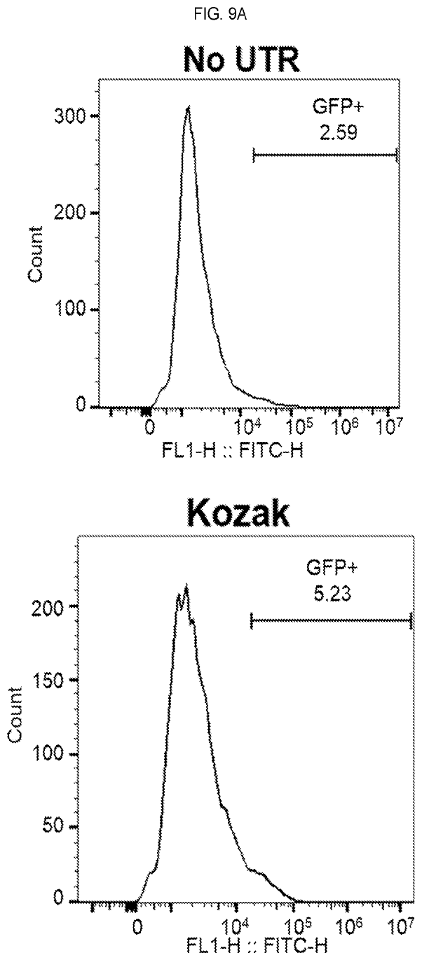

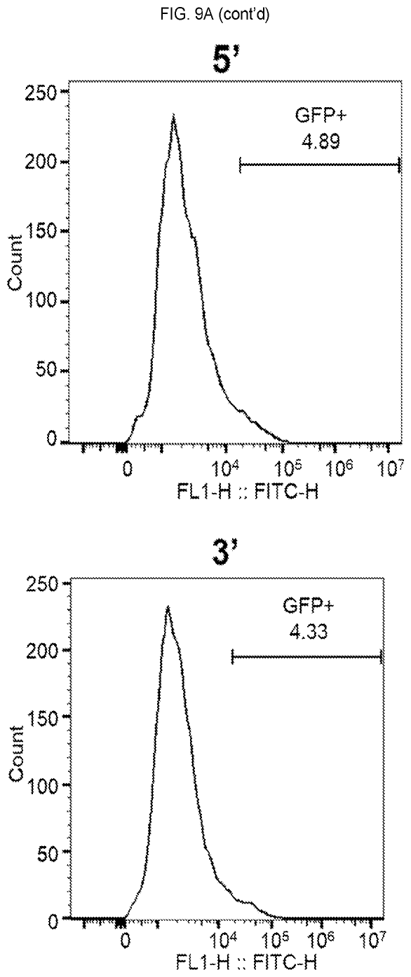

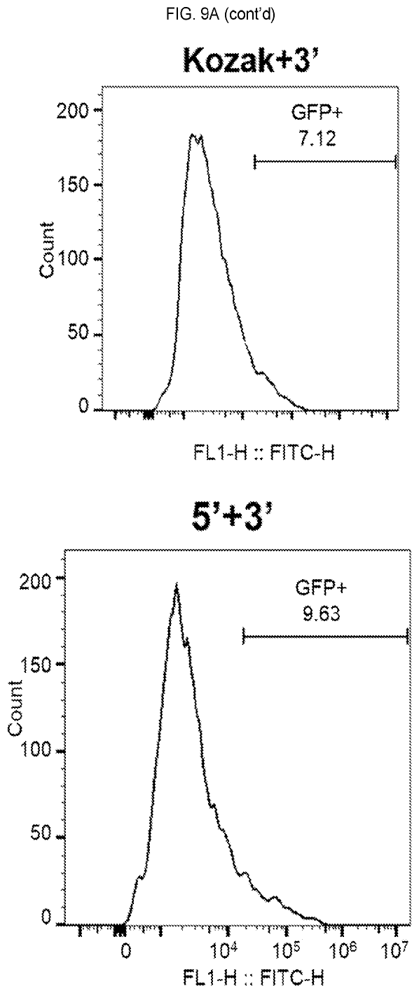

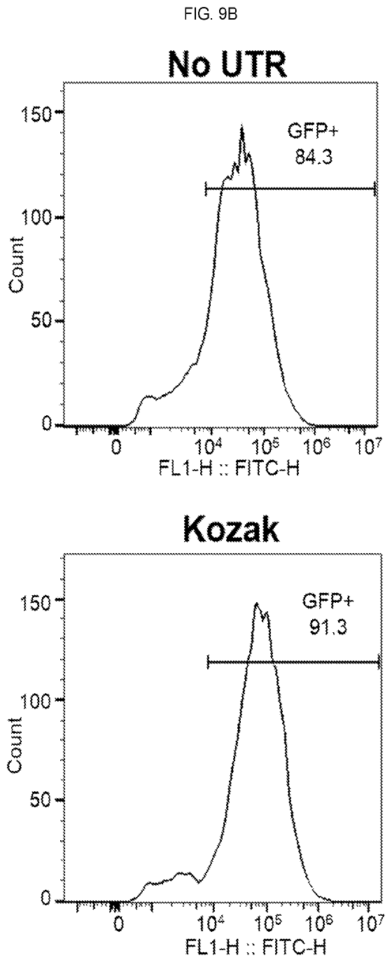

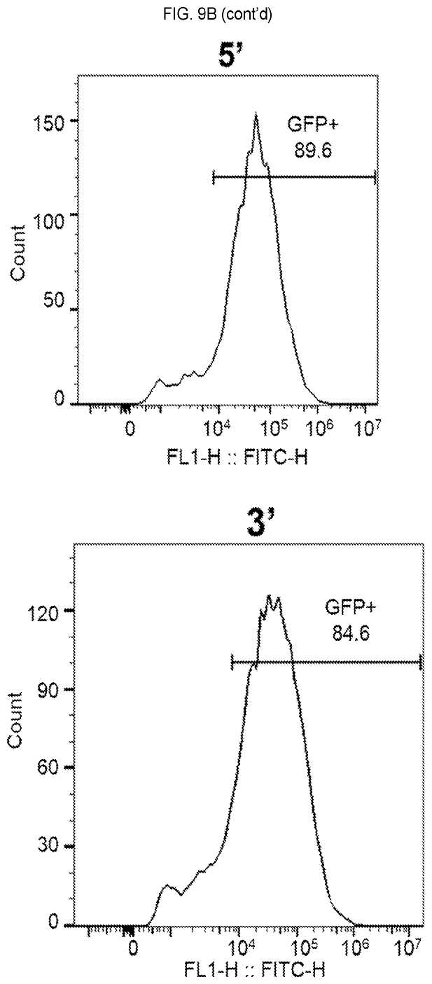

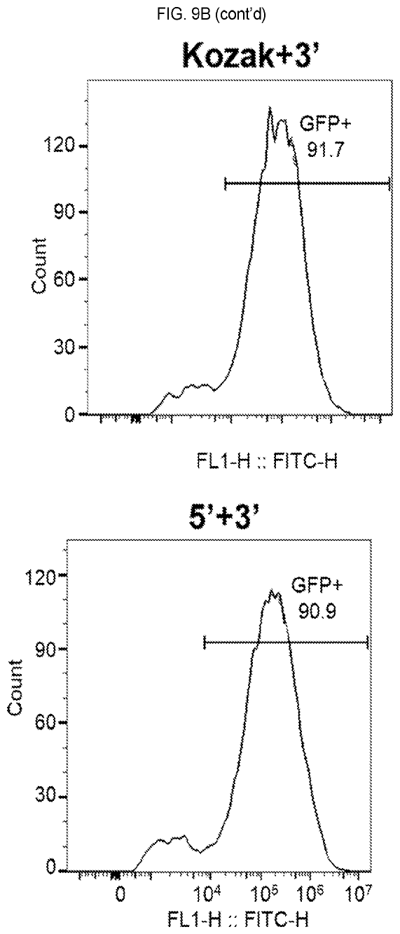

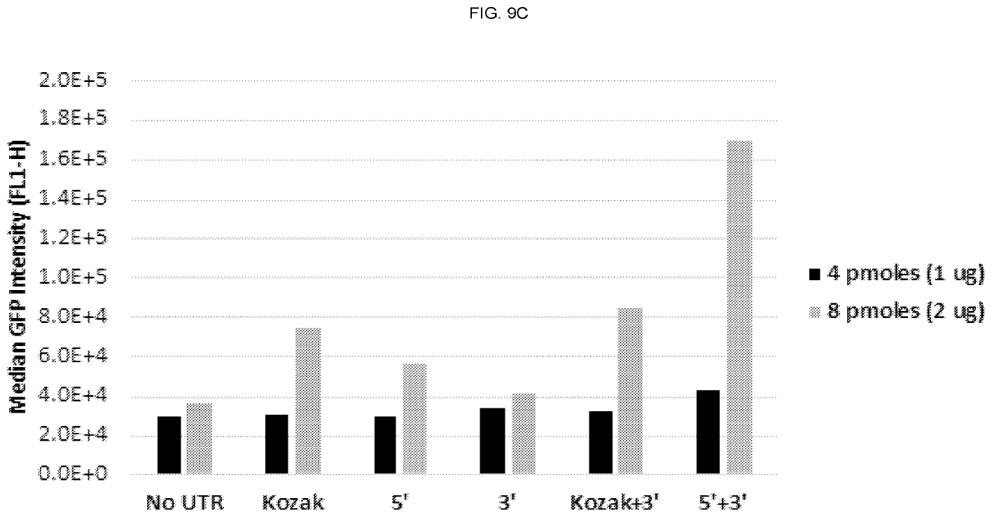

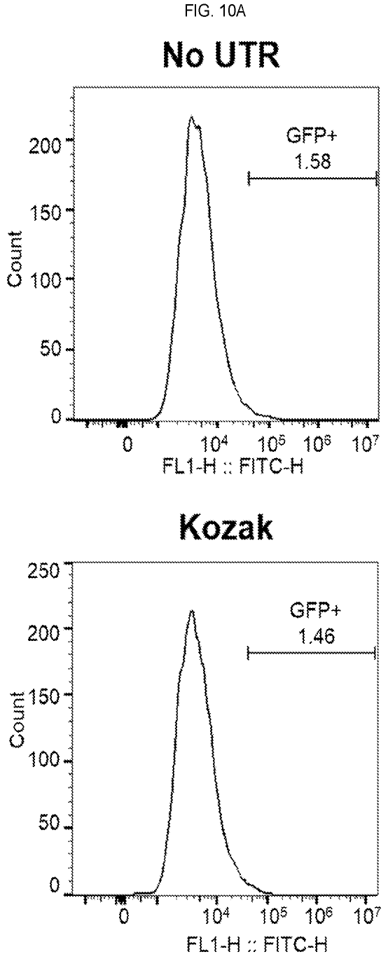

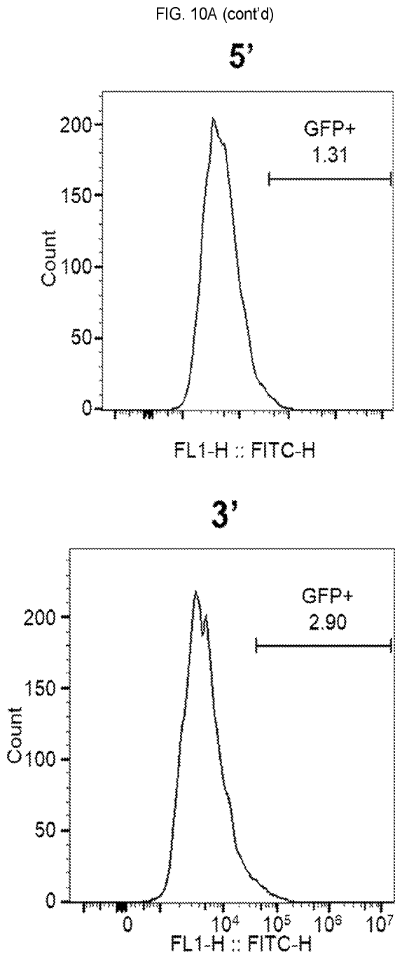

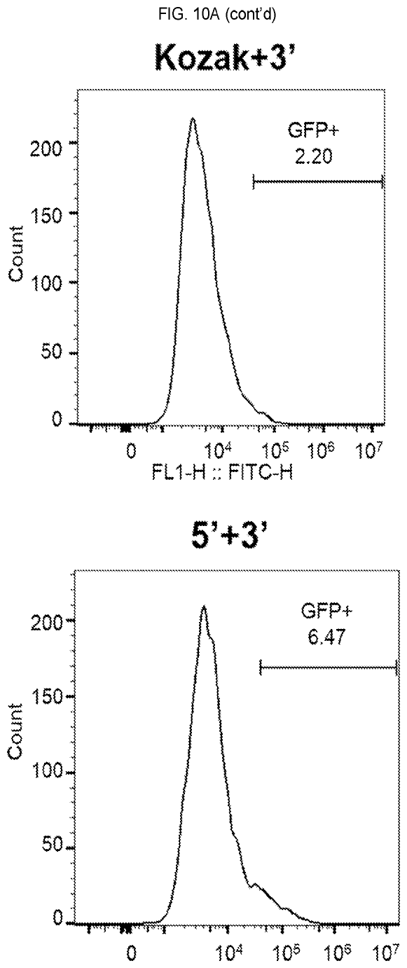

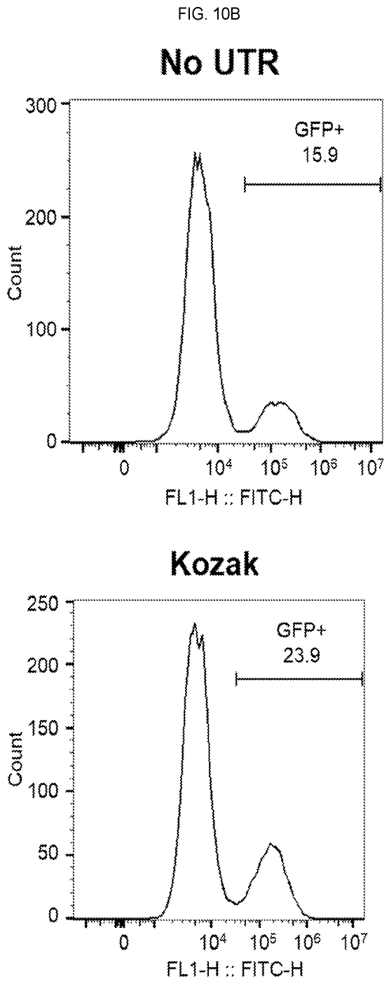

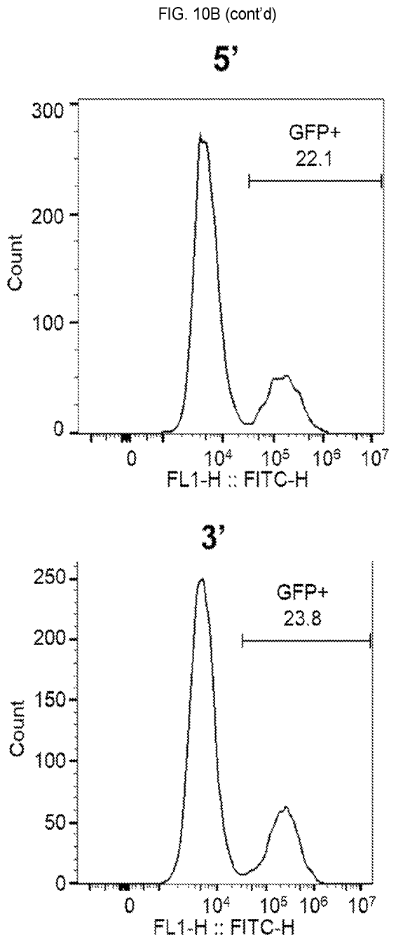

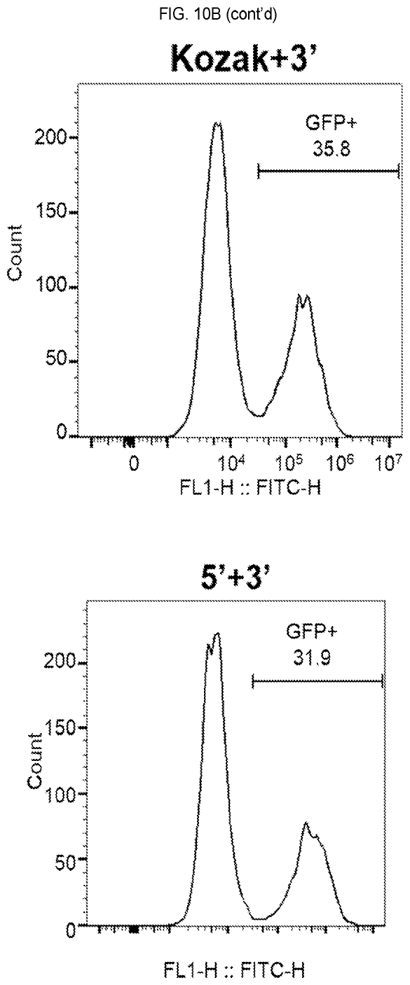

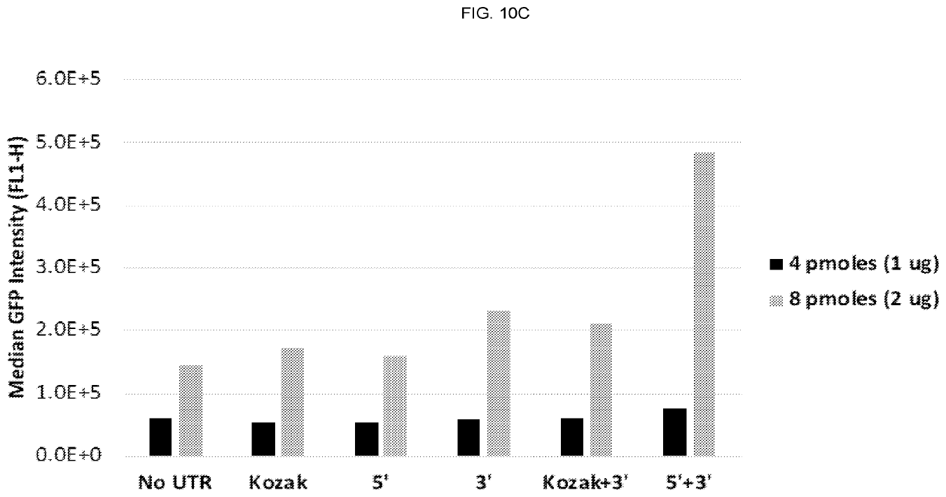

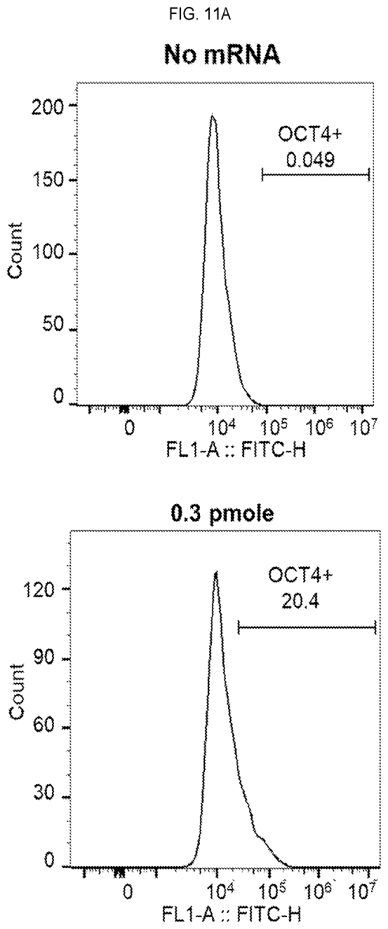

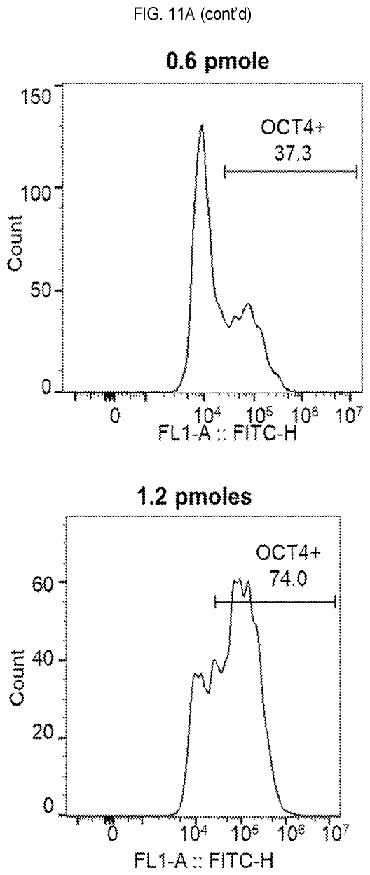

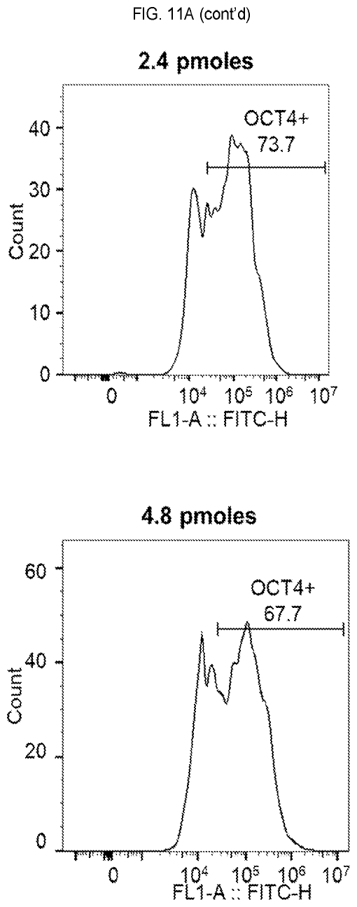

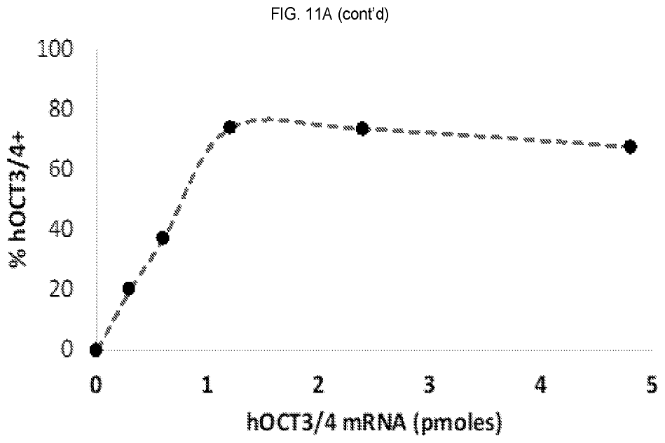

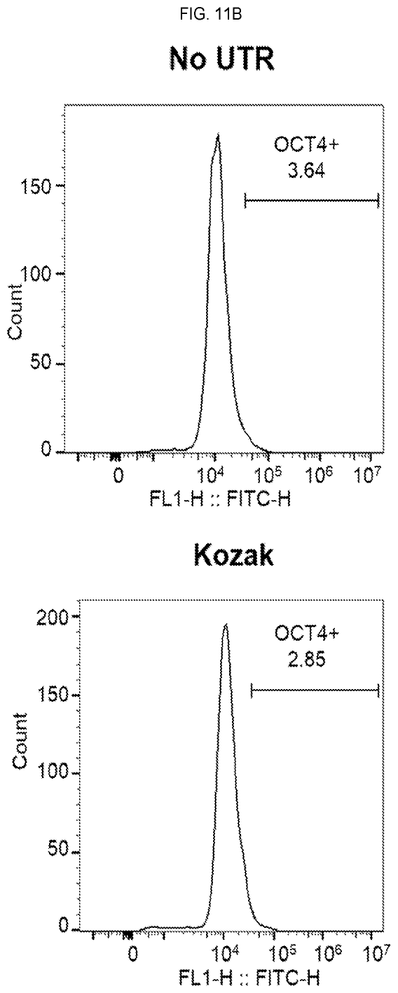

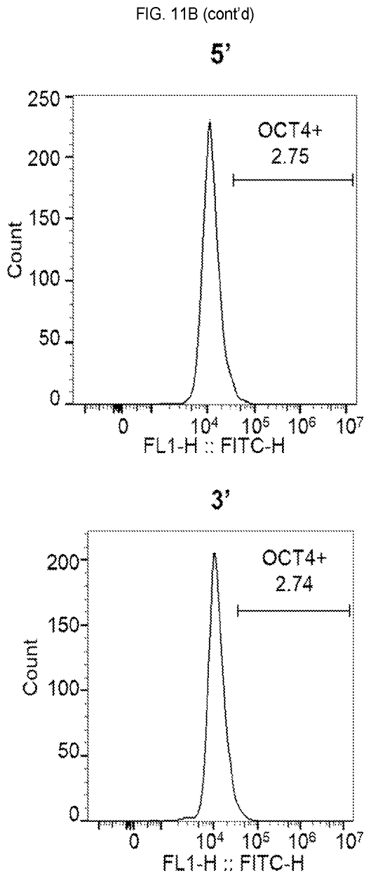

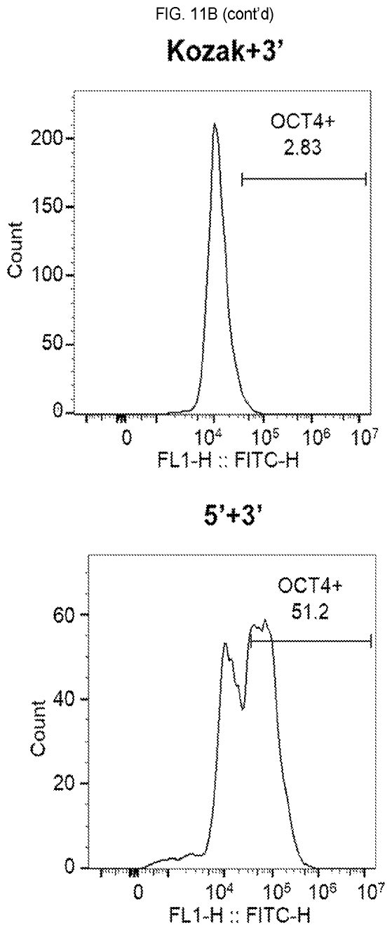

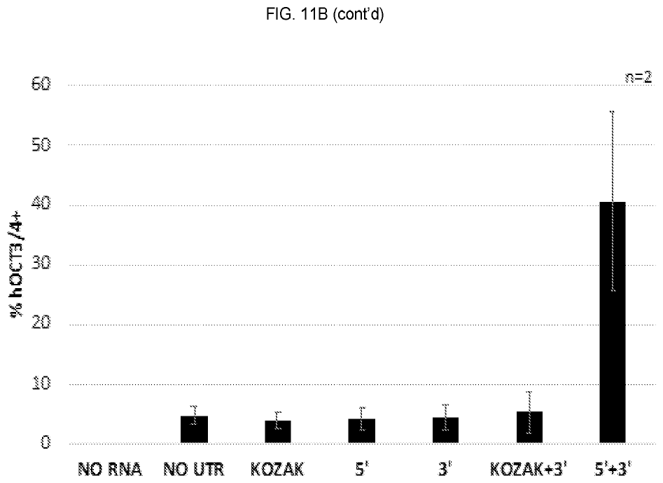

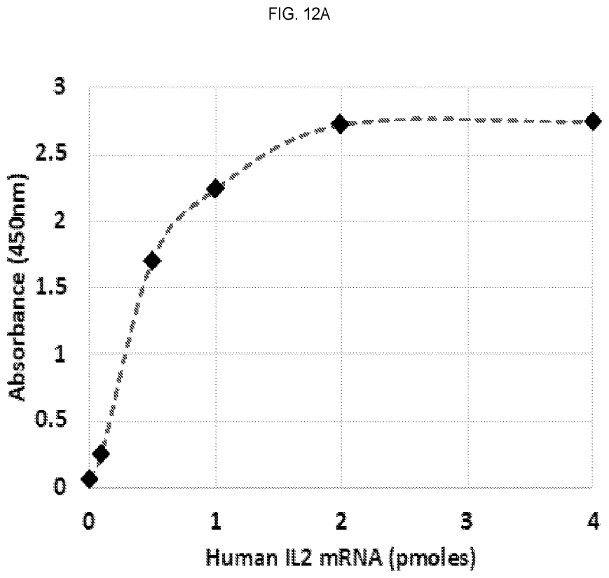

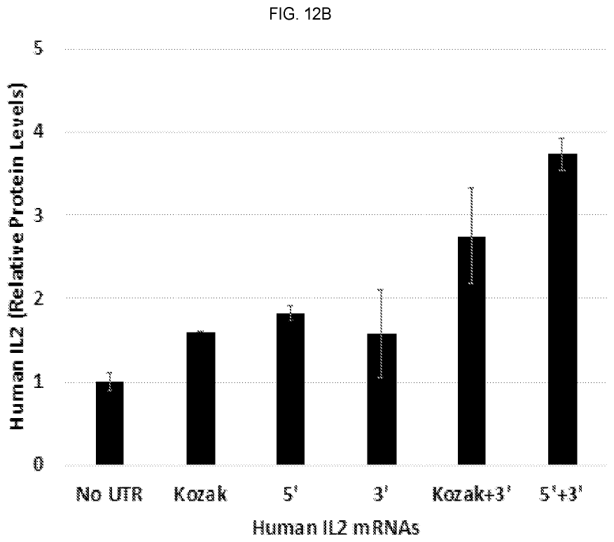

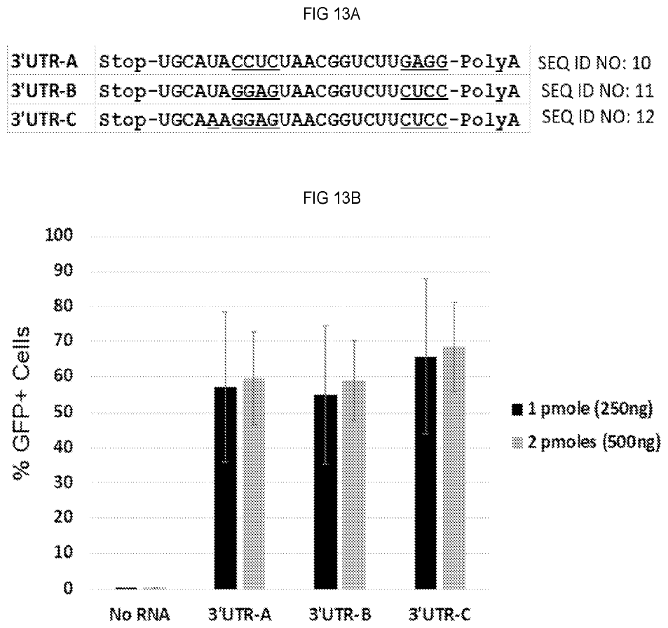

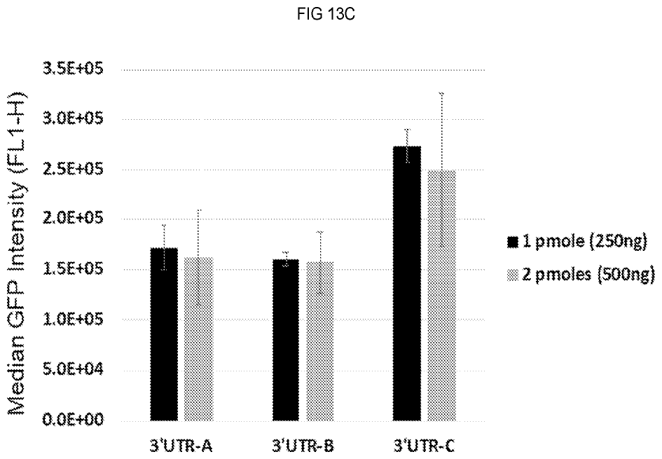

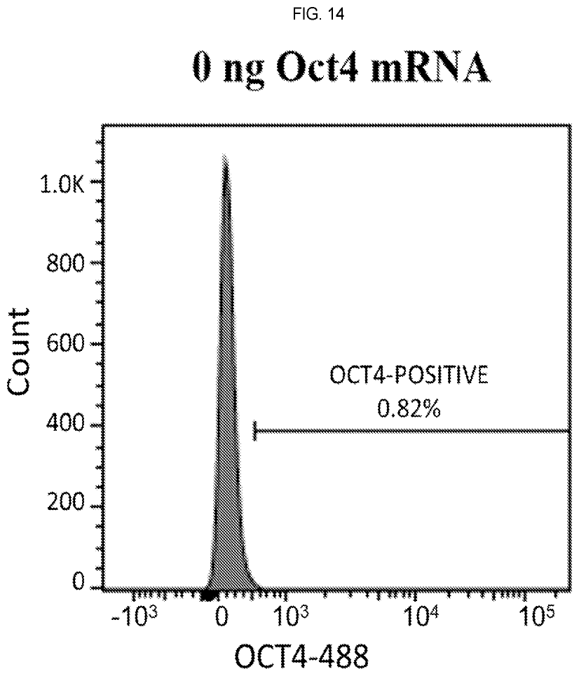

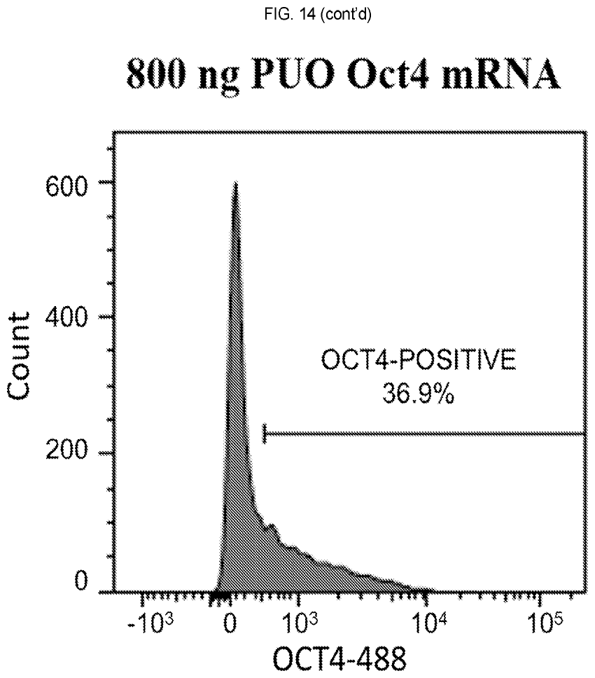

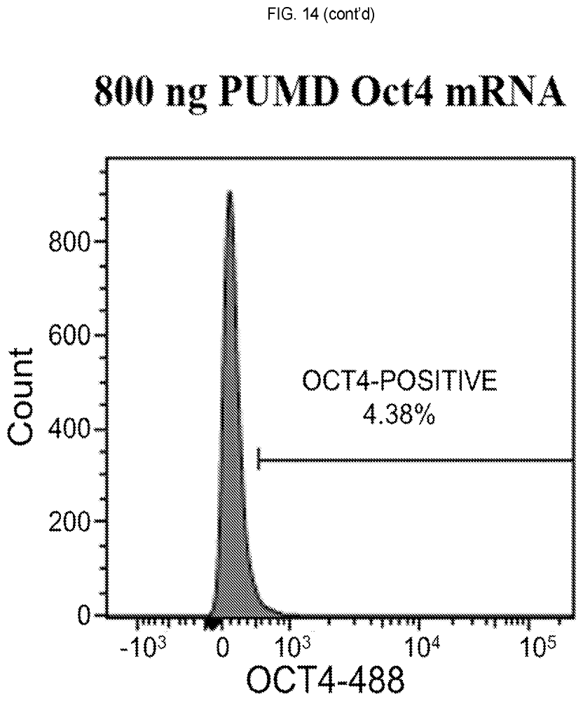

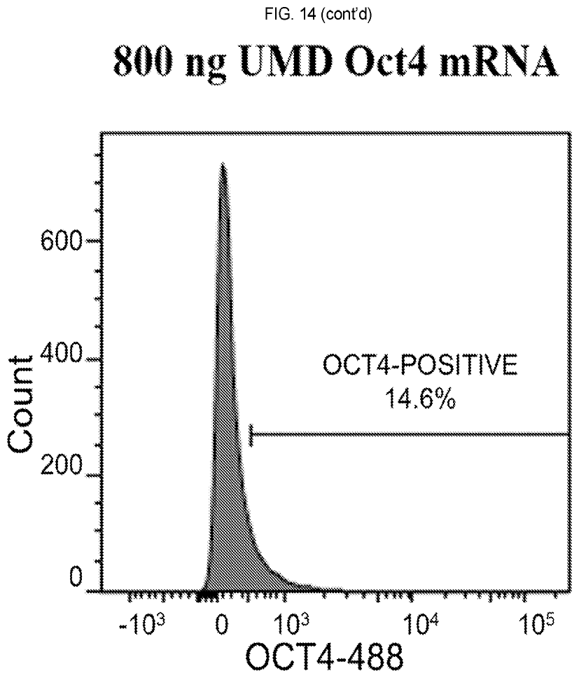

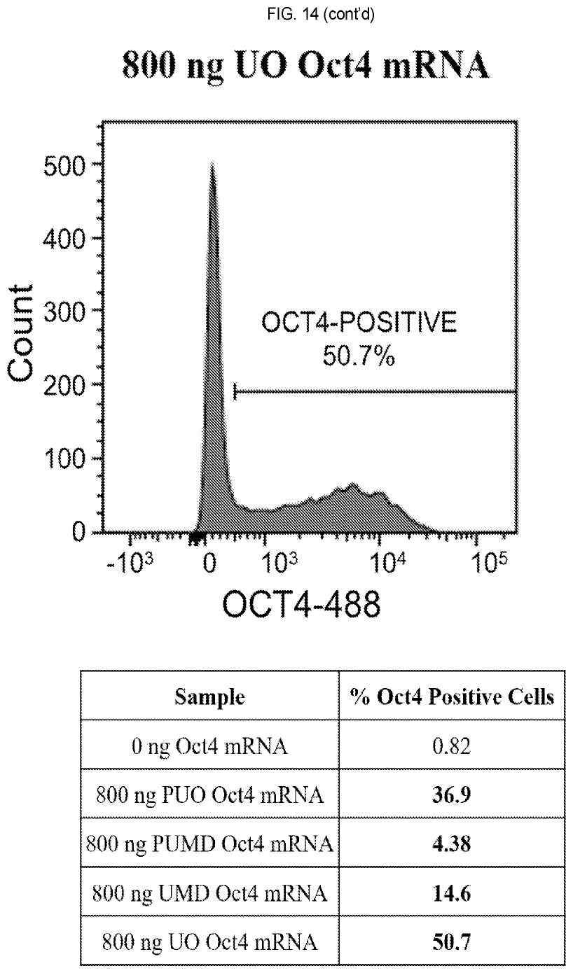

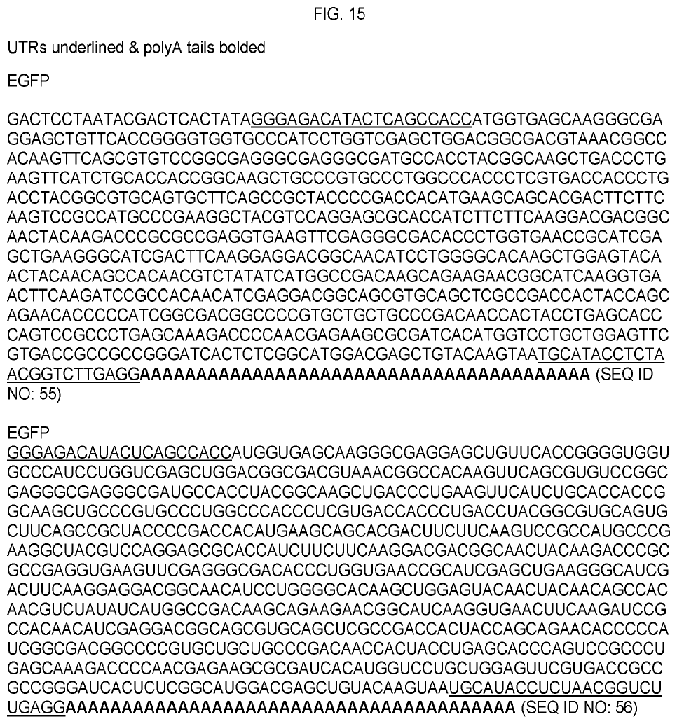

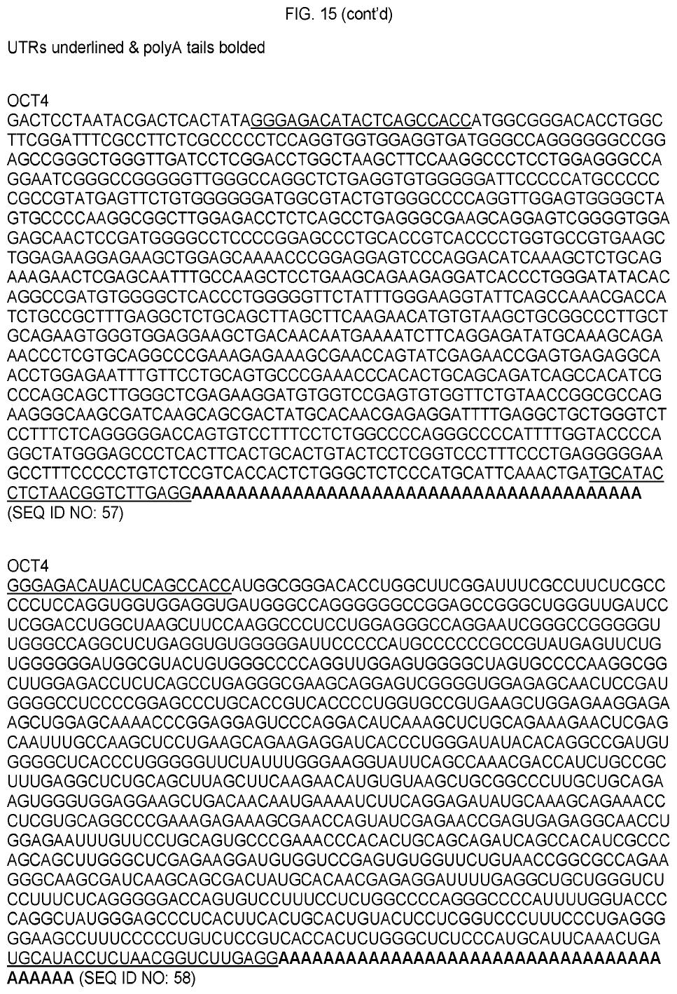

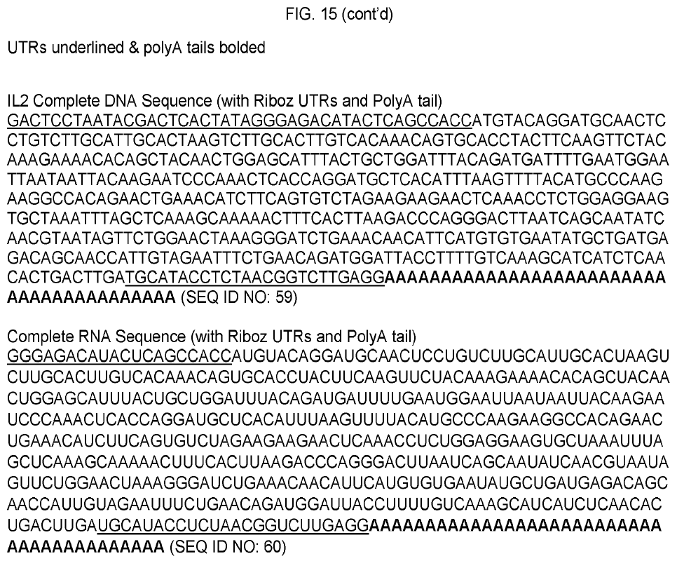

Next, cells were transfected with equimoles of mRNAs with various 5′UTR sequences, and GFP expression was analyzed at 3-hour and 24-hour time-points after transfection. The various 5′UTR sequences were either (M1) (T7 promoter)) with Kozak consensus (M3); (M1, M2 ((CAUACUCA), and M3), or without any additional 5′UTR, beyond the 5′ methyl cap and the T7 hexamer. Importantly, every construct contains the 5′ methyl cap and T7 hexamer. FIGS. 3A and 3B show the results from this experiment. Here, 3A and 3B show flow cytometry graphs displaying GFP intensity (FL1-H) on the x-axis and cell counts on the y-axis for GFP-encoding EEC 5′UTR variants at three-hour post transfection (3A) and at 24 hours post transfection (3B) as well as bar graphs depicting the data. As expected, cells transfected with transcripts containing no UTRs (those constructs that only contain the T7 hexamer GGGAGA) displayed lowest median intensity of GFP signal at all time-points after transfection (FIGS. 3A, 3B). The addition of the Kozak consensus sequence with A in the R position increased protein expression by 200%, whereas further addition of the unique translational enhancer (CAUACUCA) increased GFP expression to 635% and 240% at the 3-hour and 24-hour time-points, respectively, above the GFP signal detected in cells treated with no 5′UTR transcripts.

Thus, these experiments demonstrate that the unique, engineered 5′ UTRs, especially those harboring the unique translation enhancer, were crucial to dramatically increase protein expression.

Example 3. Including the unique 3′UTR sequence in the EEC with various 5′UTR sequences resulted in increased protein expression. Next, the effect of adding unique 3′UTR on GFP expression was examined. Results of this experiment are shown in FIGS. 4A and 4B. FIGS. 4A and 4B show flow cytometry graphs displaying GFP intensity (FL1-H) on the x-axis and cell counts on the y-axis for GFP-encoding EEC containing 5′UTR and 3′UTR variants at three-hour post transfection (4A) and at 24 hours post transfection (4B) as well as bar graphs depicting the data. EXPI293 cells were transfected with equimolar amount of GFP-encoding EEC 5′UTR and 3′UTR variants, no RNA (negative control) 3′UTR only mRNA (no 5′UTR), M3 only 5′UTR plus 3′UTR, and M1, M2 and M3 5′UTR plus 3′UTR. As can be seen in FIGS. 4A and 4B, cells transfected with transcripts containing only the 3′UTR displayed low levels of GFP expression at all time-points examined. The addition of Kozak consensus along with the 3′UTR results in modest increase in GFP median intensity. However, addition of full-length 5′ (SEQ ID NO: 2) UTR along with 3′ UTR (SEQ ID NO: 10) increased GFP expression by 660% and 925% at 3-hour and 24-hour time-points, respectively. When compared with cells treated with transcripts containing the full-length 5′ (SEQ ID NO: 2) UTR only, the addition of 3′ (SEQ ID NO: 10) UTR further increased GFP signal by 137%. Importantly, cells receiving mRNAs with full-length 5′ (SEQ ID NO: 2) +3′ (SEQ ID NO: 10) UTRs exhibited highest GFP intensities.

Thus, this experiment demonstrates the importance of including the inventive 3′ stem loop to the mRNA to further increase the level of protein expression, when desired.

Example 4. EEC with the unique 5′UTR and 3′UTR sequences resulted in increased protein expression using a variety of protein types. To ensure that the engineered UTRs were useful in increasing the expression of a variety of proteins, their superiority was demonstrated in coding for proteins with distinct properties. FIG. 5 illustrates the three different types of proteins that were tested in EEC disclosed herein: targeted expression of proteins in the cytoplasm (GFP), organelle (i.e. nuclear compartment; here, human POU5F1 or OCT3/4) and extracellular compartment (i.e. secretory proteins; here, IL2). To examine how the unique 5′UTR and 3′UTR sequences affected the expression of a cytoplasmic protein expression (GFP), see Examples 2 and 3; FIGS. 3A-4B.

Human POU5F1 or OCT3/4 (herein hOCT4), a key nuclear transcription factor in stem cell reprogramming (Yu et al., Induced pluripotent stem cell lines derived from human somatic cells. Science (80). (2007), doi:10.1126/science.1151526), was utilized to determine the effects of the inventive UTRs on expression of organelle-bound proteins. Similar to GFP, treatment of HEK293 cells with increasing quantity (0-4.8 pmoles) of hOCT4-encoding mRNA resulted in elevated levels of its protein within cells after 24 hours (FIGS. 11A and 11C). The percentage of hOCT4+ cells reached the maximum (75%) at 1.2 pmoles of mRNA per 2.0×10⁵ cells. By transfecting HEK293 cells with equimolar hOCT4 transcripts with 5′ and 3′ variants, only those with full-length 5′ (SEQ ID NO: 2) +3′ (SEQ ID NO: 10) UTRs displayed significant percentage of hOCT4+ cells (40%). Further, the median {number of experiments=2} hOCT4 intensity was 4-fold higher in those cells treated with hOCT4 mRNA with full-length 5′ (SEQ ID NO: 2) +3′ (SEQ ID NO: 10) UTRs (FIGS. 11A, 11B).

Finally, the effects of the inventive UTRs on the expression of a secretory protein, human Interleukin-2 (hIL2), were examined. hIL2 activates T lymphocytes and is currently a therapeutic target in autoimmune disorders and cancer (Spolski, Li, & Leonard, Biology and regulation of IL-2: from molecular mechanisms to human therapy. Nat. Rev. Immunol. (2018), doi:10.1038/s41577-018-0046-y). Similar to the above experiments, HEK293 cells were transfected with increasing levels of hIL2-encoding mRNA (including the full-length 5′ (SEQ ID NO: 2) and 3′ (SEQ ID NO: 10) UTRs). A proportional increase in hIL2 protein levels was observed by ELISA 24 hours after transfection (FIG. 11A). Further, transfection of hIL2 mRNAs with UTR variants resulted in expression of hIL2 protein with the highest levels observed when full-length 5′ (SEQ ID NO: 2) and 3′ (SEQ ID NO: 10) UTRs were both present (FIG. 11B).

Thus this experiment showed that mRNAs harboring both of the inventive, engineered UTRs was beneficial for increasing protein expression using a variety of protein types.

Example 5. EEC with the inventive 5′UTR and 3′UTR sequences resulted in increased protein expression in a variety of cell types.

To explore whether the increase in protein expression can be replicated in other cell types, the above experiments were repeated in the following cell lines: HEK293 (ATCC® CRL1573™), Jurkat (Clone E6-1; ATCC® TIB-152) and Raji (ATCCR CCL-86) lymphocytes. Adherent HEK293 cells are derived from human embryonic kidney transformed with sheared fragments of adenovirus type 5 DNA (Graham et al., Characteristics of a human cell line transformed by DNA from human adenovirus type 5. J. Gen. Virol. (1977), doi:10.1099/0022-1317-36-1-59). Seeded HEK293 cells were treated with increasing amount of a new lot of GFP-encoding EEC containing both 5′ (SEQ ID NO: 2) and 3′ (SEQ ID NO: 10) UTRs. These experiments were performed as mRNA transfections using MessengerMax Lipofectamine reagent (ThermoFisher), as described in Example 1. As shown in FIGS. 6A, 6B, and 6C, close to 90% of cells displayed GFP signal with a signal saturation at 1 pmole (250 ng) of GFP-encoding EEC. Treatment of cells with higher amounts of mRNA only slightly increased the percentage of GFP positive cells. In terms of median GFP intensity, representing the amount of protein expression, the saturation was reached at 2 pmoles (500 ng).

Subsequently, HEK293 cells were treated with 0.4-1 pmole of various EEC containing the disclosed 5′ (SEQ ID NO: 2) and 3′ (SEQ ID NO: 10) UTRs (FIGS. 7A and B). While all mRNA variants induced the expression of GFP in 60-80% of cells, only cells treated with full-length UTRs displayed 5-fold the GFP median intensity as compared to others (FIG. 7C). Median intensity was calculated from two experiments.

Next, the above experiment was repeated in lymphocyte lines, Jurkat and Raji. Jurkat are T lymphocytes were established from peripheral blood of a 14 year-old boy with acute T cell leukemia (Schneider, Schwenk, & Bornkamm, Characterization of EBV-genome negative “null” and “T” cell lines derived from children with acute lymphoblastic leukemia and leukemic transformed non-Hodgkin lymphoma. Int. J. Cancer (1977), doi:10.1002/ijc.2910190505). Raji cells are B lymphocytes from a 11-year-old male patient with Burkitt's lymphoma (Osunkoya, The preservation of burkitt tumour cells at moderately low temperature. Br. J. Cancer (1965), doi:10.1038/bjc.1965.87; Pulvertaft, A Study of Malignant Tumours In Nigeria by Short-Term Tissue Culture. J. Clin. Pathol. (1965), doi:10.1136/jcp.18.3.261). In these experiments, mRNA transfection was performed with jetMessenger reagent (Polyplus-transfection®). As opposed to HEK293 cells with 90% transfection efficiency (i.e. GFP positive cells), only 10% of Jurkat cells displayed GFP signal at highest mRNA amount (16 pmoles) (FIG. 7A). Further, whereas cells treated with equimolar of various constructs only modestly displayed GFP signal, those transfected with transcripts containing full-length 5′ (SEQ ID NO: 2) and 3′ (SEQ ID NO: 10) UTRs displayed the highest number of GFP positive cells (14%; FIG. 7B). Similar results are observed with Raji cells (data not shown).

Thus this experiment shows that the inventive, engineered UTRs are helpful in increasing protein expression in a variety of cell types.

Example 6. Method of transfection was immaterial to the protein-expression increasing effects of EEC disclosed herein. To examine whether the method of transfection was important to the expression-increasing effects seen with EEC disclosed herein, Jurkat cells were electroporated with increasing amount of EEC with coding sequences encoding GFP.

Electroporation has been shown to improve the delivery of nucleic acids into lymphoid cell lines (Ohtani et al., Electroporation: Application to human lympboid cell lines for stable introduction of a transactivator gene of human T-cell leukemia virus type I. Nucleic Acids Res. (1989), doi:10.1093/nar/17.4.1589). First, to determine the optimal amount of mRNA used in the experiment, electroporation was conducted with the Neon Electroporation System (Thermo Fisher Scientific, Inc.) as described in Example 1, for Jurkat cells with increasing amount of GFP-encoding EEC. This resulted in proportional increase in the GFP signal. Next, Jurkat cells were subjected to electroporation with 4 and 8 pmoles of GFP-encoding EEC with UTR variants. At 4 pmoles (1 μg) of GFP-encoding EEC per 6-8×10⁵ cells, a maximum of 10% of cells were GFP positive with those cells treated with mRNA harboring full-length 5′ (SEQ ID NO: 2) and 3′ (SEQ ID NO: 10) UTRs demonstrating the highest percentage of GFP+ cells (FIG. 9A). At 8 pmoles (2 μg), close to 90% of Jurkat cells are GFP positive with those cells treated with mRNA harboring full-length 5′ (SEQ ID NO: 2) and 3′ (SEQ ID NO: 10) UTRs displaying highest median GFP intensity (FIGS. 9B and 9C). Similar results were obtained when using Raji cells, except the number of GFP positive cells were lower at highest mRNA levels used (FIGS. 10A, 10B, and 10C).

Because similar levels of protein expression were observed in Example 7 using the unique, engineered EEC as compared to the results in Examples 2, 3, and 4, the methods of transfection do not impact the effectiveness of the EEC.

Example 7. Reversing the sequence of the stem loop on the 3′UTR had no effect on the increase in protein expression. To examine the impact of 3′UTR sequence composition, while keeping the stem-loop pairing conserved, on protein expression, the original 3′UTR sequence (FIG. 13A, 3′UTR-A) was edited to exchange CCUC with GAGG; FIG. 13A, 3′UTR-B). GFP-encoding EEC were constructed to include either 3′UTR-A or 3′UTR-B and tested for GFP expression in HEK293 cells (by transfection with MessengerMax lipofectamine). Using 1-2 pmoles, 60-70% of cells exhibited GFP expression {number of experiments=2-3} with no difference between constructs harboring either 3′UTRs (FIG. 13B). Moreover, the GFP median intensity {number of experiments=2-3} was also similar between EEC with either 3′UTR-A or 3′UTR-B (FIG. 13C). GFP expression was also examined using an EEC with an additional 3′UTR where a single nucleotide substitution (U-to-A) occurs at −2 position before the GGAG in 3′UTR-B (FIG. 13A, 3′UTR-C). This sequence resembles the histone stem-loop where the stem region is preceded by a string of adenosines important for mRNA association with stem-loop binding protein (SLBP) and translation (Battle & Doudna, The stem-loop binding protein forms a highly stable and specific complex with the 3′ stem-loop of histone mRNAs. RNA (2001), doi:10.1017/S1355838201001820; William & Marzluff, The sequence of the stem and flanking sequences at the 3′ end of histone mRNA are critical determinants for the binding of the stem-loop binding protein. Nucleic Acids Res. (1995), doi:10.1093/nar/23.4.654). While the addition of 3′UTR-C did not increase the percentage of GFP positive cells as compared to previous 3′UTRs, it did increase the GFP median intensity by 60% in transfected cells (FIGS. 13B, 13C).

Therefore, as observed with previous examples, engineered 3′UTRs including a stem loop with unique flanking sequence increase GFP expression in human cells.

Example 8. The engineered mRNA containing the unique 5′UTR sequences resulted in increased protein expression when compared to mRNA using modified nucleotides when transfected into fibroblasts.

To compare the level of protein produced from the disclosed engineered mRNA with that of mRNA using modified nucleotides (N1-methyl-pseudouridine), several Oct4 expressing mRNAs constructs were transfected into human foreskin fibroblasts, including unmodified mRNA Oct4 (UO), unmodified mRNA MyoD-Oct4 (UMD), modified mRNA Oct4 (PUO), and modified mRNA MyoD-Oct4 (PUMD). As shown in FIG. 14, OCT4 expression was the greatest using the unmodified mRNA (UO). The unmodified, engineered transcripts using 800 ng of mRNA resulted in the highest percentage of OCT4-positive cells at 50.7%. Further, as shown in FIG. 14, the percentage of OCT4-positive cells was significantly lower using the modified nucleoside pseudourdine (PUO and PUMD) than the currently-disclosed engineered mRNAs (36.9% compared to 50.7%). These results show that the disclosed 5′ and 3′UTRs result in higher levels of protein expression than transcripts made with modified nucleotides, like N1-methyl-pseudouridine.

See FIG. 15 for sequences used within the experimental examples.

Closing Paragraphs. As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transitional phrase “consisting essentially of” limits the scope of the embodiment to the specified elements, steps, ingredients or components and to those that do not materially affect the embodiment. As used herein, a material effect would cause a statistically significant reduction in increased protein expression observed with EEC containing SEQ ID NO: 2 in the 5′ UTR and SEQ ID NO: 10 in the 3′ UTR.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Variants of the proteins and EEC (including 5′ and 3′ UTR) disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to a reference sequence.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An engineered expression construct (EEC) having a coding sequence operably linked to a 5′ untranslated region (UTR) consisting of the sequence as set forth in SEQ ID NO:38 and a 3′ UTR consisting of the sequence as set forth in SEQ ID NO: 13, 14, or 15.

2. An engineered expression construct (EEC) having a 5′ untranslated region (UTR) operably linked to a coding sequence, wherein the 5′ UTR has the sequence as set forth in CAUACUCA in between a minimal promoter and a Kozak sequence.

3. The EEC of claim 2, wherein the minimal promoter is a T7 promoter.

4. The EEC of claim 3, wherein the T7 promoter has the sequence as set forth in GGGAGA.

5. The EEC of claim 2, wherein the Kozak sequence has the sequence as set forth in GCCRCCAUG, wherein R is A or G.

6. The EEC of claim 2, wherein the 5′ UTR has
(i) the sequence as set forth in SEQ ID NO: 2 operably linked to a start codon or
(ii) the sequence as set forth in SEQ ID NO: 3 operably linked to a start codon.

7. The EEC of claim 6, wherein the sequence as set forth in SEQ ID NO: 2 operably linked to a start codon has the sequence as set forth in SEQ ID NO: 38.

8. The EEC of claim 6, wherein the sequence as set forth in SEQ ID NO: 3 operably linked to a start codon has the sequence as set forth in SEQ ID NO: 39.

9. The EEC of claim 2, wherein the 5′ UTR is less than 30 nucleotides.

10. The EEC of claim 2, further comprising a 3′ UTR.

11. The EEC of claim 10, wherein the 3′ UTR comprises a spacer, and a stem loop structure operably linked to a stop codon.

12. The EEC of claim 11, wherein the stop codon has the sequence UAA, UGA, or UAG.

13. The EEC of claim 11, wherein the spacer has the sequence [N₁₋₃]AUA or [N₁₋₃]AAA.

14. The EEC of claim 11, wherein the spacer has the sequence UGCAUA or UGCAAA.

15. The EEC of claim 11, wherein the stem loop structure has hybridizing sequences as set forth in CCUC and GAGG.

16. The EEC of claim 11, wherein the stem loop structure has hybridizing sequences as set forth in AAACCUC and GAGG or as set forth in AAAGAGG and CCUC.

17. The EEC of claim 11, wherein the stem loop structure has a loop segment having at least 7 nucleotides.

18. The EEC of claim 11, wherein the stem loop structure has a loop segment having 7-15 nucleotides.

19. The EEC of claim 11, wherein the stem loop structure has a loop segment having the sequence as set forth in UAACGGUCUU (SEQ ID NO: 34).

20. The EEC of claim 10, wherein the 3′ UTR is less than 30 nucleotides.

21. The EEC of claim 10, wherein the 3′ UTR further comprises a polyadenine (polyA) tail.

22. The EEC of claim 21, wherein the polyA tail has 60 residues or less.

23. The EEC of claim 21, wherein the polyA tail has 40 residues.

24. The EEC of claim 10, wherein the 3′ UTR has the sequence as set forth in SEQ ID NO: 4, 5, 6, 7, 8, or 9.

25. The EEC of claim 10, wherein the 3′ UTR has the sequence as set forth in SEQ ID NO: 10, 11, or 12.

26. The EEC of claim 10, wherein the 3′ UTR has the sequence as set forth in SEQ ID NO: 13, 14, 15, 16, 17, 18, 19, 20, or 21.

27. The EEC of claim 2, wherein the EEC comprises in vitro-synthesized messenger RNA (mRNA).

28. The EEC of claim 2, wherein the coding sequence encodes Green Fluorescent Protein (GFP), Human Interleukin-2 (IL2) or Human POU5F1 (or OCT3/4).

29. The EEC of claim 2, having the sequence as set forth in SEQ ID NO: 56, 58, or 60.

30. The EEC of claim 2, wherein the coding sequence encodes a therapeutic protein.

31. The EEC of claim 30, wherein the therapeutic protein comprises an antibody or binding fragment thereof.

32. The EEC of claim 31, wherein the antibody or binding fragment thereof comprises an anti-SARS-Cov2 antibody or binding fragment thereof, an anti-SARS antibody or binding fragment thereof, an anti-RSV antibody or binding fragment thereof, an anti-HIV antibody or binding fragment thereof, an anti-Dengue virus antibody or binding fragment thereof, an anti-Bordatella pertussis antibody or binding fragment thereof, an anti-hepatitis C antibody or binding fragment thereof, an anti-influenza virus antibody or binding fragment thereof, an anti-parainfluenza virus antibody or binding fragment thereof, an anti-metapneumovirus (MPV) antibody or binding fragment thereof, an anti-cytomegalovirus antibody or binding fragment thereof, an anti-Epstein Barr virus antibody; anti-herpes simplex virus antibody or binding fragment thereof, an anti-Clostridium difficile bacterial toxin antibody or binding fragment thereof, or an anti-tumor necrosis factor (TNF) antibody or binding fragment thereof.

33. The EEC of claim 2, wherein the coding sequence encodes a vaccine antigen.

34. The EEC of claim 33, wherein the vaccine antigen comprises a SARS-CoV-02 vaccine antigen, a CMV vaccine antigen, an EBV vaccine antigen, a hepatitis vaccine antigen, a herpes simplex vaccine antigen, a human immunodeficiency virus (HIV), vaccine antigen, a human papillomavirus virus (HPV) viral antigen, an influenza vaccine antigen, a Japanese encephalitis vaccine antigen, a malaria vaccine antigen, a measles vaccine antigen, a rabies vaccine antigen, a respiratory syncytial vaccine antigen, a rotaviral vaccine antigen, a varicella zoster vaccine antigen, or a zika vaccine antigen.

35. The EEC of claim 2, wherein the coding sequence encodes a cytokine.

36. The EEC of claim 2, wherein the coding sequence encodes a cell-penetrating protein.

37. Thee EEC of claim 36, wherein the cell-penetrating protein comprises penetratin, the minimal domain of TAT, VP22, ZEBRA, melittin, mastoporan, maurocalcin, crotamine, buforin, poly-arginine, or transportan.

38. The EEC of claim 2, wherein the EEC does not comprise modified nucleosides.

39. The EEC of claim 2, wherein the EEC does not comprise microRNA binding sites.

40. The EEC of claim 2, wherein the coding sequence encodes an immune evading factor.

41. The EEC of claim 41, wherein the immune evading factor comprises B18R, E3, K3, NS1, or ORF8.

42. The EEC of claim 2, wherein the EEC does not comprise immune evading factors.

43. The EEC of claim 2, formulated for administration to a subject.

44. An enhancer sequence consisting of the sequence as set forth in CAUACUCA.

45. An engineered expression (EEC) construct having 1, 2, 3, 4, or 5 copies of the sequence as set forth in CAUACUCA.

46. The EEC of claim 44, wherein the enhancer sequence is operably linked to a promoter.

47. The EEC of claim 46, wherein the promoter is a minimal promoter.

48. An engineered expression construct comprising an in vitro-synthesized RNA comprising a coding sequence within an open reading frame that encodes a protein for translation in a mammalian cell, wherein said in vitro-synthesized RNA further comprises one of a 5′ untranslated region comprising CAUACUCA and a 3′ untranslated region comprising one of SEQ ID NOs: 4, 5, 6, or 7.

49. An engineered expression construct comprising an in vitro-synthesized RNA comprising a coding sequence within an open reading frame that encodes a protein for translation in a mammalian cell, wherein said in vitro-synthesized RNA further comprises one of a 5′ untranslated region comprising SEQ ID NO: 2, or SEQ ID NO: 3 and a 3′ untranslated region comprising SEQ ID NOs: 4, 5, 6, or 7.

50. An engineered expression construct comprising an in vitro-synthesized RNA comprising an open reading frame that encodes a protein for translation in a mammalian cell, wherein said in vitro-synthesized RNA further comprises a 5′ untranslated region comprising a T7 polymerase promoter, the sequence as set forth in CAUACUCA, and a Kozak sequence.

51. The engineered expression construct of claim 2, wherein the T7 promoter is selected from a T7 Class III promoter.

52. An engineered expression construct comprising an in vitro-synthesized RNA comprising an open reading frame that encodes a protein for translation in a mammalian cell, wherein said in vitro-synthesized RNA comprises a 3′ untranslated region comprising SEQ ID NOs: 4, 5, 6, or 7 and a stop codon.

53. The engineered expression construct of claim 52, wherein the stop codon is UAA, UAG, or UGA.

54. An engineered expression construct comprising an in vitro-synthesized RNA comprising an open reading frame that encodes a protein for translation in a mammalian cell, wherein said in vitro-synthesized RNA comprises a 3′ untranslated region comprising either a) CCUC and GAGG or b) GAGG and CCUC, wherein either set of the 3′ untranslated region sequences is separated by no fewer than seven nucleotides.

55. An engineered expression construct (EEC) comprising an in vitro-synthesized RNA comprising an open reading frame that encodes a protein for translation in a mammalian cell, wherein said in vitro-synthesized RNA comprises a 3′ untranslated region comprising either a) AAACCUC and GAGG or b) AAAGAGG and CCUC, wherein either set of the 3′ untranslated region sequences is separated by no fewer than seven nucleotides.

56. A 5′ untranslated region (UTR) comprising a sequence as set forth in CAUACUCA that is in between a minimal promoter and a Kozak sequence.

57. The 5′UTR of claim 56, wherein the minimal promoter is a T7 promoter.

58. The 5′UTR of claim 57, wherein the T7 promoter has the sequence as set forth in GGGAGA.

59. The 5′UTR of claim 56, wherein the Kozak sequence has the sequence as set forth in GCCRCCAUG, wherein R is adenosine or guanine.

60. The 5′UTR of claim 56, comprising the sequence as set forth in SEQ ID NO: 2 operably linked to a start codon.

61. The 5′UTR of claim 56, comprising the sequence as set forth in SEQ ID NO: 3 operably linked to a start codon.

62. The 5′UTR of claim 60, wherein the sequence as set forth in SEQ ID NO: 2 operably linked to a start codon has the sequence as set forth in SEQ ID NO: 38.

63. The 5′UTR of claim 61, wherein the sequence as set forth in SEQ ID NO: 3 operably linked to a start codon has the sequence as set forth in SEQ ID NO: 39.

64. The 5′UTR of claim 56, wherein the 5′ UTR is less than 30 nucleotides.

65. A 3′UTR comprising a spacer and a stem loop structure operably linked to a stop codon, wherein the stop codon has the sequence UAA, UGA, or UAG and the spacer has the sequence [N₁₋₃]AUA or [N₁₋₃]AAA.

66. The 3′UTR of claim 65, wherein the spacer has the sequence UGCAUA or UGCAAA.

67. The 3′UTR of claim 65, wherein the stem loop structure comprises hybridizing sequences as set forth in CCUC and GAGG.

68. The 3′UTR of claim 65, wherein the stem loop structure comprises hybridizing sequences as set forth in AAACCUC and GAGG or as set forth in AAAGAGG and CCUC.

69. The 3′UTR of claim 65, wherein the stem loop structure has a loop segment having at least 7 nucleotides.

70. The 3′UTR of claim 65, wherein the stem loop structure has a loop segment having 7-15 nucleotides.

71. The 3′UTR of claim 65, wherein the stem loop structure has a loop segment having the sequence as set forth in UAACGGUCUU (SEQ ID NO: 34).

72. The 3′UTR of claim 65, wherein the 3′ UTR is less than 30 nucleotides.

73. The 3′UTR of claim 65, wherein the 3′ UTR further comprises a polyadenine (polyA) tail.

74. The 3′UTR of claim 73, wherein the polyA tail has 60 residues or less.

75. The 3′UTR of claim 73, wherein the polyA tail has 40 residues.

76. The 3′UTR of claim 65, wherein the 3′ UTR has the sequence as set forth in SEQ ID NO: 4, 5, 6, 7, 8, or 9.

77. The 3′UTR of claim 65, wherein the 3′ UTR has the sequence as set forth in SEQ ID NO: 10, 11, or 12.

78. The 3′UTR of claim 65, wherein the 3′ UTR has the sequence as set forth in SEQ ID NO: 13, 14, 15, 16, 17, 18, 19, 20, or 21.

79. The 3′UTR of claim 65, wherein the 3′UTR is operably linked to a coding sequence.

80. The 3′UTR of claim 79, wherein the coding sequence encodes a therapeutic protein, a vaccine antigen, a cytokine, or a fluorescent protein.

81. The 3′UTR of claim 79, wherein the coding sequence encodes an immune evading factor.

82. The 3′UTR of claim 81, wherein the immune evading factor comprises B18R, E3, K3, NS1, or ORF8.

83. The 3′UTR of claim 79, wherein the coding sequence does not comprise an immune evading factor.

84. The 3′UTR of claim 79, wherein the coding sequence encodes a cell-penetrating protein.

85. The 3′UTR of claim 84, wherein the cell-penetrating protein comprises penetratin, the minimal domain of TAT, VP22, ZEBRA, melittin, mastoporan, maurocalcin, crotamine, buforin, poly-arginine, or transportan.
